(19) United States
(12) Patent Application Publication
(10) Pub. No.: US 2026/0102490 A1
(43) Pub. Date: Apr. 16, 2026

(54) COMBINATION OF TRISPECIFIC ANTIBODY TARGETING BCMA, GPRC5D AND CD3, AND AN ANTI-CD38 ANTIBODY FOR THE TREATMENT OF MULTIPLE MYELOMA

(71) Applicant: Janssen Biotech, Inc., Horsham, PA (US)

(72) Inventor: Sangmin Lee, Cumming, GA (US)

(21) Appl. No.: 19/355,707

(22) Filed: Oct. 10, 2025

Related U.S. Application Data
(60) Provisional application No. 63/706,347, filed on Oct. 11, 2024.
(60) Provisional application No. 63/738,277, filed on Dec. 23, 2024.
(60) Provisional application No. 63/809,555, filed on May 21, 2025.

(51) Int. Cl. A61K 39/395; A61K 39/00; A61P 35/00; C07K 16/28

(52) U.S. Cl. CPC A61K 39/39558; A61P 35/00; C07K 16/2809; C07K 16/2866; C07K 16/2878; C07K 16/2896; A61K 2039/507; A61K 2039/545; C07K 2317/24; C07K 2317/31; C07K 2317/569; C07K 2317/92

(57) ABSTRACT

Embodiments disclosed herein relate to methods of treating multiple myeloma in a subject in need thereof, comprising administering a therapeutically effective amount of BCMA×GPRC5D×CD3 trispecific antibody or trispecific binding fragment thereof and an anti-CD38 antibody, to the subject to the subject to treat the multiple myeloma.

### CROSS-REFERENCE TO RELATED APPLICATIONS

This application claims the benefit of U.S. Provisional Application Ser. No. 63/706,347 filed on 11 Oct. 2024, 63/738,277 filed on 23 Dec. 2024, and 63/809,555 filed on 21 May 2025. The entire contents of the above-referenced applications are incorporated by reference in their entireties.

### SEQUENCE LISTING

The instant application contains a Sequence Listing which has been submitted electronically in XML file format and is hereby incorporated by reference in its entirety. Said XML copy, created on 6 Oct. 2025, is named JBI6955USNP1_SL.xml and 90,246 bytes in size.

### TECHNICAL FIELD

The disclosure provided herein relates to methods of treating multiple myeloma using a combination of trispecific antibodies or trispecific binding fragments thereof that bind B-cell maturation antigen (BCMA), G-protein coupled receptor, class C, group 5, member D (GPRC5D), and cluster determinant 3 (CD3) and an anti-CD38 antibody.

### BACKGROUND

Multiple myeloma (MM) is a plasma cell cancer, accounting for 10% of hematologic malignancies and 2% of cancer deaths. It often arises from precursor conditions, affecting 3% of adults over 50. MM is a genetically heterogeneous disease, with common chromosome translocations (e.g., t(11;14), t(4;14)) and is more prevalent in men and people of African descent. Symptoms result from bone marrow infiltration, bone destruction, renal failure, and immunodeficiency. Despite advances in treatment, MM remains incurable, with a 5-year survival rate of 34% as of 2006.

B-cell maturation antigen (BCMA), a receptor on B cells, plays a key role in plasma cell survival and is a validated therapeutic target in MM, leading to several approved therapies. BCMA interacts with ligands APRIL and BAFF to promote B cell survival and inhibit apoptosis. Soluble BCMA levels correlate with disease severity, and its inhibition has therapeutic potential.

GPRC5D, a plasma cell-expressed G protein-coupled receptor, is another emerging MM target. Its expression in malignant plasma cells, coupled with limited expression in normal tissues, makes it ideal for therapeutic interventions.

CD38, another key target in MM, is involved in immune cell adhesion, signaling, and calcium mobilization. Anti-CD38 antibodies are also approved treatments.

Current MM therapies include proteasome inhibitors, immunomodulatory drugs, monoclonal antibodies, and CAR-T therapies. However, the disease remains incurable, with relapse often due to clonal resistance. Targeting multiple antigens, such as BCMA and GPRC5D, in combination therapies may improve outcomes, addressing antigen escape and enhancing treatment efficacy. There remains an unmet need for novel therapies, particularly for elderly patients and those with refractory disease.

### SUMMARY

Provided herein is a method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is administered at a treatment dose of at least about 20 mg.

In one aspect, provided herein is a method of treating multiple myeloma in a subject in need thereof, comprising administering a therapeutically effective amount of a BCMA×GPRC5D×CD3 trispecific antibody or a trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of at least about 20 mg.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 20 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 50 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 100 mg and wherein the treatment dose is administered once every four weeks.

In some embodiments, the method further comprises administering lenalidomide to the subject. In some embodiments, lenalidomide is administered orally at a dose of about 25 mg daily for 21 days during a 28-day cycle, commencing on Cycle 2 Day 1. In some embodiments, the dose of lenalidomide is adjusted based on patient tolerance and hematologic toxicity. In some embodiments, dexamethasone is administered in conjunction with lenalidomide. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 200 mg and wherein the treatment dose is administered once every four weeks.

In some embodiments, the method further comprises administering lenalidomide to the subject. In some embodiments, lenalidomide is administered orally at a dose of about 25 mg daily for 21 days during a 28-day cycle, commencing on Cycle 2 Day 1. In some embodiments, the dose of lenalidomide is adjusted based on patient tolerance and hematologic toxicity. In some embodiments, dexamethasone is administered in conjunction with lenalidomide. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 300 mg and wherein the treatment dose is administered once every four weeks.

In some embodiments, the method further comprises administering lenalidomide to the subject. In some embodiments, lenalidomide is administered orally at a dose of about 25 mg daily for 21 days during a 28-day cycle, commencing on Cycle 2 Day 1. In some embodiments, the dose of lenalidomide is adjusted based on patient tolerance and hematologic toxicity. In some embodiments, dexamethasone is administered in conjunction with lenalidomide. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

In some embodiments, the multiple myeloma is a relapsed or refractory form of multiple myeloma. In some embodiments, the multiple myeloma is a relapsed form of multiple myeloma. In some embodiments, the multiple myeloma is a refractory form of multiple myeloma.

In an embodiment of any of the methods disclosed herein, the BCMA×GPRC5D×CD3 trispecific antibody or trispecific binding fragment thereof is the trispecific antibody formed from the amino acid chains having the sequences of SEQ ID Nos: 29, 30 and 31 (this molecule is also herein termed BGCB491; see also the structure in FIG. 14).

In one embodiment provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein daratumumab is formulated in a pharmaceutical composition comprising 1800 mg daratumumab and 30000 units hyaluronidase and is administered subcutaneously and wherein the subject is a human subject.

Provided herein is a pharmaceutical composition comprising a BCMA×GPRC5D×CD3 trispecific antibody or a trispecific binding fragment thereof for use in treating multiple myeloma, wherein the composition is for administration, or to be administered, in combination with a pharmaceutical composition comprising an anti-CD38 antibody, and further wherein the BCMA×GPRC5D×CD3 trispecific antibody or trispecific binding fragment thereof is for subcutaneous administration at a treatment dose of at least about 20 mg. In some embodiments, the pharmaceutical composition further comprises lenalidomide.

Provided herein is a pharmaceutical composition comprising an anti-CD38 antibody for use in treating multiple myeloma, wherein the composition is for administration, or to be administered, in combination with a pharmaceutical composition comprising a BCMA×GPRC5D×CD3 trispecific antibody or a trispecific binding fragment thereof, and further wherein the BCMA×GPRC5D×CD3 trispecific antibody or trispecific binding fragment thereof is for subcutaneous administration at a treatment dose of at least about 20 mg. In some embodiments, the pharmaceutical composition further comprises lenalidomide.

The skilled person will understand that the described methods may be specified in medical use format, for example in the form of BCMA×GPRC5D×CD3-trispecific antibodies and trispecific antigen-binding fragments in combination with an anti-CD38 antibody for use in the treatment of multiple myeloma. This skilled person will also understand that the methods may be specified in so-called Swiss form, for example in the form of the use of BCMA×GPRC5D×CD3-trispecific antibodies and trispecific antigen-binding fragments in combination with an anti-CD38 antibody for the manufacture of a medicament for the treatment of multiple myeloma. This applies throughout the disclosure.

### BRIEF DESCRIPTION OF THE DRAWINGS

FIG. 1A, FIG. 1B, and FIG. 1C show simultaneous treatment of BGCB491 with daratumumab results in statistically significant decreased EC50 of H929 tumor cytotoxicity. CFSE-labeled H929 target cells and PBMCs of Donor 1 were co-cultured at a ratio of 3:1 E:T in the presence of increasing concentrations of BGCB491 and daratumumab for 48 h. (FIG. 1A) Dose-response curve for cytotoxicity of H929 MM cell line assessed by flow cytometry. Each point represents the mean and error bars are the standard deviation of technical triplicates. (FIG. 1B) Table detailing the EC50 values of BGCB491 with increasing concentrations of daratumumab and p-values compared to BGCB491 monotherapy. (FIG. 1C) Synergy analysis contour plot with increasing concentrations of BGCB491 along the x-axis and increasing concentrations of daratumumab along the y-axis using Highest Single Agent (HSA) synergy model.

FIG. 2A, FIG. 2B, and FIG. 2C show simultaneous treatment of BGCB491 with daratumumab results in statistically significant decreased EC50 of H929 tumor cytotoxicity. CFSE-labeled H929 target cells and PBMCs of Donor 2 were co-cultured at a ratio of 3:1 E:T in the presence of increasing concentrations of BGCB491 and daratumumab for 48 h. (FIG. 2A) Dose-response curve for cytotoxicity of H929 MM cell line assessed by flow cytometry. Each point represents the mean and error bars are the standard deviation of technical triplicates. (FIG. 2B) Table detailing the EC50 values of BGCB491 with increasing concentrations of daratumumab and p-values compared to BGCB491 monotherapy. (FIG. 2C) Synergy analysis contour plot with increasing concentrations of BGCB491 along the x-axis and increasing concentrations of Daratumumab along the y-axis using Highest Single Agent (HSA) synergy model.

FIG. 3A, FIG. 3B, and FIG. 3C show simultaneous treatment of BGCB491 with daratumumab results in statistically significant decreased EC50 of H929 tumor cytotoxicity. CFSE-labeled H929 target cells and PBMCs of Donor 3 were co-cultured at a ratio of 3:1 E:T in the presence of increasing concentrations of BGCB491 and daratumumab for 48 h. (FIG. 3A) Dose-response curve for cytotoxicity of H929 MM cell line assessed by flow cytometry. Each point represents the mean and error bars are the standard deviation of technical triplicates. (FIG. 3B) Table detailing the EC50 values of BGCB491 with increasing concentrations of daratumumab and p-values compared to BGCB491 monotherapy. (FIG. 3C) Synergy analysis contour plot with increasing concentrations of BGCB491 along the x-axis and increasing concentrations of daratumumab along the y-axis using Highest Single Agent (HSA) synergy model.

FIG. 4A, FIG. 4B, FIG. 4C, and FIG. 4D show simultaneous treatment with daratumumab has no impact on BGCB491-mediated T cell activation in H929 MM cells. CFSE-labeled H929 target cells and PBMCs of donor 1 were co-cultured at a ratio of 3:1 E:T in the presence of increasing concentrations of BGCB491 and daratumumab for 48 hours. Flow cytometry assessment of (FIG. 4A) T cell frequency expressed as percentage of total viable PBMNC. (FIG. 4B) Frequency of CD25+ CD3+ T cells expressed as percentage of total viable T cells. (FIG. 4C) Frequency of CD69+ CD3+ T cells expressed as percentage of total viable T cells. (FIG. 4D) Median Fluorescence Intensity (MFI) of CD38 on CD3+ T cells. Each point represents the mean and error bars are the standard deviation of technical triplicates.

FIG. 5A, FIG. 5B, FIG. 5C, and FIG. 5D show simultaneous treatment with daratumumab has no impact on BGCB491-mediated T cell activation in H929 MM cells. CFSE-labeled H929 target cells and PBMCs of donor 2 were co-cultured at a ratio of 3:1 E:T in the presence of increasing concentrations of BGCB491 and daratumumab for 48 hours. Flow cytometry assessment of (FIG. 5A) T cell frequency expressed as percentage of total viable PBMNC. (FIG. 5B) Frequency of CD25+ CD3+ T cells expressed as percentage of total viable T cells. (FIG. 5C) Frequency of CD69+ CD3+ T cells expressed as percentage of total viable T cells. (FIG. 5D) Median Fluorescence Intensity (MFI) of CD38 on CD3+ T cells. Each point represents the mean and error bars are the standard deviation of technical triplicates.

FIG. 6A, FIG. 6B, FIG. 6C, and FIG. 6D show simultaneous treatment with daratumumab has no impact on BGCB491-mediated T cell activation in H929 MM cells. CFSE-labeled H929 target cells and PBMCs of donor 3 were co-cultured at a ratio of 3:1 E:T in the presence of increasing concentrations of BGCB491 and daratumumab for 48 hours. Flow cytometry assessment of (FIG. 6A) T cell frequency expressed as percentage of total viable PBMNC. (FIG. 6B) Frequency of CD25+ CD3+ T cells expressed as percentage of total viable T cells. (FIG. 6C) Frequency of CD69+ CD3+ T cells expressed as percentage of total viable T cells. (FIG. 6D) Median Fluorescence Intensity (MFI) of CD38 on CD3+ T cells. Each point represents the mean and error bars are the standard deviation of technical triplicates.

FIG. 7A, FIG. 7B, FIG. 7C, and FIG. 7D show simultaneous treatment of BGCB491 and daratumumab does not impair daratumumab-mediated NK cell activation in H929 MM cells. CFSE-labeled H929 target cells and PBMCs of donor 1 were co-cultured at a ratio of 3:1 E:T in the presence of increasing concentrations of BGCB491 and Daratumumab for 48 h (FIG. 7A) NK cell frequency (FIG. 7B) Frequency of CD25+ CD56+ NK cells. (FIG. 7C) Frequency of CD69+ CD56+ NK cells (FIG. 7D) Median Fluorescence Intensity (MFI) of CD38 on CD56+ NK cells were assessed by flow cytometry. Each point represents the mean and error bars are the standard deviation of technical triplicates.

FIG. 8A, FIG. 8B, FIG. 8C, and FIG. 8D show simultaneous treatment of BGCB491 and daratumumab does not impair daratumumab-mediated NK cell activation in H929 MM cells. CFSE-labeled H929 target cells and PBMCs of donor 2 were co-cultured at a ratio of 3:1 E:T in the presence of increasing concentrations of BGCB491 and daratumumab for 48 h (FIG. 8A) NK cell frequency (FIG. 8B) Frequency of CD25+ CD56+ NK cells. (FIG. 8C) Frequency of CD69+ CD56+ NK cells (FIG. 8D) Median Fluorescence Intensity (MFI) of CD38 on CD56+ NK cells were assessed by flow cytometry. Each point represents the mean and error bars are the standard deviation of technical triplicates.

FIG. 9A, FIG. 9B, FIG. 9C, and FIG. 9D show Simultaneous treatment of BGCB491 and daratumumab does not impair daratumumab-mediated NK cell activation in H929 MM cells. CFSE-labeled H929 target cells and PBMCs of donor 3 were co-cultured at a ratio of 3:1 E:T in the presence of increasing concentrations of BGCB491 and daratumumab for 48 h (FIG. 9A) NK cell frequency (FIG. 9B) Frequency of CD25+ CD56+ NK cells. (FIG. 9C) Frequency of CD69+ CD56+ NK cells (FIG. 9D) Median Fluorescence Intensity (MFI) of CD38 on CD56+ NK cells were assessed by flow cytometry. Each point represents the mean and error bars are the standard deviation of technical triplicates.

FIG. 10A and FIG. 10B show simultaneous treatment of BGCB491 with daratumumab results in statistically significant synergy of H929 tumor cytotoxicity. (FIG. 10A AND FIG. 10B) Synergy analysis contour plots with increasing concentrations of BGCB491 along the x-axis and increasing concentrations of daratumumab along the y-axis using (FIG. 10A) Bliss Independence model and (FIG. 10B) Alternative Loewe Model. Areas of synergy are indicated.

FIG. 10C and FIG. 10D show simultaneous treatment of BGCB491 with daratumumab results in statistically significant synergy of H929 tumor cytotoxicity. (FIG. 10C AND FIG. 10D) Synergy analysis contour plots with increasing concentrations of BGCB491 along the x-axis and increasing concentrations of daratumumab along the y-axis using (FIG. 10C) Bliss Independence model and (FIG. 10D) Alternative Loewe Model. Areas of synergy are indicated.

FIG. 10E and FIG. 10F show simultaneous treatment of BGCB491 with daratumumab results in statistically significant synergy of H929 tumor cytotoxicity. (FIG. 10E AND FIG. 10F) Synergy analysis contour plots with increasing concentrations of BGCB491 along the x-axis and increasing concentrations of Daratumumab along the y-axis using (FIG. 10E) Bliss Independence model and (FIG. 10F) Alternative Loewe Model. Areas of synergy are indicated.

FIG. 11 is a depiction of the BGCB491 trispecific antibody.

FIG. 12 shows the effect of BGCB491+lenalidomide combination treatment on growth of SC RPMI-8226 human MM xenografts in T-cell humanized mice. Tumor cells were implanted on Day 0 and T cells were implanted on Day 21, followed by treatment with BGCB491 at 0.1 mg/kg (left panel), 0.4 mg/kg (right panel) or DPBS on Days 22, 25, 29, 32, 36, 39, 43, and 46 (represented by black line underneath X-axis), and treatment with lenalidomide on Days 22 to 49 (represented by gray line underneath X-axis). * Denotes significant difference of BGCB491+lenalidomide combination treated and monotherapy lenalidomide treated groups on Day 49 versus the DPBS control group. Data are displayed while at least ⅔ of animals remained in a group. Abbreviations: DPBS, Dulbecco's phosphate-buffered saline; Len, lenalidomide; MM, multiple myeloma; RPMI, Roswell Park Memorial Institute; SEM, standard error of the mean. Group tumor volumes are graphed as mean±SEM (n=10/group).

FIG. 13 shows the Effect of BGCB491, Lenalidomide or Combination Treatments on Individual RPMI 8226 Tumor Volumes in T-cell-humanized Mice. Tumor cells were implanted on Day 0 and T cells were implanted on Day 21, followed by treatment with BGCB491 or DPBS on Days 22, 25, 29, 32, 36, 39, 43, and 46, and treatment with lenalidomide on Days 22 to 49. Abbreviations: DPBS, Dulbecco's phosphate-buffered saline; RPMI, Roswell Park Memorial Institute.

### DETAILED DESCRIPTION OF ILLUSTRATIVE EMBODIMENTS

The disclosed methods can be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

In an attempt to help the reader of the present application, the description has been separated in various paragraphs or sections. These separations are not considered as disconnecting the substance of a paragraph or section from the substance of another paragraph or section. To the contrary, the present description encompasses all the combinations of the various sections, paragraphs and sentences that can be contemplated.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The term “comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to BCMA is substantially free of antibodies that specifically bind antigens other than BCMA). An isolated antibody that specifically binds to an epitope, isoform or variant of BCMA or GPRC5D may, however, have cross-reactivity to other related antigens, for instance from other species (such as BCMA or GPRC5D species homologs).

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

The meaning of “substantially the same” can differ depending on the context in which the term is used. Because of the natural sequence variation likely to exist among heavy and light chains and the genes encoding them, one would expect to find some level of variation within the amino acid sequences or the genes encoding the antibodies or antigen-binding fragments described herein, with little or no impact on their unique binding properties (e.g., specificity and affinity). Such an expectation is due in part to the degeneracy of the genetic code, as well as to the evolutionary success of conservative amino acid sequence variations, which do not appreciably alter the nature of the encoded protein. Accordingly, in the context of nucleic acid sequences, “substantially the same” means at least 65% identity between two or more sequences. Preferably, the term refers to at least 70% identity between two or more sequences, more preferably at least 75% identity, more preferably at least 80% identity, more preferably at least 85% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and more preferably at least 99% or greater identity. The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

The degree of variation that may occur within the amino acid sequence of a protein without having a substantial effect on protein function is much lower than that of a nucleic acid sequence, since the same degeneracy principles do not apply to amino acid sequences. Accordingly, in the context of an antibody or antigen-binding fragment, “substantially the same” means antibodies or antigen-binding fragments having 90%, 91%, 92%, 93%, 94%, 95%, 96%, 97%, 98%, or 99% identity to the antibodies or antigen-binding fragments described. Other embodiments include antibodies, or antigen-binding fragments, that have framework, scaffold, or other non-binding regions that do not share significant identity with the antibodies and antigen-binding fragments described herein, but do incorporate one or more CDRs or other sequences needed to confer binding that are 90%, 91%, 92%, 93%, 94%, 95%, 96%, 97%, 98%, or 99% identical to such sequences described herein.

A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression or production of an antibody or antigen-binding fragment thereof may be within the cytoplasm of the cell, or into the extracellular milieu such as the growth medium of a cell culture.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

An “effective amount” or “therapeutically effective amount” refers to an amount effective, at dosages and for periods of time necessary, to achieve a desired therapeutic result. A therapeutically effective amount of a BCMA×GPRC5D×CD3 antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

“Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

The term “antigen-binding arm” refers to a portion of an antibody that includes an antigen-binding domain that binds to an antigen (e.g., BCMA, GPRC5D, or CD3), and optionally includes one or more other antibody regions (e.g., Fc domain).

The term “antigen-binding fragment” refers to a fragment of the antigen-binding arm containing an antigen-binding domain. Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21 (11): 484-90); camelid or nanobodies (Revets et al; Expert Opin Biol Ther. 2005 January; 5 (1): 111-24); an isolated complementarity determining region (CDR), and the like; and trispecific antibodies formed from antibody fragments. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase “an antibody or antigen-binding fragment thereof” may be used to denote that a given antigen-binding fragment incorporates one or more amino acid segments of the antibody referred to in the phrase.

The term “antigen-binding domain” refers to the proteinaceous structure of an antigen-binding arm that exhibits binding affinity for a particular antigen. This proteinaceous structure is mediated by the complementarity determining regions (CDRs) of the antigen-binding domain.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable region (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to herein by Kabat, Chothia, AbM, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat et al., Sequences of Proteins of Immunological Interest, 5th ed. NIH Publication No. 91-3242 (1991); Chothia et al., “Canonical Structures For the Hypervariable Regions of Immunoglobulins,” J. Mol. Biol. 196:901 (1987); and MacCallum et al., “Antibody-Antigen Interactions: Contact Analysis and Binding Site Topography,” J. Mol. Biol. 262:732 (1996)), each of which is hereby incorporated by reference in its entirety.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three-dimensional structures, despite high amino acid sequence variability in most parts of the loops (Chothia et al., “Canonical Structures For the Hypervariable Regions of Immunoglobulins,” J. Mol. Biol. 196:901 (1987); Chothia et al., “Conformations of Immunoglobulin Hypervariable Regions,” I 342:877 (1989); Martin and Thornton, “Structural Families in Loops of Homologous Proteins: Automatic Classification, Modelling and Application to Antibodies,” J. Mol. Biol. 263:800 (1996), each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. As used herein the term “Fc” refers to the fragment crystallizable domain of an antibody, which comprises two constant heavy chain (CH) regions, CH2 and CH3. Herein, the amino acid residues of the Fc region are typically numbered according to the EU numbering scheme (Edelman, G. M. et al., Proc. Natl. Acad. USA, 63, 78-85 (1969). PMID: 5257969). These residues can be readily assigned according to alternative numbering schemes such as IMGT and Kabat (Kabat, E. A. et al., Sequences of proteins of immunological interest. 5th Edition—US Department of Health and Human Services, NIH publication n° 91-3242, pp 662680,689 (1991)) numbering as would be readily appreciated by one skilled in the art. For example, L234 according to EU numbering may also be represented as L247 according to Kabat.

“Specifically binds” or “binds specifically” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell-binding assay. Phrases such as “[antigen]-specific” antibody (e.g., BCMA-specific antibody) are meant to convey that the recited antibody specifically binds the recited antigen. Wherever the term “binds” is used herein it is intended that this encompasses “specifically binds” and the terms may be interchanged as desired.

As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

A “vector” is a replicon, such as plasmid, phage, cosmid, or virus in which another nucleic acid segment may be operably inserted so as to bring about the replication or expression of the segment.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome. The terms “expression” and “production” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In one embodiment of the described methods, the subject is a human.

The term “redirect” or “redirecting” as used herein refers to the ability of the BCMA×GPRC5D×CD3 antibody to traffic the activity of T cells effectively, from its inherent cognate specificity toward reactivity against GPRC5D and/or BCMA-expressing cells.

The term “sample” as used herein refers to a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like. The term “sample,” as used herein, encompasses materials removed from a subject or materials present in a subject. The relevant aspects of the disclosure may be performed in vitro based on isolated samples as required.

A “known standard” may be a solution having a known amount or concentration of GPRC5D and/or BCMA, where the solution may be a naturally occurring solution, such as a sample from a patient known to have early, moderate, late, progressive, or static cancer, or the solution may be a synthetic solution such as buffered water having a known amount of GPRC5D and/or BCMA diluted therein. The known standards, described herein may include GPRC5D and/or BCMA isolated from a subject, recombinant or purified GPRC5D and/or BCMA protein, or a value of GPRC5D and/or BCMA concentration associated with a disease condition.

The terms “B-cell maturation antigen” and “BCMA” as used herein include human B cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51). The term “antibody against BCMA, anti-BCMA antibody” as used herein relates to an antibody specifically binding to BCMA.

The terms “G-protein coupled receptor family C group 5 member D” and “GPRC5D” specifically include the human GPRC5D protein, for example as described in GenBank Accession No. BC069341, NCBI Reference Sequence: NP_061124.1 and UniProtKB/Swiss-Prot Accession No. Q9NZD1 (see also Brauner-Osborne, H. et al. 2001, Biochim. Biophys. Acta 1518, 237-248).

The terms “cluster determinant 3” and “CD3” include the human CD3 protein multi-subunit complex. The CD3 protein multi-subunit complex is composed to 6 distinctive polypeptide chains. These include a CD3γ chain (SwissProt P09693), a CD3δ chain (SwissProt P04234), two CD38 chains (SwissProt P07766), and one CD3 ζ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

A “BCMA×GPRC5D×CD3 antibody” is a trispecific antibody, which comprises three different antigen-binding arms, one of which binds to the antigen BCMA, one of which binds to the antigen GPRC5D, and one of which binds to CD3. The term “multispecific antibody” is used herein in the broadest sense and specifically covers an antibody that has polyepitopic specificity. Multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains where each V_{H}V_{L} unit binds to a different epitope, antibodies having two or more single variable domains with each single variable domain binding to a different epitope, full length antibodies, and antibodies comprising one or more antibody fragments as well as antibodies comprising antibody fragments that have been linked covalently or non-covalently.

A multispecific antibody can be a bispecific antibody, a trispecific antibody, diabody, or similar molecule (see for instance PNAS USA 90 (14), 6444-8 (1993) for a description of diabodies). The bispecific antibodies, trispecific antibodies, diabodies, and the like, provided herein may bind any suitable target in addition to a portion of BCMA or GPRC5D. The term “bispecific antibody” is to be understood as an antibody having two different antigen-binding arms defined by different antibody sequences. The term “trispecific antibody” is to be understood as an antibody having three different antigen-binding arms defined by different antibody sequences. This can be understood as different target binding but includes as well binding to different epitopes in one target.

The term “humanized antibody” refers to an antibody in which at least one CDR is derived from non-human species and at least one framework is derived from human immunoglobulin sequences. Humanized antibody can include substitutions in the frameworks so that the frameworks can not be exact copies of expressed human immunoglobulin or human immunoglobulin germline gene sequences.

A “reference sample” is a sample that may be compared against another sample, such as a test sample, to allow for characterization of the compared sample. The reference sample will have some characterized property that serves as the basis for comparison with the test sample. For instance, a reference sample may be used as a benchmark for GPRC5D or BCMA levels that are indicative of a subject having cancer. The reference sample does not necessarily have to be analyzed in parallel with the test sample, thus in some instances the reference sample may be a numerical value or range previously determined to characterize a given condition, such as GPRC5D or BCMA levels that are indicative of cancer in a subject. The term also includes samples used for comparative purposes that are known to be associated with a physiologic state or disease condition, such as GPRC5D- or BCMA-expressing cancer, but that have an unknown amount of GPRC5D or BCMA.

“Cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

“Combination” means that two or more therapeutics are administered to a subject, concurrently as single agents or sequentially as single agents in any order.

“Flat dose” refers to a dose that is administered to a subject without correction for the subject's specific body weight or body surface area. A flat dose, sometimes referred to as a fixed dose, is therefore provided as an absolute amount of the agent (e.g., mg drug), and not as a weight-based amount that accounts for the subject's specific weight (e.g. μg/kg or μg drug per kg body weight). For example, a subject weighing 65 kg may be administered the same flat dose in milligrams as a subject weighing 85 kg. A flat dose may be administered according to a pre-defined class or category of body weight, but is not modified according to the subject's specific weight. For example, a “Flat Dose A” may be administered if a patient is greater than a pre-defined threshold weight, whereas a different “Flat Dose B” may be administered if the patent is less than the pre-defined threshold weight.

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Newly diagnosed” or “newly diagnosed multiple myeloma” refers to a clinical status in which a patient has been diagnosed with multiple myeloma for the first time and has not yet received any systemic therapy for the disease, or has received only limited initial treatment (such as corticosteroids or a single cycle of chemotherapy) not intended as full induction therapy. This term encompasses patients at the time of diagnosis prior to initiation of a standard treatment regimen and may include those who are transplant-eligible or transplant-ineligible.

The term “progression,” as used in the context of progression of GPRC5D and/or BCMA-expressing cancer, includes the change of a cancer from a less severe to a more severe state. This may include an increase in the number or severity of tumors, the degree of metastasis, the speed with which the cancer is growing or spreading, and the like. For example, “the progression of colon cancer” includes the progression of such a cancer from a less severe to a more severe state, such as the progression from stage I to stage II, from stage II to stage III, etc.

The term “regression,” as used in the context of regression of GPRC5D and/or BCMA-expressing cancer, includes the change of a cancer from a more severe to a less severe state. This could include a decrease in the number or severity of tumors, the degree of metastasis, the speed with which the cancer is growing or spreading, and the like. For example, “the regression of colon cancer” includes the regression of such a cancer from a more severe to a less severe state, such as the progression from stage III to stage II, from stage II to stage I, etc.

The term “stable” as used in the context of stable GPRC5D and/or BCMA-expressing cancer, is intended to describe a disease condition that is not, or has not, changed significantly enough over a clinically relevant period of time to be considered a progressing cancer or a regressing cancer.

The term “step-up dose” refers to a dose of an active agent that is administered to a subject prior to a treatment dose. A step-up dose is lower than the treatment dose. To prevent or lessen certain toxicities, such as cytokine release syndrome (CRS), a “priming” dose strategy may include one or more lower step-up dose(s) followed by higher treatment doses.

The term “treatment dose” refers to a dose of the active agent that is administered to a subject to treat a disease. A treatment dose may be administered at a regular dosing interval on a repetitive basis (e.g. weekly, biweekly, monthly). A treatment dose may be preceded by one or more step-up doses.

As used herein, the term “outpatient” is a patient who attends a hospital or clinic for treatment without staying at the hospital or clinic overnight, and the patient is administered the BCMA×GPRC5D×CD3 trispecific antibody or trispecific binding fragment thereof described herein by a health care professional.

The term “high-risk cytogenetic abnormalities” described herein refers to genetic alterations in multiple myeloma cells that are associated with poor prognosis, increased disease aggressiveness, or resistance to standard therapies, including but not limited to deletions, translocations, or amplifications such as del(17p), t(4;14), t(14;16), or 1q21 gain/1q21 amplification, as identified through methods such as fluorescence in situ hybridization (FISH), next-generation sequencing (NGS), or equivalent cytogenetic analysis techniques.

The embodiments described herein are not limited to particular methods, reagents, compounds, compositions or biological systems, which can, of course, vary.

### BCMA×GPRC5D×CD3 Trispecific Antibodies

Any suitable BCMA×GPRC5D×CD3 trispecific antibody or trispecific binding fragment thereof known to those skilled in the art in view of the present disclosure can be used.

The methods provided herein comprise administering trispecific antibodies that bind to BCMA, GPRC5D, and CD3 (“BCMA×GPRC5D×CD3 trispecific antibodies”), or trispecific binding fragments thereof. Such antibodies or antibody fragments may allow for more specific targeting to particular subsets of cells as compared to antibodies targeting only one or two of these targets.

In some embodiments, the BCMA×GPRC5D×CD3 trispecific antibody or trispecific binding fragment thereof comprises any one of the BCMA binding domains described in WO2022/175255, the entire content of which is incorporated herein by reference. In some embodiments, the BCMA×GPRC5D×CD3 trispecific antibody or trispecific binding fragment thereof comprises any one of the GPRC5D binding domains described in WO2022/175255. In some embodiments, the BCMA×GPRC5D×CD3 trispecific antibody or trispecific binding fragment thereof comprises any one of the CD3 binding domains described in WO2022/175255. In some embodiments, the BCMA×GPRC5D×CD3 trispecific antibody comprises any one of the BCMA×GPRC5D×CD3 trispecific antibodies described in WO2022/175255.

The BCMA×GPRC5D×CD3 trispecific antibodies or binding fragments thereof can be provided by making a molecule which comprises a first antigen-binding arm binding to CD3, a second antigen-binding arm binding to GPRC5D and a third antigen-binding arm binding to the BCMA.

Accordingly, the methods comprise administering trispecific molecules comprising three different antigen-binding arms which bind BCMA, GPRC5D, and CD3, respectively.

In some embodiments, the BCMA×GPRC5D×CD3-trispecific antibody or trispecific binding fragment thereof comprises
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2); and
(c) a third antigen-binding arm comprising a third heavy chain variable domain (VH3) and a third light chain variable domain (VL3), wherein the first antigen-binding arm binds to an epitope on CD3, the second antigen-binding arm binds to an epitope on GPRC5D, and the third antigen-binding arm binds to an epitope on BCMA.

In some embodiments, the first antigen-binding arm that binds CD3 comprises a HCDR 1, a HCDR2 and a HCDR3 of the VH1 of SEQ ID NO: 8. In some embodiments, the first antigen-binding arm that binds CD3 comprises a LCDR1, a LCDR2 and a LCDR3 of the VL1 of SEQ ID NO: 7. In some embodiments, the first antigen-binding arm that binds CD3 comprises a HCDR1 comprising the amino acid sequence of GDSVENNNAAWS (SEQ ID NO: 4), a HCDR2 comprising the amino acid sequence of RTYYRSKWLYD (SEQ ID NO: 5), and a HCDR3 comprising the amino acid sequence of GYSSSFDY (SEQ ID NO: 6). In some embodiments, the first antigen-binding arm that binds CD3 comprises a LCDR1 comprising the amino acid sequence of TGTSSNIGTYKFVS (SEQ ID NO: 1), a LCDR2 comprising the amino acid sequence of EVSKRPS (SEQ ID NO: 2), and a LCDR3 comprising the amino acid sequence of VSYAGSGTLL (SEQ ID NO: 3). In some embodiments, the first antigen-binding arm that binds CD3 comprises the VH1 of SEQ ID NO: 8. In some embodiments, the first antigen-binding arm that binds CD3 comprises the VL1 of SEQ ID NO: 7.

In some embodiments, the second antigen-binding arm that binds GPRC5D comprises a HCDR1, a HCDR2 and a HCDR3 of the VH2 of SEQ ID NO: 16. In some embodiments, the second antigen-binding arm that binds GPRC5D comprises a LCDR1, a LCDR2 and a LCDR3 of the VL2 of SEQ ID NO: 15. In some embodiments, the second antigen-binding arm that binds GPRC5D comprises a HCDR1 comprising the amino acid sequence of GFSLTNIRMSVS (SEQ ID NO: 12), HCDR2 comprising the amino acid sequence of HIFSNDEKS (SEQ ID NO: 13), and a HCDR3 comprising the amino acid sequence of MRLPYGMDV (SEQ ID NO: 14). In some embodiments, the second antigen-binding arm that binds GPRC5D comprises a LCDR1 comprising the amino acid sequence of RSSQSLVHSDGNTYLS (SEQ ID NO: 9), a LCDR2 comprising the amino acid sequence of KISNRFF (SEQ ID NO: 10), and a LCDR3 comprising the amino acid sequence of MQATQFPHT (SEQ ID NO: 11). In some embodiments, the second antigen-binding arm that binds GPRC5D comprises the VH1 of SEQ ID NO: 16. In some embodiments, the second antigen-binding arm that binds GPRC5D comprises the VL1 of SEQ ID NO: 15.

In some embodiments, the third antigen-binding arm that binds BCMA comprises a HCDR1, a HCDR2 and a HCDR3 of the VH3 of SEQ ID NO: 24. In some embodiments, the third antigen-binding arm that binds BCMA comprises a LCDR1, a LCDR2 and a LCDR3 of the VL3 of SEQ ID NO: 23. In some embodiments, the third antigen-binding arm that binds BCMA comprises a HCDR1 comprising the amino acid sequence of GFTFSSYAMS (SEQ ID NO: 20), a HCDR2 comprising the amino acid sequence of AISGSGGSTY (SEQ ID NO: 21), and a HCDR3 comprising the amino acid sequence of DEGYSSGHYYGMDV (SEQ ID NO: 22); and a LCDR1 comprising the amino acid sequence of RASQSISSSFLT (SEQ ID NO: 17). In some embodiments, the third antigen-binding arm that binds BCMA comprises a LCDR2 comprising the amino acid sequence of GASSRAT (SEQ ID NO: 18), and a LCDR3 comprising the amino acid sequence of QHYGSSPMYT (SEQ ID NO: 19). In some embodiments, the third antigen-binding arm that binds BCMA comprises the VH1 of SEQ ID NO: 24. In some embodiments, the third antigen-binding arm that binds BCMA comprises the VL1 of SEQ ID NO: 23.

In some embodiments, the VH1 and VL1 of the antigen-binding arm that binds to CD3 epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the VH2 and VL2 of the antigen-binding arm that binds to GPRC5D epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the VH3 and VL3 of the antigen-binding arm that binds to BCMA epitope are present in a diabody, a Fab, Fab′, a F(ab′)2, a Fv, a scFv, a Fd, a disulfide stabilized Fv fragment (dsFv), or a disulfide stabilized diabody (ds diabody).

In one embodiment, the CD3-binding arm comprises an antigen-binding fragment (Fab), the BCMA-binding arm comprises a single-chain variable fragment (scFv), and the GPRC5D-binding arm comprises a single-chain variable fragment (scFv).

In some embodiments, the CD3-binding arm of the trispecific antibody comprises the HC1 and the LC. The HC1 may comprise constant heavy chain regions (CH1, CH2, and CH3) and the VH1. The LC may comprise the VL1. The VH1 and VL1 combine to form the CD3 antigen binding domain.

In some embodiments, the BCMA-binding arm of the trispecific antibody comprises the HC2. The HC2 may comprise constant heavy chain regions (CH2 and CH3), and a single-chain variable fragment (scFv) attached at the N-terminus of the CH2 region, wherein the scFv comprises the BCMA antigen binding domain.

In some embodiments, the trispecific antibody further comprises GPRC5D antigen-binding arm attached to the C-terminus of the CH3 region of the CD3-binding arm to form a CD3/GPRC5D binding arm. In some embodiments, the BCMA antigen-binding arm comprises a second single-chain variable fragment (scFv). In some embodiments, the CD3/GPRC5D arm may have the structure: Fab containing the CD3 binding domain, CH2 and CH3 regions, scFv containing the GPRC5D binding domain.

In some embodiments, the trispecific antibodies of the disclosure include antibodies having a full length antibody structure. “Full length antibody” as used herein refers to an antibody having two full length antibody heavy chains and two full length antibody light chains. A full length antibody heavy chain (HC) includes heavy chain variable and constant domains VH, CH1, CH2, and CH3. A full length antibody light chain (LC) includes light chain variable and constant domains VL and CL. The full length antibody may be lacking the C-terminal lysine (K) in either one or both heavy chains. The term “Fab-arm” or “half molecule” refers to one heavy chain-light chain pair that binds an antigen.

### BCMA-Binding Arm

The BCMA×GPRC5D×CD3 trispecific antibody described herein comprises an antigen-binding arm specific for BCMA. In some embodiments, the BCMA-binding arm binds human BCMA. In some embodiments, the BCMA-binding arm binds to residues 17-26 (LLHACIPCQL (SEQ ID NO: 162)) of BCMA BCMW37 chain.

Characteristics of some BCMA-specific antibodies or antigen-binding fragments may be found in e.g., WO2022/175255, the content of which is herein incorporated by reference in its entirety.

In one embodiment, the BCMA-binding arm comprises heavy chain CDR1, CDR2, and CDR3 and light chain CDR1, CDR2, and CDR3 of clone BCMB519. In one embodiment, the BCMA-binding arm comprises heavy chain variable domain and light chain variable domain of clone BCMB519.

In some embodiments, the BCMA-binding arm comprises a heavy chain CDR1 comprising SEQ ID NO: 20, a heavy chain CDR2 comprising SEQ ID NO: 21, and a heavy chain CDR3 comprising SEQ ID NO: 22. In some embodiments, the BCMA-binding arm comprises a heavy chain CDR1 comprising SEQ ID NO: 20, a heavy chain CDR2 comprising SEQ ID NO: 21, a heavy chain CDR3 comprising SEQ ID NO: 22, a light chain CDR1 comprising SEQ ID NO: 17, a light chain CDR2 comprising SEQ ID NO: 18, and a light chain CDR3 comprising SEQ ID NO: 19. The BCMA-binding arm may comprise human framework sequences. In some embodiments, the BCMA-binding arm comprises a heavy chain variable domain substantially the same as, or identical to, SEQ ID NO: 24. In some embodiments, the BCMA-binding arm comprises a heavy chain variable domain substantially the same as, or identical to, SEQ ID NO: 24 and a light chain variable domain substantially the same as, or identical to, SEQ ID NO: 23.

In some embodiments, the BCMA-binding arm comprises humanized antigen-binding fragments. Humanized antigen-binding fragments may be derived from chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies or antigen-binding fragments are human immunoglobulins (recipient antibody) or antigen-binding fragments in which residues from a complementary-determining region (CDR) of the recipient are replaced by residues from a CDR of a non-human species (donor antibody) such as mouse, rat or rabbit having the desired specificity, affinity, and capacity. In general, the humanized antibody antigen-binding fragments will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody antigen-binding fragments may include at least a portion of an immunoglobulin constant region (Fc), typically that of a human immunoglobulin.

### GPRC5D-Binding Arm

The BCMA×GPRC5D×CD3-trispecific antibody or trispecific binding fragment thereof described herein comprises an antigen-binding arm specific for GPRC5D.

In some embodiments, the GPRC5D-binding arm binds human GPRC5D. In some embodiments, the GPRC5D-binding arm binds to one or more residues of a polypeptide having the amino acid sequence of SEQ ID NO: 116.

Characteristics of some GPRC5D-specific antibodies or antigen-binding fragments may be found in e.g., United States Published Application US2020/0231686, the content of which is herein incorporated by reference in its entirety.

In one embodiment, the GPRC5D-binding arm comprises heavy chain CDR1, CDR2, and CDR3 and light chain CDR1, CDR2, and CDR3 of clone GC5B680. In one embodiment, the GPRC5D-binding arm comprises heavy chain variable domain and light chain variable domain of clone GC5B680.

In some embodiments, the GPRC5D-binding arm comprises a heavy chain CDR1 comprising SEQ ID NO: 12, a heavy chain CDR2 comprising SEQ ID NO: 13, and a heavy chain CDR3 comprising SEQ ID NO: 14. In some embodiments, the GPRC5D-binding arm comprises a heavy chain CDR1 comprising SEQ ID NO: 12, a heavy chain CDR2 comprising SEQ ID NO: 13, a heavy chain CDR3 comprising SEQ ID NO: 14, a light chain CDR1 comprising SEQ ID NO: 9, a light chain CDR2 comprising SEQ ID NO: 10, and a light chain CDR3 comprising SEQ ID NO: 11. The GPRC5D-binding arm may comprise human framework sequences. In some embodiments, the GPRC5D-binding arm comprises a heavy chain variable domain substantially the same as, or identical to, SEQ ID NO: 16. In some embodiments, the GPRC5D-binding arm comprises a heavy chain variable domain substantially the same as, or identical to, SEQ ID NO: 16 and a light chain variable domain substantially the same as, or identical to, SEQ ID NO: 15.

### CD3-Binding Arm

The BCMA×GPRC5D×CD3-trispecific antibody or trispecific binding fragment thereof described herein comprises an antigen-binding arm that binds CD3. In some embodiments, the CD3-binding arm binds human CD3. In some embodiments, the CD3-specific arm of the BCMA×GPRC5D×CD3 trispecific antibody is derived from a CD3-specific antibody that binds and activates human primary T cells and/or cynomolgus monkey primary T cells. In some embodiments, the trispecific antibodies or trispecific antigen-binding fragments described herein bind to CD3ε. In some embodiments, the CD3-binding arm binds to an epitope at the N-terminus of CD3ε. In some embodiments, the CD3-binding arm binds to residues 22-35 (QDGNEEMGGITQTP (SEQ ID NO: 161)) of the CD3ε chain.

Human CD3ε is described under UniProt P07766 (CD3E_HUMAN). An anti CD3& antibody described in the state of the art is SP34 (Yang S J, The Journal of Immunology (1986) 137; 1097-1100). SP34 reacts with both primate and human CD3. SP34 is available from Pharmingen. A further anti CD3 antibody described in the state of the art is UCHT-1 (see WO2000041474). A further anti-CD3 antibody described in the state of the art is BC-3 (Fred Hutchinson Cancer Research Institute; used in Phase I/II trials of GvHD, Anasetti et al., Transplantation 54:844 (1992)). SP34 differs from UCHT-1 and BC-3 in that SP-34 recognizes an epitope present on solely the & chain of CD3 (see Salmeron et al., (1991) J. Immunol. 147:3047) whereas UCHT-1 and BC-3 recognize an epitope contributed by both the ε and γ chains. The sequence of an antibody with the same sequence as of antibody SP34 is mentioned in WO2008119565, WO2008119566, WO2008119567, WO2010037836, WO2010037837 and WO2010037838. A sequence which is 96% identical to VH of antibody SP34 is mentioned in U.S. Pat. No. 8,236,308 (WO2007042261).

Characteristics of some CD3-specific antibodies or antigen-binding fragments may be found in e.g., WO2019/224717, U.S. Pat. No. 11,603,405, United States Published Application US2023/322924, the content of each of which is herein incorporated by reference in its entirety.

In some embodiments, the CD3-binding arm (or “CD3-specific arm”) of the BCMA×GPRC5D×CD3 trispecific antibody or trispecific binding fragment thereof is derived from the monoclonal antibody CD3B376. In some embodiments, the CD3-binding arm comprises heavy chain CDR1, CDR2, and CDR3 and light chain CDR1, CDR2, and CDR3 of clone CD3B376.

In some embodiments, the CD3-binding arm comprises a heavy chain CDR1 comprising SEQ ID NO: 4, a heavy chain CDR2 comprising SEQ ID NO: 5, and a heavy chain CDR3 comprising SEQ ID NO: 6. In some embodiments, the CD3-binding arm comprises a heavy chain CDR1 comprising SEQ ID NO: 4, a heavy chain CDR2 comprising SEQ ID NO: 5, a heavy chain CDR3 comprising SEQ ID NO: 6, a light chain CDR1 comprising SEQ ID NO: 1, a light chain CDR2 comprising SEQ ID NO: 2, and a light chain CDR3 comprising SEQ ID NO: 3. The CD3-binding arm may comprise human framework sequences. In some embodiments, the CD3-binding arm comprises a heavy chain variable domain substantially the same as, or identical to, SEQ ID NO: 8. In some embodiments, the CD3-binding arm comprises a heavy chain variable domain substantially the same as, or identical to, SEQ ID NO: 8 and a light chain variable domain substantially the same as, or identical to, SEQ ID NO: 7.

In some embodiments, the CD3-binding arm is IgG, or a derivative thereof. In some embodiments, the CD3-specific antibody or antigen-binding fragment from which the CD3-specific arm of the trispecific antibody is derived is IgG1, or a derivative thereof. In some embodiments, for example, the Fc region of the CD3-specific IgG1 antibody from which the CD3-binding arm is derived comprises L234A, L235A, and D265S substitutions in its Fc region.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Inti. Publ. No. WO 2006/028936) may be used to generate full length trispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody binding a first antigen and an amino acid with a large side chain (knob) is introduced into a heavy chain of an antibody binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

In some embodiments of the trispecific antibody or trispecific binding fragment described herein, one of the Fc domains comprise mutations T366S, L368A and Y407V and the other Fc domain comprises mutation T366W. In some embodiments, the Fc domain of the first heavy chain portion (HC1) of the first antigen binding arm (e.g., CD3 binding arm) comprises mutations T366S, L368A and Y407V, and the Fc domain of the second heavy chain portion (HC2) of the third antigen-binding arm (e.g., the BCMA binding arm) comprises mutation T366W.

In some embodiments, the trispecific antibodies or antigen-binding fragments are IgG, or derivatives thereof.

In some embodiments wherein the antibody is of IgG1 isotype, the antibody comprises an IgG1 Fc region (SEQ ID NO: 158).

SEQ ID NO: 158
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments wherein the antibody is of IgG1 isotype, the antibody comprises L234A, L235A, and D265S substitutions (underlined) in its Fc region (SEQ ID NO: 159).

SEQ ID NO: 159
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
SVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments wherein the antibody has an IgG1 isotype, the antibody contains L234A, L235A, D265S and/or K409R substitution(s) in its Fc region. The antibodies described herein may include these modifications.

In some embodiments, the Fc domains of a trispecific antibody described herein each comprise one or more mutations selected from L234A, L235A, and D265S. In some embodiments, the Fc domains of HC1 and HC2 each comprise mutations L234A, L235A, and D265S.

In some embodiments, the Fc domain of one of the heavy chain portions of a trispecific antibody described herein further comprise one or more mutations which reduce Fc binding to protein A. In some embodiments, the Fc domain of the HC1 comprises mutations H435R and/or Y436F, preferably H435R and Y436F.

In some embodiments of a trispecific antibody described herein, the HC1 comprises, from the N- to C-terminus, the VH1 of the first antigen-binding arm, a CH1 domain, the Fc domain, a linker, and the second antigen-binding arm.

In various embodiments, the scFvs used in trispecific antibodies or trispecific binding fragments described herein comprise, from the N- to C-terminus, a VH, a linker and a VL (VH-L-VL) or the VL, the linker and the VH (VL-L-VH). In some embodiments, the scFv comprises, from the N- to C-terminus, the VL, the linker and the VH (VL-L-VH).

In some embodiments, the linker comprises the amino acid sequence of SEQ ID NO: 25.

In some embodiments, the linker comprises the amino acid sequence of SEQ ID NO: 163.

In one embodiment, a CD3/GPRC5D coupled HC1 of a BCMA×GPRC5D×CD3 trispecific antibody comprises an amino acid sequence substantially the same as, or identical to, SEQ ID NO: 29.

In one embodiment, the LC of a BCMA×GPRC5D×CD3 trispecific antibody comprises an amino acid sequence substantially the same as, or identical to, SEQ ID NO: 30.

In one embodiment, the BCMA binding arm (HC2) comprises an amino acid sequence substantially the same as, or identical to, SEQ ID NO: 31.

In one embodiment, provided herein is an isolated trispecific antibody, or a trispecific binding fragment thereof, comprising:
a) a CD3 binding arm comprising a heavy chain (HC1) and a light chain (LC), wherein the HC1 further comprises the GPRC5D binding arm; and
b) a BCMA binding arm,
wherein HC1 comprises an amino acid sequence substantially the same as, or identical to, SEQ ID NO: 29, LC comprises an amino acid sequence substantially the same as, or identical to, SEQ ID NO: 30, and the BCMA binding arm comprises an amino acid sequence substantially the same as, or identical to, SEQ ID NO: 31.

In one embodiment, provided herein is an isolated trispecific antibody, or a trispecific binding fragment thereof, comprising:
a) a CD3 binding arm comprising a heavy chain (HC1) and a light chain (LC), wherein the HC1 further comprises the GPRC5D binding arm; and
b) a BCMA binding arm,
wherein HC1 comprises the amino acid sequence of SEQ ID NO: 29, LC comprises the amino acid sequence of SEQ ID NO: 30, and the BCMA binding arm comprises the amino acid sequence of SEQ ID NO: 31.

In one embodiment, the BCMA×GPRC5D×CD3 trispecific antibody comprises:
(a) a HC1 comprising the amino acid sequence of SEQ ID NO: 29; (b) an LC comprising the amino acid sequence of SEQ ID NO: 30, and (c) a HC2 comprising an amino acid sequence of SEQ ID NO: 31. In one embodiment, the BCMA×GPRC5D×CD3 trispecific antibody is BGCB491. In one embodiment, BGCB491 comprises:
(a) a HC1 comprising the amino acid sequence of SEQ ID NO: 29; (b) an LC comprising the amino acid sequence of SEQ ID NO: 30, and (c) a HC2 comprising an amino acid sequence of SEQ ID NO: 31.

### Anti-CD38 Antibody

In one embodiment, the anti-CD 38 antibody comprises a heavy chain complementarity determining region 1 (HCDR1) of SEQ ID NO: 201, a HCDR2 of SEQ ID NO: 202, a HCDR3 of SEQ ID NO: 203, a light chain complementarity determining region 1 (LCDR1) of SEQ ID NO: 204, a LCDR2 of SEQ ID NO: 205 and a LCDR3 of

SEQ ID NO: 206.

In certain embodiments, the anti-CD 38 antibody comprises a heavy chain variable region (VH) of SEQ ID NO: 207 and a light chain variable region (VL) of SEQ ID NO: 208. In further embodiments, the anti-CD 38 antibody is an IgG1 isotype. In still further embodiments, the anti-CD38 antibody comprises a heavy chain (HC) of SEQ ID NO: 209 and a light chain (LC) of SEQ ID NO: 210. An exemplary IgG1 constant domain sequence comprises an amino acid sequence of SEQ ID NO: 211. Some variation exists within the IgG1 constant domain (e.g., well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28

In one embodiment, the anti-CD 38 antibody is daratumumab. In a further embodiment, the anti-CD 38 antibody is formulated in a pharmaceutical composition. In a further embodiment, the anti-CD 38 antibody is a marketed product containing daratumumab (such as Darzalex®, Darzalex SC®, Darzalex Faspro®), or a biosimilar of daratumumab or a biosimilar of a marketed product containing daratumumab.

In further embodiments of any of the methods disclosed herein, the anti-CD38 antibody contains daratumumab, and is formulated in a pharmaceutical composition. In such an embodiment, the pharmaceutical composition may further contain a hyaluronidase; in an embodiment the pharmaceutical embodiment comprises a hyaluronidase that is recombinant human hyaluronidase (rHuPH20).

A number of suitable hyaluronidase enzymes in accordance with the present disclosure are known. The enzyme is, in an embodiment, a human hyaluronidase enzyme, such as a soluble human PH20 hyaluronidase, in an embodiment, the recombinant human hyaluronidase enzyme product known as rHuPH20. The form of the hyaluronidase as defined herein is the form that is combined with the antibody in the pharmaceutical formulation. The amino acid sequence of soluble human PH20 hyaluronidases include the soluble human PH20 known as rHuPH20 and available under CAS Registry No. 757971-58-7. In some embodiments, rHuPH20 is Hylenex®. Soluble human PH20 hyaluronidases are described in Int. Pat. Publ. No. WO2004/078140 and U.S. Pat. No. 7,767,429 incorporated herein by reference, in its entirety. In some embodiments, soluble hyaluronidases include those whose sequence are set forth in any of SEQ ID NOs: 212-216. Soluble PH20 hyaluronidase, when expressed in a cell, include a signal sequence for trafficking in the cell. In some embodiments, the amino acid sequence is the sequence of a soluble PH20 hyaluronidase SEQ ID NO: 213, namely residues 36-482 of wild type human hyaluronidase. In some embodiments, the amino acid sequence of a soluble PH20 hyaluronidase comprises SEQ ID NO: 214. In some embodiments, the amino acid sequence of a soluble PH20 hyaluronidase comprises SEQ ID NO: 215. In some embodiments, the amino acid sequence a soluble PH20 hyaluronidase rHuPH20 comprises SEQ ID NO: 216. In some embodiments, the amino acid sequence of a soluble PH20 hyaluronidase comprises SEQ ID NO: 212. In some embodiments, the soluble PH20 hyaluronidases, when expressed in a cell, comprise a mixture of species that can include any one or more of SEQ ID NO: 212 to SEQ ID NO: 216 in various abundance. The average molecular weight is 61 kDa.

rHuPH20 refers to the composition produced upon expression in a cell, such as CHO cell. As produced in the culture medium there is heterogeneity at the C-terminus such that the product, designated rHuPH20, includes a mixture of species that can include any one or more of the polypeptides of SEQ ID NO: 12-16, and some shorter polypeptides, in various abundance. Typically, rHuPH20 is produced in cells, such as CHO cells, (for example DG44 CHO cells) that facilitate correct N-glycosylation to retain activity. In an embodiment of any of the methods disclosed herein, the anti-CD 38 antibody

or the pharmaceutical composition containing the anti-CD 38 antibody is administered subcutaneously.

In an embodiment of any of the methods disclosed herein, daratumumab is formulated in a pharmaceutical composition comprising 1800 mg daratumumab and 30000 units hyaluronidase.

In an embodiment of any of the methods disclosed herein, daratumumab is formulated in a pharmaceutical composition comprising 1800 mg daratumumab and 30000 units hyaluronidase and is administered subcutaneously.

### Lenalidomide

Lenalidomide is a potent immunomodulatory drug (IMiD) chemically designated as (E)-3-(4-amino-1,2-dihydro-2-oxo-1H-pyrrole-1-carboxamido)-2-methylprop-2-enoic acid, with a molecular formula of C₁₃H₁₃N₃O₃C₁₃H₁₃N₃O₃. As a thalidomide analogue, it possesses a unique chemical structure that includes a pyrrole ring, contributing to its therapeutic properties. Lenalidomide operates through multiple mechanisms, primarily enhancing the immune system's ability to detect and eradicate malignant cells. It promotes T cell proliferation, augments natural killer (NK) cell activity, and induces apoptosis in myeloma cells via the activation of intracellular signaling pathways. Additionally, lenalidomide has anti-angiogenic properties, inhibiting the production of pro-angiogenic factors and thus reducing the vascular support for tumors. It also modulates the secretion of various inflammatory cytokines, including tumor necrosis factor-alpha (TNF-α) and interleukin-6 (IL-6), which are implicated in the pathogenesis of multiple myeloma.

Clinically, lenalidomide has been approved for the treatment of multiple myeloma in several contexts, including its use as a monotherapy for patients who have received at least one prior treatment and as part of combination therapies, particularly with dexamethasone or proteasome inhibitors like bortezomib. The drug is typically administered at an initial oral dose of approximately 25 mg once daily for 21 days, followed by a 7-day rest period, resulting in a 28-day treatment cycle, with dosing adjustments based on patient tolerance and any observed hematologic toxicity.

Lenalidomide, marketed under the trademark Revlimid®, has received regulatory approval from the U.S. Food and Drug Administration (FDA) and is recognized as part of the therapeutic landscape for multiple myeloma.

### Dexamethasone

Dexamethasone is a synthetic adrenocortical steroid with potent glucocorticoid activity and minimal mineralocorticoid activity. It exerts broad anti-inflammatory and immunosuppressive effects by binding to cytosolic glucocorticoid receptors and modulating the transcription of corticosteroid-responsive genes involved in immune cell proliferation, cytokine production, and inflammatory mediator release.

Clinically, dexamethasone is used to treat a wide range of inflammatory, allergic, and autoimmune disorders, including rheumatoid arthritis, systemic lupus erythematosus, asthma, and bronchospasm. It also exerts cytotoxic and apoptotic effects on certain hematologic malignancies, thereby serving as a chemotherapeutic agent in various oncologic regimens.

In the context of hematologic cancers such as multiple myeloma, dexamethasone is routinely administered as a component of multi-drug regimens to potentiate the anti-tumor effects of immunomodulatory agents (e.g., lenalidomide or pomalidomide), proteasome inhibitors (e.g., bortezomib or carfilzomib), and monoclonal or multispecific antibodies (e.g., BCMA- or GPRC5D-targeted antibodies). The glucocorticoid reduces tumor-associated inflammation, mitigates cytokine-mediated adverse events, and induces apoptosis in plasma cells, thereby contributing directly to anti-myeloma activity and improving tolerability of combination therapies.

Accordingly, dexamethasone may be administered alone or in combination with one or more chemotherapeutic, immunomodulatory, or antibody-based agents described herein, including but not limited to trispecific antibodies targeting BCMA×GPRC5D×CD3. When used in combination therapy, dexamethasone may be administered prior to, concurrently with, or subsequent to administration of the antibody or antibody fragment, at dosages effective to enhance therapeutic efficacy and reduce immune-related toxicity.

### Treatment of Multiple Myeloma

The trispecific antibodies discussed above, for example the BCMA×GPRC5D×CD3 trispecific antibodies discussed above, are for use in treating multiple myeloma (MM).

Similarly, the anti-CD38 antibody discussed above, for example daratumumab, are for use in treating multiple myeloma (MM).

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is administered at a treatment dose of at least about 20 mg.

In one aspect, provided herein is a method of treating multiple myeloma in a subject in need thereof, comprising administering a therapeutically effective amount of a BCMA×GPRC5D×CD3 trispecific antibody or a trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of at least about 20 mg.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 20 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 50 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 100 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 200 mg and wherein the treatment dose is administered once every four weeks.

In another aspect, provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody, to the subject to treat the multiple myeloma, wherein the BCMA×GPRC5D×CD3 trispecific antibody or trispecific binding fragment thereof is subcutaneously administered at a treatment dose of about 300 mg and wherein the treatment dose is administered once every four weeks.

### Administration

In particular, the disclosure provides new regimens for administering a BCMA×GPRC5D×CD3 trispecific antibodies or trispecific fragments thereof and an anti-CD38 antibody to treat multiple myeloma in a subject.

### Dosing

According to a particular aspect, the BCMA×GPRC5D×CD3 trispecific antibody or trispecific binding fragment thereof is administered to a subject at a treatment dose of at least about 20 mg.

In some embodiments, the treatment dose is at least about 20 mg, at least about 50 mg, at least about 100 mg, at least about 200 mg or at least about 300 mg.

In one embodiment, the treatment dose is at least about 20 mg. In another embodiment, the treatment dose is at least about 50 mg. In an embodiment, the treatment dose is at least about 100 mg. In another embodiment, the treatment dose is at least about 200 mg. In yet another embodiment, the treatment dose is at least about 300 mg.

In some embodiments, the treatment dose of the BCMA×GPRC5D×CD3 trispecific antibody or trispecific binding fragment thereof is about 20 mg to about 300 mg, about 20 mg to about 200 mg, about 20 mg to about 100 mg, about 20 mg to about 50 mg, about 50 mg to about 300 mg, about 50 mg to about 100 mg, about 100 mg to about 300 mg,

The treatment dose of the BCMA×GPRC5D×CD3 trispecific antibody or trispecific binding fragment thereof may be a flat dose, as defined herein. Flat dosing may provide advantages compared to dosing according to body weight, such as reduced preparation time, and simpler administration and manufacturing. In some embodiments, the treatment dose is a flat dose.

For example, in some embodiments, the treatment dose is a flat dose of at least about 20 mg, at least about 50 mg, at least about 100 mg, at least about 200 mg, or at least about 300 mg.

In some embodiments, the treatment dose is a flat dose within the range of about 20 mg to about 300 mg, about 20 mg to about 200 mg, about 20 mg to about 100 mg, about 20 mg to about 50 mg, about 50 mg to about 300 mg, about 50 mg to about 100 mg, about 100 mg to about 300 mg.

In certain embodiments, the treatment dose is a flat dose of about 20 mg, about 50 mg, about 100 mg, about 200 mg, about 300 mg. In one embodiment, the treatment dose is a flat dose of about 20 mg. In one embodiment, the treatment dose is a flat dose of about 50 mg. In one embodiment, the treatment dose is a flat dose of about 100 mg. In one embodiment, the treatment dose is a flat dose of about 200 mg. In one embodiment, the treatment dose is a flat dose of about 300 mg.

The treatment dose of the BCMA×GPRC5D×CD3 trispecific antibody or trispecific binding fragment thereof may be a weight-based dose. Therefore, in some embodiments, the treatment dose is a weight-based dose. Such dosages can, for example, be based on the mg/kg dosages provided above according to the following: dose (mg/kg)×body weight (e.g., 50-100 kg).

The treatment dose of the anti-CD38 antibody may be a flat dose, as defined herein. Flat dosing may provide advantages compared to dosing according to body weight, such as reduced preparation time, and simpler administration and manufacturing. In some embodiments, the treatment dose is a flat dose.

In an embodiment of any of the methods disclosed herein, the anti-CD38 antibody comprises a heavy chain complementarity determining region 1 (HCDR1) of SEQ ID NO: 201, a HCDR2 of SEQ ID NO: 202, a HCDR3 of SEQ ID NO: 203, a light chain complementarity determining region 1 (LCDR1) of SEQ ID NO: 204, a LCDR2 of SEQ ID NO: 205 and a LCDR3 of SEQ ID NO: 206._In certain embodiments, the anti-CD 38 antibody comprises a heavy chain variable region (VH) of SEQ ID NO: 207 and a light chain variable region (VL) of SEQ ID NO: 208. In further embodiments, the anti-CD 38 antibody is an IgG1 isotype. In still further embodiments, the anti-CD38 antibody comprises a heavy chain (HC) of SEQ ID NO: 209 and a light chain (LC) of SEQ ID NO: 210. An exemplary IgG1 constant domain sequence comprises an amino acid sequence of SEQ ID NO: 211. Some variation exists within the IgG1 constant domain (e.g., well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28. In an embodiment, the anti-CD 38 antibody is daratumumab. In a further embodiment, the anti-CD 38 antibody is formulated in a pharmaceutical composition. In a further embodiment, the anti-CD 38 antibody is a marketed product containing daratumumab (such as Darzalex®, Darzalex SC®, Darzalex Faspro®), or a biosimilar of daratumumab or a biosimilar of a marketed product containing daratumumab. In an embodiment of any of the methods disclosed herein, daratumumab is formulated in a pharmaceutical composition comprising 1800 mg daratumumab and 30000 units hyaluronidase.

### Administration Route

In some embodiments, the treatment dose of the BCMA×GPRC5D×CD3 trispecific antibody or trispecific binding fragment thereof is administered intravenously, intramuscularly, intraperitoneally, and/or subcutaneously to the subject. In some embodiments, the treatment dose is administered intravenously or subcutaneously to the subject. In some embodiments, the treatment dose is administered intravenously to the subject. In a further embodiment, the treatment dose is administered subcutaneously to the subject.

The administration may be performed by continuous infusion over a period of from 2 to 24 hr, such as from 2 to 12 hr. In one embodiment, the treatment dose of the BCMA×GPRC5D×CD3 trispecific antibody or trispecific binding fragment thereof may be administered by slow continuous infusion over a long period, such as more than 24 hours, in order to reduce toxic side effects. In some embodiments, the treatment dose of the BCMA×GPRC5D×CD3 trispecific antibody or trispecific binding fragment thereof is administered intravenously as a bolus or by continuous infusion over a period of time. In a further embodiment, the treatment dose of the BCMA×GPRC5D×CD3 trispecific antibody or trispecific binding fragment thereof is injected subcutaneously, to exert local as well as systemic therapeutic effects.

In some embodiments, the treatment dose of the anti-CD38 antibody is administered subcutaneously to the subject.

In an embodiment of any of the methods disclosed herein, the anti-CD38 antibody is daratumumab.

In an embodiment of any of the methods disclosed herein, daratumumab is formulated in a pharmaceutical composition comprising 1800 mg daratumumab and 30000 units hyaluronidase.

### Administration Frequency

In some embodiments, the treatment dose of the BCMA×GPRC5D×CD3 trispecific antibody or trispecific binding fragment thereof is administered once every four weeks.

In some embodiments the treatment dose of the BCMA×GPRC5D×CD3 trispecific antibody or trispecific binding fragment thereof is administered on Day 1 of at least one treatment cycle of every four weeks or about 28 days (e.g. a 28-day cycle).

The administration of the BCMA×GPRC5D×CD3 trispecific antibody or trispecific binding fragment thereof once every 4 weeks can therefore also be understood to start a treatment cycle, which can also here be referred to as a “BCMA×GPRC5D×CD3 trispecific antibody or trispecific binding fragment thereof treatment cycle”.

In some embodiments the treatment dose of the BCMA×GPRC5D×CD3 trispecific antibody or trispecific binding fragment thereof is administered on Day 1 of each BCMA×GPRC5D×CD3 trispecific antibody or trispecific binding fragment thereof treatment cycle.

In some embodiments the duration of the BCMA×GPRC5D×CD3 trispecific antibody or trispecific binding fragment thereof treatment cycle is about 28 days (e.g. a 28-day cycle).

For the purpose of sequencing of the treatment events the BCMA×GPRC5D×CD3 trispecific antibody or trispecific binding fragment thereof treatment cycles which can also be referred to as Cycle 1, Cycle 2, Cycle 3, Cycle 4 to signal their temporal occurrence, etc.

In some embodiments, the treatment dose of the anti-CD38 antibody is administered once every two weeks (biweekly). In some embodiments, the treatment dose of the anti-CD38 antibody is administered once every week (weekly). In some embodiments, the treatment dose of the anti-CD38 antibody administered once every four weeks.

In some embodiments, the treatment dose of the anti-CD38 antibody is administered during at least one BCMA×GPRC5D×CD3 trispecific antibody or trispecific binding fragment thereof treatment cycle. In certain such embodiments, the treatment dose of the anti-CD38 antibody is administered during at least one BCMA×GPRC5D×CD3 trispecific antibody or trispecific binding fragment thereof treatment cycle, beginning on Day 1 of Cycle 1.

In some embodiments the treatment dose of the anti-CD38 antibody is administered on Day 1 of at least one BCMA×GPRC5D×CD3 trispecific antibody or trispecific binding fragment thereof treatment cycle of every four weeks or about 28 days (e.g. a 28-day cycle).

In some embodiments, the treatment dose of the anti-CD38 antibody is administered during each BCMA×GPRC5D×CD3 trispecific antibody or trispecific binding fragment thereof treatment cycle. In certain such embodiments, the treatment dose of the anti-CD38 antibody is administered during each BCMA×GPRC5D×CD3 trispecific antibody or trispecific binding fragment thereof treatment cycle, beginning on Day 1 of Cycle 1.

In some embodiments the treatment dose of the anti-CD38 antibody is administered on Day 1 of each BCMA×GPRC5D×CD3 trispecific antibody or trispecific binding fragment thereof treatment cycle.

In some embodiments, the treatment dose of the anti-CD38 antibody is administered once every four weeks for each BCMA×GPRC5D×CD3 trispecific antibody or trispecific binding fragment thereof treatment cycle. In certain such embodiments, the treatment dose of the anti-CD38 antibody is administered on Day 1 of each BCMA×GPRC5D×CD3 trispecific antibody or trispecific binding fragment thereof treatment cycle. In certain such embodiments, the BCMA×GPRC5D×CD3 trispecific antibody or trispecific binding fragment thereof treatment cycle is a 28-day cycle.

In other embodiments, the treatment dose of the anti-CD38 antibody is administered once every week (weekly) for two BCMA×GPRC5D×CD3 trispecific antibody or trispecific binding fragment thereof treatment cycles (Cycle 1 and Cycle 2), followed by administration once every two weeks (biweekly) for four BCMA×GPRC5D×CD3 trispecific antibody or trispecific binding fragment thereof treatment cycles (Cycle 3, Cycle 4, Cycle 5 and Cycle 6), followed by administration once every four weeks for subsequent BCMA×GPRC5D×CD3 trispecific antibody or trispecific binding fragment thereof treatment cycles (≥Cycle 7). In certain such embodiments, the treatment dose of the anti-CD38 antibody is administered on Day 1, Day 8, Day 15, and Day 22 for two BCMA×GPRC5D×CD3 trispecific antibody or trispecific binding fragment thereof treatment cycles (Cycle 1 and Cycle 2); on Day 1 and Day 15 for four BCMA×GPRC5D×CD3 trispecific antibody or trispecific binding fragment thereof treatment cycles (Cycle 3, Cycle 4, Cycle 5 and Cycle 6); and on Day 1 for subsequent BCMA×GPRC5D×CD3 trispecific antibody or trispecific binding fragment thereof treatment cycles (≥Cycle 7). In certain such embodiments, the BCMA×GPRC5D×CD3 trispecific antibody or trispecific binding fragment thereof treatment cycle is a 28-day cycle.

In some embodiments, the treatment dose of the anti-CD38 antibody is administered once every week for two 28-day cycles, followed by administration once every two weeks for four 28-day cycles, followed by administration once every four weeks for subsequent 28-day cycles.

In some embodiments, the treatment dose of the anti-CD38 antibody is 1800 mg. In some embodiments, the anti-CD38 antibody is daratumumab. In certain such embodiments, the daratumumab is formulated in a pharmaceutical composition comprising 1800 mg daratumumab and 30000 units hyaluronidase.

In some embodiments, lenalidomide is administered during at least one BCMA×GPRC5D×CD3 trispecific antibody or trispecific binding fragment thereof treatment cycle.

In some embodiments, there is no lenalidomide administered during a BCMA×GPRC5D×CD3 trispecific antibody or trispecific binding fragment thereof treatment cycle. In certain such embodiments, no lenalidomide is administered during the first BCMA×GPRC5D×CD3 trispecific antibody or trispecific binding fragment thereof treatment cycle, or here also called Cycle 1.

In some embodiments, lenalidomide is administered during the second BCMA×GPRC5D×CD3 trispecific antibody or trispecific binding fragment thereof treatment cycle, or here also called Cycle 2.

In some embodiments, lenalidomide is administered during at least one BCMA×GPRC5D×CD3 trispecific antibody or trispecific binding fragment thereof treatment cycle, beginning on Day 1 of Cycle 2. In certain such embodiments, lenalidomide is administered during each BCMA×GPRC5D×CD3 trispecific antibody or trispecific binding fragment thereof treatment cycle, beginning on Day 1 of Cycle 2. In certain such embodiments, lenalidomide is administered daily for at least 21 days. In certain such embodiments, the BCMA×GPRC5D×CD3 trispecific antibody or trispecific binding fragment thereof treatment cycle is a 28-day cycle.

In some embodiments, lenalidomide is administered at a dose of about 25 mg. In certain such embodiments, lenalidomide is administered daily at a dose of about 25 mg.

In some embodiments, lenalidomide is administered for at least 21 days within the BCMA×GPRC5D×CD3 trispecific antibody or trispecific binding fragment thereof treatment cycle, wherein the administration begins on Day 1 of the cycle. In certain such embodiments, the BCMA×GPRC5D×CD3 trispecific antibody or trispecific binding fragment thereof treatment cycle is a 28-day cycle.

In some embodiments, lenalidomide is administered during at least two BCMA×GPRC5D×CD3 trispecific antibody or trispecific binding fragment thereof treatment cycles, wherein the administration begins at Day 1 of Cycle 2 at a dose of about 25 mg daily for at least 21 days and wherein the administration begins at Day 1 of any Cycle >2 at a dose of about 25 mg daily for at least 21 days. In certain such embodiments, each of the BCMA×GPRC5D×CD3 trispecific antibody or trispecific binding fragment thereof treatment cycles is a 28-day cycle. In some embodiments, lenalidomide is administered orally at a dose of about 25 mg daily for 21 days during a 28-day cycle, commencing on Cycle 2 Day 1. In some embodiments, the dose of lenalidomide is adjusted based on patient tolerance and hematologic toxicity.

In some embodiments, dexamethasone is administered during at least one BCMA×GPRC5D×CD3 trispecific antibody or trispecific binding fragment thereof treatment cycle. In certain such embodiments, dexamethasone is administered in conjunction with lenalidomide.

In some embodiments, dexamethasone is administered during at least one BCMA×GPRC5D×CD3 trispecific antibody or trispecific binding fragment thereof treatment cycle, beginning on Day 1 of Cycle 2. In certain such embodiments, dexamethasone is administered weekly, beginning on Day 1 of Cycle 2. In certain such embodiments, dexamethasone is administered once every week (weekly) for three BCMA×GPRC5D×CD3 trispecific antibody or trispecific binding fragment thereof treatment cycles, beginning on Day 1 of Cycle 2. In certain such embodiments, the dexamethasone is administered on Day 1, Day 8, Day 15, and Day 22 for three BCMA×GPRC5D×CD3 trispecific antibody or trispecific binding fragment thereof treatment cycles (Cycle 2, Cycle 3, and Cycle 4). In certain such embodiments, each of the BCMA×GPRC5D×CD3 trispecific antibody or trispecific binding fragment thereof treatment cycles is a 28-day cycle.

In some embodiments, dexamethasone is administered at a dose of about 20 mg. In some embodiments, dexamethasone is administered at a dose of about 40 mg. In certain such embodiments, dexamethasone is administered weekly at a dose of about 20 mg. In certain such embodiments, dexamethasone is administered weekly at a dose of about 40 mg.

In some embodiments, dexamethasone is administered in conjunction with lenalidomide. In some embodiments, dexamethasone is administered at a dose of about 20 mg orally on Days 1, 8, 15, and 22 of a 28-day cycle, commencing on Cycle 2 Day 1. In certain such embodiments, dexamethasone is administered at a dose of about 20 mg orally on Days 1, 8, 15, and 22 of a 28-day cycle for three 28-day cycles, commencing on Cycle 2 Day 1. In some embodiments, dexamethasone is administered at a dose of about 40 mg orally on Days 1, 8, 15, and 22 of a 28-day cycle, commencing on Cycle 2 Day 1. In certain such embodiments, dexamethasone is administered at a dose of about 40 mg orally on Days 1, 8, 15, and 22 of a 28-day cycle for three 28-day cycles, commencing on Cycle 2 Day 1.

In some embodiments, the treatment dose of the anti-CD38 antibody is administered during at least one BCMA×GPRC5D×CD3 trispecific antibody or trispecific binding fragment thereof treatment cycle and lenalidomide is administered during at least one BCMA×GPRC5D×CD3 trispecific antibody or trispecific binding fragment thereof treatment cycle. In certain such embodiments, the treatment dose of the anti-CD38 antibody is administered during at least one BCMA×GPRC5D×CD3 trispecific antibody or trispecific binding fragment thereof treatment cycle, beginning on Day 1 of Cycle 1, and lenalidomide is administered during at least one BCMA×GPRC5D×CD3 trispecific antibody or trispecific binding fragment thereof treatment cycle, beginning on Day 1 of Cycle 2.

In some embodiments, the treatment dose of the anti-CD38 antibody is administered during each BCMA×GPRC5D×CD3 trispecific antibody or trispecific binding fragment thereof treatment cycle, beginning on Day 1 of Cycle 1, and lenalidomide is administered during each BCMA×GPRC5D×CD3 trispecific antibody or trispecific binding fragment thereof treatment cycle, beginning on Day 1 of Cycle 2. In certain such embodiments, the treatment dose of the anti-CD38 antibody is administered once every four weeks for each BCMA×GPRC5D×CD3 trispecific antibody or trispecific binding fragment thereof treatment cycle. In certain such embodiments, lenalidomide is administered daily for at least 21 days. In certain such embodiments, the BCMA×GPRC5D×CD3 trispecific antibody or trispecific binding fragment thereof treatment cycle is a 28-day cycle.

In some embodiments, the treatment dose of the anti-CD38 antibody is administered during each BCMA×GPRC5D×CD3 trispecific antibody or trispecific binding fragment thereof treatment cycle, beginning on Day 1 of Cycle 1, and lenalidomide is administered during each BCMA×GPRC5D×CD3 trispecific antibody or trispecific binding fragment thereof treatment cycle, beginning on Day 1 of Cycle 2. In certain such embodiments, the treatment dose of the anti-CD38 antibody is administered once every week (weekly) for two BCMA×GPRC5D×CD3 trispecific antibody or trispecific binding fragment thereof treatment cycles (Cycle 1 and Cycle 2), followed by administration once every two weeks (biweekly) for four BCMA×GPRC5D×CD3 trispecific antibody or trispecific binding fragment thereof treatment cycles (Cycle 3, Cycle 4, Cycle 5 and Cycle 6), followed by administration once every four weeks for subsequent BCMA×GPRC5D×CD3 trispecific antibody or trispecific binding fragment thereof treatment cycles (≥Cycle 7). In certain such embodiments, the treatment dose of the anti-CD38 antibody is administered on Day 1, Day 8, Day 15, and Day 22 for two BCMA×GPRC5D×CD3 trispecific antibody or trispecific binding fragment thereof treatment cycles (Cycle 1 and Cycle 2); on Day 1 and Day 15 for four BCMA×GPRC5D×CD3 trispecific antibody or trispecific binding fragment thereof treatment cycles (Cycle 3, Cycle 4, Cycle 5 and Cycle 6); and on Day 1 for subsequent BCMA×GPRC5D×CD3 trispecific antibody or trispecific binding fragment thereof treatment cycles (≥Cycle 7). In certain such embodiments, lenalidomide is administered daily for at least 21 days. In certain such embodiments, the BCMA×GPRC5D×CD3 trispecific antibody or trispecific binding fragment thereof treatment cycle is a 28-day cycle.

In some embodiments, the treatment dose of the anti-CD38 antibody is 1800 mg, and lenalidomide is administered at a dose of about 25 mg. In some embodiments, the anti-CD38 antibody is daratumumab. In certain such embodiments, the daratumumab is formulated in a pharmaceutical composition comprising 1800 mg daratumumab and 30000 units hyaluronidase.

In some embodiments, dexamethasone is administered in conjunction with lenalidomide. In certain such embodiments, dexamethasone is administered during at least one BCMA×GPRC5D×CD3 trispecific antibody or trispecific binding fragment thereof treatment cycle, beginning on Day 1 of Cycle 2. In certain such embodiments, dexamethasone is administered weekly, beginning on Day 1 of Cycle 2. In certain such embodiments, dexamethasone is administered once every week (weekly) for three BCMA×GPRC5D×CD3 trispecific antibody or trispecific binding fragment thereof treatment cycles, beginning on Day 1 of Cycle 2. In certain such embodiments, the dexamethasone is administered on Day 1, Day 8, Day 15, and Day 22 for three BCMA×GPRC5D×CD3 trispecific antibody or trispecific binding fragment thereof treatment cycles (Cycle 2, Cycle 3, and Cycle 4). In certain such embodiments, each of the BCMA×GPRC5D×CD3 trispecific antibody or trispecific binding fragment thereof treatment cycles is a 28-day cycle.

In some embodiments, dexamethasone is administered at a dose of about 20 mg. In some embodiments, dexamethasone is administered at a dose of about 40 mg. In certain such embodiments, dexamethasone is administered weekly at a dose of about 20 mg. In certain such embodiments, dexamethasone is administered weekly at a dose of about 40 mg.

### Step-Up Dosing

In some embodiments, at least one step-up dose of the BCMA×GPRC5D×CD3 trispecific antibody or trispecific binding fragment thereof is administered prior to the administration of the treatment dose of the BCMA×GPRC5D×CD3 trispecific antibody or trispecific binding fragment thereof. In some embodiments, one step-up dose of the BCMA×GPRC5D×CD3 trispecific antibody or trispecific binding fragment thereof is administered.

Step-up doses are lower than the treatment dose. To prevent or lessen certain toxicities, such as cytokine release syndrome (CRS), a “priming” dose strategy may include one or more lower step-up dose(s) followed by higher treatment doses.

In some embodiments, the step-up dose(s) of the BCMA×GPRC5D×CD3 trispecific antibody or trispecific binding fragment thereof is administered intravenously, intramuscularly, intraperitoneally, and/or subcutaneously to the subject. In some embodiments, the step-up dose(s) is administered intravenously or subcutaneously to the subject. In some embodiments, the step-up dose(s) is administered intravenously to the subject. In one embodiment, the step-up dose(s) is administered subcutaneously to the subject. In some embodiments, the step-up dose(s) is administered using a different administration route than the treatment dose. In another embodiment, the step-up dose(s) is administered using the same administration route as the treatment dose.

In an embodiment, at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof. In some embodiments, the first step-up dose is about 2.5 mg to about 5 mg.

In certain embodiments, the first step-up dose is about 2.5 mg In another embodiment, the first step-up dose is about 5 mg.

In some embodiments, the first step-up dose is administered about 2-8 days prior to the administration of the treatment dose. In some embodiments, the first step-up dose is administered about one week prior to the administration of the treatment dose. In some embodiments, the first step-up dose is administered about 6-8 days prior to the administration of the treatment dose. In some embodiments, the first step-up dose is administered about 1-5 days prior to the administration of the treatment dose. In some embodiments, the first step-up dose is administered about 2-4 days prior to the administration of the treatment dose.

In some embodiments, the first step-up dose is subcutaneously administered about 2-8 days prior to the subcutaneous administration of the treatment dose. In some embodiments, the first step-up dose is subcutaneously administered about one week prior to the subcutaneous administration of the treatment dose. In some embodiments, the first step-up dose is subcutaneously administered about 6-8 days prior to the subcutaneous administration of the treatment dose. In some embodiments, the first step-up dose is subcutaneously administered about 1-5 days (e.g. about 2-4 days) prior to the subcutaneous administration of the treatment dose. In some embodiments, the first step-up dose is subcutaneously administered about 2-8 days prior to the subcutaneous administration of the treatment dose, and wherein the first step-up dose is about 5 mg. In one embodiment, the first step-up dose is subcutaneously administered about one week prior to the subcutaneous administration of the treatment dose, and wherein the first step-up dose is about 5 mg. In another embodiment, the first step-up dose is subcutaneously administered about 6-8 days prior to the subcutaneous administration of the treatment dose, and wherein the first step-up dose is about 5 mg. In yet another embodiment, the first step-up dose is subcutaneously administered about 2-4 days prior to the subcutaneous administration of the treatment dose, and wherein the first step-up dose is about 2.5 mg. In a further embodiment, the first step-up dose is subcutaneously administered about 2-4 days prior to the subcutaneous administration of the treatment dose, and wherein the first step-up dose is about 5 mg.

In some embodiments, the first step-up dose is a flat dose. For example, in some embodiments, the first step-up dose is a flat dose of about 2.5 mg to about 50 mg. In certain such embodiments, the first step-up dose is a flat dose of about 2.5 mg to about 5 mg.

In some embodiments, the first step-up dose is a flat dose of no greater than about 5 mg, or no greater than about 2.5 mg.

In certain embodiments, the first step-up dose is a flat dose of about 2.5 mg, or about 5 mg. In one embodiment, the first step-up dose is a flat dose of about 2.5 mg. In another embodiment, the first step-up dose is a flat dose of about 5 mg.

In some embodiments, the first step-up dose is no greater than one tenth of the treatment dose. In some embodiments, the first step-up dose is no greater than one twentieth of the treatment dose. In some embodiments, the first step-up dose is no greater than one twenty-fifth of the treatment dose. In some embodiments, the first step-up dose is no greater than one fortieth of the treatment dose. In some embodiments, the second step-up dose is no greater than one half of the treatment dose. In some embodiments, the second step-up dose is no greater than one third of the treatment dose. In some embodiments, the second step-up dose is no greater than one fifth of the treatment dose. In some embodiments, the second step-up dose is no greater than one seventh of the treatment dose. In some embodiments, the first step-up dose is no greater than one tenth of the treatment dose and the second step-up dose is no greater than one half of the treatment dose. In some embodiments, the first step-up dose is no greater than one twentieth of the treatment dose and the second step-up dose is no greater than one third of the treatment dose. In some embodiments, the first step-up dose is no greater than one twenty-fifth of the treatment dose and the second step-up dose is no greater than one fifth of the treatment dose. In some embodiments, the first step-up dose is no greater than one fortieth of the treatment dose and the second step-up dose is no greater than one seventh of the treatment dose.

In some embodiments, no step-up doses are administered prior to the administration of the treatment dose.

### Representative Dosing Regiments and Combination Therapies

In certain embodiments, representative dosing regimens, patient populations, and co-administration schedules for the BCMA×GPRC5D×CD3 trispecific antibody (BGCB491) are summarized in Table 1. The regimens described herein illustrate exemplary clinical settings and dosing configurations encompassed by the present disclosure and are not intended to limit the scope of the invention. Each regimen differs primarily in patient treatment history, the presence or absence of immunomodulatory agents, and the frequency of trispecific antibody administration across treatment cycles.

Regimen A represents administration of BGCB491 in combination with daratumumab on a once-every-four-week (Q4W) schedule, suitable for subjects having received one or more prior lines of therapy, including newly diagnosed or relapsed/refractory multiple myeloma (MM) with or without high-risk cytogenetic features.

Regimen C represents an adaptive schedule for newly diagnosed MM with high-risk cytogenetics, in which BGCB491 and daratumumab are administered subcutaneously according to a step-down frequency—weekly during Cycles 1-2, biweekly during Cycles 3-6, and monthly beginning at Cycle 7—to accommodate response and tolerability considerations.

Regimen D represents a triplet combination comprising BGCB491, daratumumab, and lenalidomide, in which dexamethasone is co-administered during Cycles 2-4. Dexamethasone is typically administered orally at a dose of about 40 mg once weekly (Days 1, 8, 15, 22) and may be reduced to about 20 mg weekly in elderly or low-body-mass subjects. Lenalidomide is self-administered orally at about 10 mg daily on Days 1-21 of each 28-day cycle beginning at Cycle 2.

Regimen E represents an extended immunomodulatory schedule wherein lenalidomide administration continues beyond Cycle 4, and dexamethasone may be maintained at investigator discretion to enhance IMID-driven anti-myeloma activity.

Across all regimens, BGCB491 is administered subcutaneously with a step-up dose of about 5 mg followed by a treatment dose of about 50-100 mg depending on cohort. Daratumumab is administered subcutaneously at a fixed dose of 1800 mg daratumumab/30 000 units hyaluronidase, typically prior to BGCB491, with an interval of approximately 3 hours±30 minutes before the trispecific administration. Dexamethasone and lenalidomide dosing schedules, when present, are coordinated with Cycles 2-4 or beyond as indicated. These regimens exemplify dosing and sequencing patterns encompassed by the therapeutic methods of the present invention.

A summary of representative treatment regimens, corresponding patient populations, and dosing parameters is provided in Table 1 below, followed by detailed administration tables (Tables 7-10 in the Examples) describing specific cycle structures and procedural guidance.

TABLE 1
Summary of Representative Treatment Regimens,
Patient Populations, and Dosing Parameters

| Prior | | | | |
|---|---|---|---|---|
| treatment | BGCB491 | Daratumumab) | Lenaalidomide | Dexamethasone |
| 1-3 prior | Day −8 to Day −2 | Daratumumab | | |
| lines/ | 5 mg step up prior to | SC 1800 mg | | |
| NDMM or | treatment dose | Q4W | | |
| RRMM | Treatment dose: | | | |
| Regime | 100 mg | | | |
| A1/A2 | Q4W | | | |
| NDMM | Day −8 to Day −2 | Daratumumab | | |
| (as of | 5 mg step up prior to | SC 1800 mg | | |
| August 2025 | treatment dose | Cycles (28-day | | |
| w/high-risk | Treatment dose: | cycle): | | |
| cytogenetics | 100 mg | Cycles 1 and 2: | | |
| Regime C | Q4W | Days 1, 8, 15 and | | |
| | | 22 (weekly) | | |
| | | Cycles 3 to 6: | | |
| | | Days 1 and 15 | | |
| | | (biweekly) | | |
| | | Cycles 7+: Day 1 | | |
| NDMM | Day −8 to Day −2 | Daratumumab | 25 mg PO | Dexamethasone |
| Regime D | 5 mg step up prior to | SC 1800 mg | Cycle 2+: Days | will be given |
| | treatment dose | Q4W | 1 to 21 | concomitantly |
| | Treatment dose: | | | with |
| | 100 mg | | | lenalidomide for |
| | Q4W | | | Cycles 2 to 4. |
| | | | | The dose is |
| | | | | dexamethasone |
| | | | | 40 mg weekly |
| | | | | (Days 1, 8, 15, |
| | | | | 22) from Cycle |
| | | | | 2 Day 1 through |
| | | | | the end of Cycle |
| | | | | 4. For |
| | | | | participants >75 |
| | | | | years of age or ≤75 |
| | | | | years of age |
| | | | | and BMI <18.5, |
| | | | | dexamethasone |
| | | | | may be |
| | | | | administered at |
| | | | | a dose of 20 mg |
| | | | | weekly. |
| NDMM | Day −8 to Day −2 | Daratumumab | 25 mg PO | Dexamethasone |
| Regime E | 5 mg step up prior to | SC 1800 mg | Cycle 2+: Days | will be given |
| | treatment dose | Cycles (28-day | 1 to 21 | concomitantly with |
| | Treatment dose: | cycle): | | lenalidomide for |
| | 100 mg | Cycles 1 and 2: | | Cycles 2 to 4. |
| | Q4W | Days 1, 8, 15 and | | The dose is |
| | | 22 (weekly) | | dexamethasone |
| | | Cycles 3 to 6: | | 40 mg weekly |
| | | Days 1 and 15 | | (Days 1, 8, 15, |
| | | (biweekly) | | 22) from Cycle |
| | | Cycles 7+: Day 1 | | 2 Day 1 through |
| | | | | the end of Cycle |
| | | | | 4. For |
| | | | | participants >75 |
| | | | | years of age or ≤75 |
| | | | | years of age |
| | | | | and BMI <18.5, |
| | | | | dexamethasone |
| | | | | may be |
| | | | | administered at |
| | | | | a dose of 20 mg |
| | | | | weekl |

Notes:
BGCB491 step-up is 5 mg SC given 2-8 days before the first treatment dose in all regimens shown.
When only daratumumab + BGCB491 are given that day (Regimens A/C days without Len), daratumumab is administered first, followed by BGCB491 with the same 3 h (first dose)/1 h (subsequent doses) spacing.
All SC components are given at alternating abdominal sites; site/interval adjustments and delays are mandated if CRS/ICANS from the prior dose have not resolved.

### Dosing Regimens

### 20 mg Treatment Dose [BCMA×GPRC5D×CD3 Trispecific Antibody or Trispecific Binding Fragment Thereof]

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the daratumumab is formulated in a pharmaceutical composition comprising 1800 mg daratumumab and 30000 units hyaluronidase and wherein the subject is a human subject. Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 20 mg; and (b) subcutaneous administration of daratumumab once every four weeks, wherein the daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 20 mg; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the anti-CD38 antibody is daratumumab and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491; and (b) subcutaneous administration of daratumumab once every four weeks, wherein the daratumumab is formulated in a pharmaceutical composition comprising 1800 mg daratumumab and 30000 units hyaluronidase and wherein the subject is a human subject.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 20 mg, wherein the BCMA×GPRC5D×CD3 trispecific antibody or trispecific binding fragment thereof is BGCB491; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle.

In one embodiment, the treatment dose of the BCMA×GPRC5D×CD3 trispecific antibody or trispecific binding fragment thereof is about 20 mg and wherein the treatment dose is subcutaneously administered once every four weeks. In some embodiments, the BCMA×GPRC5D×CD3 trispecific antibody or trispecific binding fragment thereof is subcutaneously administered once every four weeks at a treatment dose of about 20 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-8 days prior to the administration of the treatment dose. In another embodiment, the BCMA×GPRC5D×CD3 trispecific antibody or trispecific binding fragment thereof is subcutaneously administered once every four weeks at a treatment dose of about 20 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-4 days prior to the administration of the treatment dose. In yet another embodiment, the BCMA×GPRC5D×CD3 trispecific antibody or trispecific binding fragment thereof is subcutaneously administered once every four weeks at a treatment dose of about 20 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 2.5 mg and is administered about 2-4 days prior to the administration of the treatment dose. In a further embodiment, the BCMA×GPRC5D×CD3 trispecific antibody or trispecific binding fragment thereof is subcutaneously administered once every four weeks at a treatment dose of about 20 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about one week prior to the administration of the treatment dose. In one embodiment, the BCMA×GPRC5D×CD3 trispecific antibody or trispecific binding fragment thereof is subcutaneously administered once every four weeks at a treatment dose of about 20 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 6-8 days prior to the administration of the treatment dose. Often such dosing regimens comprise further step-up doses prior to administration of the treatment dose.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 20 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 20 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 20 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 20 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

### 50 mg Treatment Dose [BCMA×GPRC5D×CD3 Trispecific Antibody or Trispecific Binding Fragment Thereof]

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises:

(a) subcutaneous administration of the BCMA×GPRC5D×CD3 trispecific antibody or trispecific binding fragment thereof once every four weeks at a treatment dose of about 50 mg; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 50 mg; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 50 mg, wherein the BCMA×GPRC5D×CD3 trispecific antibody or trispecific binding fragment thereof is BGCB491; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

In one embodiment, the treatment dose of the BCMA×GPRC5D×CD3 trispecific antibody or trispecific binding fragment thereof is about 50 mg and wherein the treatment dose is subcutaneously administered once every four weeks. In some embodiments, the BCMA×GPRC5D×CD3 trispecific antibody or trispecific binding fragment thereof is subcutaneously administered once every four weeks at a treatment dose of about 50 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-8 days prior to the administration of the treatment dose. In another embodiment, the BCMA×GPRC5D×CD3 trispecific antibody or trispecific binding fragment thereof is subcutaneously administered once every four weeks at a treatment dose of about 50 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-4 days prior to the administration of the treatment dose. In yet another embodiment, the BCMA×GPRC5D×CD3 trispecific antibody or trispecific binding fragment thereof is subcutaneously administered once every four weeks at a treatment dose of about 50 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 2.5 mg and is administered about 2-4 days prior to the administration of the treatment dose. In a further embodiment, the BCMA×GPRC5D×CD3 trispecific antibody or trispecific binding fragment thereof is subcutaneously administered once every four weeks at a treatment dose of about 50 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about one week prior to the administration of the treatment dose. In one embodiment, the BCMA×GPRC5D×CD3 trispecific antibody or trispecific binding fragment thereof is subcutaneously administered once every four weeks at a treatment dose of about 50 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 6-8 days prior to the administration of the treatment dose. Often such dosing regimens comprise further step-up doses prior to administration of the treatment dose.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 50 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 50 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 50 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 50 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

### 100 mg Treatment Dose [BCMA×GPRC5D×CD3 Trispecific Antibody or Trispecific Binding Fragment Thereof]

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 100 mg; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 100 mg; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 100 mg, wherein the BCMA×GPRC5D×CD3 trispecific antibody or trispecific binding fragment thereof is BGCB491; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

In one embodiment, the treatment dose of the BCMA×GPRC5D×CD3 trispecific antibody or trispecific binding fragment thereof is about 100 mg and wherein the treatment dose is subcutaneously administered once every four weeks. In some embodiments, the BCMA×GPRC5D×CD3 trispecific antibody or trispecific binding fragment thereof is subcutaneously administered once every four weeks at a treatment dose of about 100 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-8 days prior to the administration of the treatment dose. In another embodiment, the BCMA×GPRC5D×CD3 trispecific antibody or trispecific binding fragment thereof is subcutaneously administered once every four weeks at a treatment dose of about 100 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-4 days prior to the administration of the treatment dose. In yet another embodiment, the BCMA×GPRC5D×CD3 trispecific antibody or trispecific binding fragment thereof is subcutaneously administered once every four weeks at a treatment dose of about 100 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 2.5 mg and is administered about 2-4 days prior to the administration of the treatment dose. In a further embodiment, the BCMA×GPRC5D×CD3 trispecific antibody or trispecific binding fragment thereof is subcutaneously administered once every four weeks at a treatment dose of about 100 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about one week prior to the administration of the treatment dose. In one embodiment, the BCMA×GPRC5D×CD3 trispecific antibody or trispecific binding fragment thereof is subcutaneously administered once every four weeks at a treatment dose of about 100 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 6-8 days prior to the administration of the treatment dose. Often such dosing regimens comprise further step-up doses prior to administration of the treatment dose.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 100 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 100 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 100 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 100 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

### 200 mg Treatment Dose [BCMA×GPRC5D×CD3 Trispecific Antibody or Trispecific Binding Fragment Thereof]

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 200 mg; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 200 mg; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 200 mg, wherein the BCMA×GPRC5D×CD3 trispecific antibody or trispecific binding fragment thereof is BGCB491; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

In one embodiment, the treatment dose of the BCMA×GPRC5D×CD3 trispecific antibody or trispecific binding fragment thereof is about 200 mg and wherein the treatment dose is subcutaneously administered once every four weeks. In some embodiments, the BCMA×GPRC5D×CD3 trispecific antibody or trispecific binding fragment thereof is subcutaneously administered once every four weeks at a treatment dose of about 200 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-8 days prior to the administration of the treatment dose. In another embodiment, the BCMA×GPRC5D×CD3 trispecific antibody or trispecific binding fragment thereof is subcutaneously administered once every four weeks at a treatment dose of about 200 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-4 days prior to the administration of the treatment dose. In yet another embodiment, the BCMA×GPRC5D×CD3 trispecific antibody or trispecific binding fragment thereof is subcutaneously administered once every four weeks at a treatment dose of about 200 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 2.5 mg and is administered about 2-4 days prior to the administration of the treatment dose. In a further embodiment, the BCMA×GPRC5D×CD3 trispecific antibody or trispecific binding fragment thereof is subcutaneously administered once every four weeks at a treatment dose of about 200 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about one week prior to the administration of the treatment dose. In one embodiment, the BCMA×GPRC5D×CD3 trispecific antibody or trispecific binding fragment thereof is subcutaneously administered once every four weeks at a treatment dose of about 200 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 6-8 days prior to the administration of the treatment dose. Often such dosing regimens comprise further step-up doses prior to administration of the treatment dose.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 200 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 200 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 200 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 200 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

### 300 mg Treatment Dose [BCMA×GPRC5D×CD3 Trispecific Antibody or Trispecific Binding Fragment Thereof]

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 300 mg; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 300 mg; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and an anti-CD38 antibody to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the anti-CD38 antibody is daratumumab and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the daratumumab is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratamumab to the subject to treat the multiple myeloma, wherein the method comprises subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491, wherein the daratumumab is formulated in a pharmaceutical composition comprising 1800 mg daratumumab and 30000 units hyaluronidase and is administered subcutaneously and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491; and (b) subcutaneous administration of daratumumab once every four weeks, wherein the daratumumab is formulated in a pharmaceutical composition comprising 1800 mg daratumumab and 30000 units hyaluronidase and wherein the subject is a human subject. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

Provided herein is a method of treating multiple myeloma in a subject in need thereof, comprising administering a therapeutically effective amount of a BCMA×GPRC5D×CD3 trispecific antibody or trispecific binding fragment thereof, and daratumumab to the subject to treat the multiple myeloma, wherein the method comprises: (a) subcutaneous administration of the BCMA×GPRC5D×CD3 trispecific antibody or trispecific binding fragment thereof once every four weeks at a treatment dose of about 300 mg, wherein the BCMA×GPRC5D×CD3 trispecific antibody or trispecific binding fragment thereof is BGCB491; and (b) subcutaneous administration of daratumumab once every week (weekly) for two treatment cycles, followed by subcutaneous administration of daratumumab once every two weeks (biweekly) for four treatment cycles, followed by subcutaneous administration of daratumumab once every four weeks for subsequent treatment cycles, wherein the daratumumab is formulated in a pharmaceutical composition comprising 1800 mg daratumumab and 30000 units hyaluronidase and wherein the subject is a human subject. In certain such embodiments, each treatment cycle is a 28-day cycle. In some embodiments, lenalidomide is co-administered. In some embodiments, lenalidomide is co-administered orally at a dose of about 25 mg on days 1 to 21 of a 28-day cycle. In some embodiments, lenalidomide is co-administered, commencing on Cycle 2 Day 1. In some embodiments, the dose of lenalidomide is adjusted based on patient tolerance and hematologic toxicity. In some embodiments, lenalidomide is administered in conjunction with dexamethasone. In some embodiments, lenalidomide is administered in conjunction with dexamethasone on designated days of the treatment cycle. In some embodiments, dexamethasone is administered at a dose of about 20 mg orally on Days 1, 8, 15, and 22 of the 28-day cycle. In some embodiments, dexamethasone is administered at a dose of about 40 mg orally on Days 1, 8, 15, and 22 of the 28-day cycle. In certain such embodiments, dexamethasone is administered for three 28-day cycles, commencing on Cycle 2 Day 1.

In one embodiment, the treatment dose of the BCMA×GPRC5D×CD3 trispecific antibody or trispecific binding fragment thereof is about 300 mg and wherein the treatment dose is subcutaneously administered once every four weeks. In some embodiments, the BCMA×GPRC5D×CD3 trispecific antibody or trispecific binding fragment thereof is subcutaneously administered once every four weeks at a treatment dose of about 300 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-8 days prior to the administration of the treatment dose. In another embodiment, the BCMA×GPRC5D×CD3 trispecific antibody or trispecific binding fragment thereof is subcutaneously administered once every four weeks at a treatment dose of about 300 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 2-4 days prior to the administration of the treatment dose. In yet another embodiment, the BCMA×GPRC5D×CD3 trispecific antibody or trispecific binding fragment thereof is subcutaneously administered once every four weeks at a treatment dose of about 300 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about one week prior to the administration of the treatment dose. In a further embodiment, the BCMA×GPRC5D×CD3 trispecific antibody or trispecific binding fragment thereof is subcutaneously administered once every four weeks at a treatment dose of about 300 mg, and wherein at least one step-up dose of the BCMA×GPRC5D×CD3 trispecific antibody or trispecific binding fragment thereof is subcutaneously administered prior to the subcutaneous administration of the treatment dose. In certain such embodiments, the first step-up dose is about 5 mg and is administered about 6-8 days prior to the administration of the treatment dose. Often such dosing regimens comprise further step-up doses prior to administration of the treatment dose.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 300 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 300 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about 2-8 days prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

In one embodiment, the BCMA×GPRC5D×CD3 trispecific antibody or trispecific binding fragment thereof and the anti-CD38 antibody are subcutaneously administered once every four weeks, wherein the treatment dose of the BCMA×GPRC5D×CD3 trispecific antibody or trispecific binding fragment thereof is about 300 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab and wherein the subject is a human subject.

In another embodiment, the BCMA×GPRC5D×CD3 trispecific antibody or trispecific binding fragment thereof is subcutaneously administered once every four weeks, wherein the treatment dose of the BCMA×GPRC5D×CD3 trispecific antibody or trispecific binding fragment thereof is about 300 mg, and wherein at least one step-up dose of the BCMA×GPRC5D×CD3 trispecific antibody or trispecific binding fragment thereof is subcutaneously administered prior to the subcutaneous administration of the treatment dose, wherein the step-up dose is about 5 mg and is administered about one week prior to the administration of the treatment dose, wherein the BCMA×GPRC5D×CD3 trispecific antibody or trispecific binding fragment thereof is BGCB491, wherein the anti-CD38 antibody is daratumumab, wherein daratumumab is subcutaneously administered once every week (weekly) for two treatment cycles (Cycle 1 and Cycle 2), once every two weeks (biweekly) for four treatment cycles (Cycle 3, Cycle 4, Cycle 5 and Cycle 6), and once every four weeks for subsequent treatment cycles (≥Cycle 7), and wherein the subject is a human subject. In certain such embodiments, each treatment cycle is a 28-day cycle.

Provided herein is a pharmaceutical composition comprising a BCMA×GPRC5D×CD3 trispecific antibody or a trispecific binding fragment thereof for use in treating multiple myeloma, wherein the composition is for administration, or to be administered, in combination with a pharmaceutical composition comprising an anti-CD38 antibody, and further wherein the BCMA×GPRC5D×CD3 trispecific antibody or trispecific binding fragment thereof is for subcutaneous administration at a treatment dose of at least about 20 mg. In one embodiment wherein the BCMA×GPRC5D×CD3 trispecific antibody or trispecific binding fragment thereof is for subcutaneous administration at a treatment dose of at least about 50 mg. In one embodiment wherein the BCMA×GPRC5D×CD3 trispecific antibody or trispecific binding fragment thereof is for subcutaneous administration at a treatment dose of at least about 100 mg. In one embodiment wherein the BCMA×GPRC5D×CD3 trispecific antibody or trispecific binding fragment thereof is for subcutaneous administration at a treatment dose of at least about 30 mg.

Provided herein is a pharmaceutical composition comprising an anti-CD38 antibody for use in treating multiple myeloma, wherein the composition is for administration, or to be administered, in combination with a pharmaceutical composition comprising a BCMA×GPRC5D×CD3 trispecific antibody or a trispecific binding fragment thereof, and further wherein the BCMA×GPRC5D×CD3 trispecific antibody or trispecific binding fragment thereof is for subcutaneous administration at a treatment dose of at least about 20 mg. In one embodiment wherein the BCMA×GPRC5D×CD3 trispecific antibody or trispecific binding fragment thereof is for subcutaneous administration at a treatment dose of at least about 50 mg. In one embodiment wherein the BCMA×GPRC5D×CD3 trispecific antibody or trispecific binding fragment thereof is for subcutaneous administration at a treatment dose of at least about 100 mg. In one embodiment wherein the BCMA×GPRC5D×CD3 trispecific antibody or trispecific binding fragment thereof is for subcutaneous administration at a treatment dose of at least about 30 mg.

In one embodiment, the treatment dose of the anti-CD38 is 1800 mg.

In some embodiments, the treatment dose of the anti-CD38 antibody is administered subcutaneously to the subject.

In one embodiment of the CD38 antibody is daratumumab, wherein daratumumab is formulated in a pharmaceutical composition comprising 1800 mg daratumumab and 30000 units hyaluronidase.

Dosage regimens in the above methods of treatment are adjusted to provide the optimum desired response (e.g., a therapeutic response). For example, the dose may be proportionally reduced or increased as indicated by the exigencies of the therapeutic situation. Parenteral compositions may be formulated in dosage unit form for ease of administration and uniformity of dosage.

The efficient dosages and the dosage regimens for the BCMA×GPRC5D×CD3 trispecific antibody or trispecific binding fragment thereof and for the anti-CD38 antibody, respectively, may be determined by one skilled in the art.

A physician, pharmacist or veterinarian having ordinary skill in the art may readily determine and prescribe the effective amount of the BCMA×GPRC5D×CD3 trispecific antibody or trispecific binding fragment thereof and for the anti-CD38 antibody, respectively, required. For example, the physician or veterinarian could start doses of the BCMA×GPRC5D×CD3 trispecific antibody or trispecific binding fragment thereof and for the anti-CD38 antibody, respectively, employed in the pharmaceutical composition at levels lower than that required in order to achieve the desired therapeutic effect and gradually increase the dosage until the desired effect is achieved. In general, a suitable daily dose of the BCMA×GPRC5D×CD3 trispecific antibody or trispecific binding fragment thereof and for the anti-CD38 antibody, respectively, of the disclosure will be that amount of the compound which is the lowest dose effective to produce a therapeutic effect.

The dosage may be determined or adjusted by measuring the amount of BCMA×GPRC5D×CD3 trispecific antibody or trispecific binding fragment thereof and for the anti-CD38 antibody, respectively, of the in the blood upon administration by for instance taking out a biological sample and using anti-idiotypic antibodies which target the GPRC5D and/or BCMA antigen binding arms of the BCMA×GPRC5D×CD3 trispecific antibody or trispecific binding fragment thereof and for the anti-CD38 antibody, respectively.

The doses depend on the desired effect, the duration of the treatment and the route of administration used. In general, the doctor will determine the appropriate dosage depending on the age, weight and any other factors specific to the subject to be treated.

The dosing regimens may be repeated one or more times as necessary, for example, after six months or twelve months.

In one embodiment, the trispecific antibody or trispecific binding fragment thereof may be administered by maintenance therapy, such as, e.g., once a week for a period of six months or more.

### Treatment of Multiple Myeloma (MM)

In some embodiments, the multiple myeloma is newly diagnosed multiple myeloma. In some embodiments, the multiple myeloma is relapsed, refractory, or malignant, or any combination thereof.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

The combination of a BCMA×GPRC5D×CD3 trispecific antibody or trispecific binding fragment thereof, and an anti-CD38 antibody may also be administered prophylactically in order to reduce the risk of developing multiple myeloma, delay the onset of the occurrence of an event in multiple myeloma progression, and/or reduce the risk of recurrence when a multiple myeloma is in remission. In some embodiments, lenalidomide is co-administered to further reduce the risk of developing multiple myeloma, further delay the onset of the occurrence of an event in multiple myeloma progression, and/or further reduce the risk of recurrence when a multiple myeloma is in remission.

In some embodiments, the multiple myeloma is a high-risk multiple myeloma. Subjects with high-risk multiple myeloma are known to relapse early and have poor prognosis and outcome. Subjects can be classified as having high-risk multiple myeloma if they have one or more of the following cytogenetic abnormalities: t(4; 14)(p16;q32), t(14; 16)(q32;q23), del17p, 1qAmp, t(4;14) (p16;q32) and t(14;16)(q32;q23), t(4;14) (p16;q32) and del17p, t(14;16)(q32;q23) and del17p, or t(4;14) (p16;q32), t(14;16)(q32;q23) and del17p. In some embodiments, the subject having the high-risk multiple myeloma has one or more chromosomal abnormalities comprising: t(4;14) (p16;q32), t(14;16)(q32;q23), del17p, 1qAmp, t(4;14) (p16;q32) and t(14;16)(q32;q23), t(4;14) (p16;q32) and del17p, t(14;16)(q32;q23) and del17p; or t(4;14) (p16;q32), t(14;16)(q32;q23) and del17p, or any combination thereof.

In some embodiments, the high-risk multiple myeloma is a newly diagnosed multiple myeloma. Newly diagnosed multiple myeloma subjects can be classified as having high-risk multiple myeloma if they exhibit one or more of the following cytogenetic abnormalities: del(17p), t(4;14), t(14;16), and 1q21 gain/1q21 amplification, or any combination thereof. In some embodiments, the newly diagnosed multiple myeloma subjects exhibit a del(17p), t(4;14), t(14;16) and 1q21 gain/1q21 amplification high-risk cytogenetic abnormality. In some embodiments, the newly diagnosed multiple myeloma subjects exhibit a del(17p), t(4;14), t(14;16) high-risk cytogenetic abnormality. In some embodiments, the newly diagnosed multiple myeloma subjects exhibit a del(17p) high-risk cytogenetic abnormality. In some embodiments, the newly diagnosed multiple myeloma subjects exhibit a del(17p), t(4;14) high-risk cytogenetic abnormality. In some embodiments, the newly diagnosed multiple myeloma subjects exhibit a del(17p), t(14;16) high-risk cytogenetic abnormality. In some embodiments, the newly diagnosed multiple myeloma subjects exhibit a del(17p), t(4;14), t(14;16) high-risk cytogenetic abnormality. In some embodiments, the newly diagnosed multiple myeloma subjects exhibit a del(17p), t(4;14) and 1q21 gain/1q21 amplification high-risk cytogenetic abnormality. In some embodiments, the newly diagnosed multiple myeloma subjects exhibit a del(17p), t(14;16) and 1q21 gain/1q21 amplification high-risk cytogenetic abnormality. In some embodiments, the newly diagnosed multiple myeloma subjects exhibit a 1q21 gain/1q21 amplification high-risk cytogenetic abnormality.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14) (p16;q32) involves translocation of fibroblast growth factor receptor 3 (FGFR3) and multiple myeloma SET domain containing protein (MMSET) (also called WHSC1/NSD2), and t(14; 16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

Particular trispecific antibodies that may be used to treat MM include BGCB491.

Particular anti-CD38 antibodies that may be used to treat MM include daratumumab.

In some embodiments, the BCMA×GPRC5D×CD3 trispecific antibody or trispecific binding fragment thereof and an anti-CD38 antibody is utilized for the treatment of relapsed or refractory multiple myeloma. In some embodiments, the BCMA×GPRC5D×CD3 trispecific antibody or trispecific binding fragment thereof and an anti-CD38 antibody is utilized for the treatment of relapsed multiple myeloma. In some embodiments, the BCMA×GPRC5D×CD3 trispecific antibody or trispecific binding fragment thereof and an anti-CD38 antibody is utilized for the treatment of refractory multiple myeloma. In some embodiments, lenalidomide is given in conjunction with the BCMA×GPRC5D×CD3 trispecific antibody and an anti-CD38 antibody for enhanced response rates.

In some embodiments, the subject is relapsed or refractory to treatment with a prior anti-cancer treatment. In some embodiments, the prior anti-cancer treatment comprises administering at least one of a proteasome inhibitor and immunomodulatory drug, such as bortezomib, carfilzomib, lenalidomide, or pomalidomide. In some embodiments, the subject is relapsed or refractory to treatment with an anti-CD38 antibody, selinexor, lenalinomide, bortezomib, pomalidomide, carfilzomib, elotozumab, ixazomib, melphalan, thalidomide, melphalan flufenamide, venetoclax or any combination thereof. In some embodiments, the anti-CD38 antibody is daratumumab or isatuximab. In some embodiments, the anti-CD38 antibody is daratumumab. In some embodiments, the subject is refractory to treatment with a proteasome inhibitor (PI). In certain such embodiments, the PI is bortezomib, carfilzomib, or ixazomib. In some embodiments, the subject is refractory to treatment with an immunomodulatory agent (IMiD). In certain such embodiments, the IMiD is thalidomide, lenalidomide, or pomalidomide. In some embodiments, the subject is refractory to treatment with CAR-T. In some embodiments, the subject is refractory to treatment with a proteasome inhibitor (PI), an immunomodulatory agent, and an anti-CD38 antibody. In some embodiments, the subject is refractory to treatment with two proteasome inhibitors (PI), two immunomodulatory agents, and an anti-CD38 antibody. In some embodiments, the subject is refractory to treatment with a BCMA directed therapy. In some embodiments, the subject is refractory to treatment with a GPRC5D directed therapy. In some embodiments, the subject is refractory to treatment with the last line of prior therapy.

In some embodiments, the subject receiving the BCMA×GPRC5D×CD3 trispecific antibody or binding fragment thereof has received a prior treatment. In some embodiments, the prior treatment is a BCMA or GPRC directed therapy. In some embodiments, the prior BCMA or GPRC directed therapy is CAR-T therapy. In certain such embodiments, the CAR-T therapy is Carvykti® (ciltacabtagene autoleucel). In some embodiments, the prior BCMA or GPRC directed therapy is a bispecific antibody. In some embodiments, the prior treatment is a BCMA directed therapy. In some embodiments, the prior BCMA directed therapy is CAR-T therapy. In certain such embodiments, the CAR-T therapy is Carvykti® (ciltacabtagene autoleucel). In some embodiments, the prior BCMA directed therapy is a bispecific antibody. In some embodiments, the prior treatment is a GPRC directed therapy, such as a GPRC5D directed therapy. In some embodiments, the prior GPRC directed therapy is CAR-T therapy. In some embodiments, the prior GPRC directed therapy is a bispecific antibody. In other embodiments, the patient is naïve to prior BCMA or GPRC directed therapy. In some embodiments, the prior treatment comprises a proteasome inhibitor, an immunomodulatory drug, a CD38 antibody, a CAR-T therapy, or any combination thereof. For example, the subject may have received one or more therapeutics, such as proteasome inhibitors (PIs) (e.g., Marizomib (salinosporamide A), Carfilzomib, Ixazomib), immunomodulatory drugs (IMiDs), CAR-T therapies, and/or anti-CD38 antibodies, for treating multiple myeloma. In certain such embodiments, the PI is bortezomib, carfilzomib or ixazomib. In certain such embodiments, the IMiD is lenalidomide, pomalidomide or thalidomide. In certain such embodiments, the anti-CD38 antibody is daratumumab or isatuximab.

In certain such embodiments, the CAR-T therapy is a BCMA CAR-T therapy. In certain such embodiments, the BCMA CAR-T therapy is Carvykti® (ciltacabtagene autoleucel). In some embodiments, the subject receiving the BCMA×GPRC5D×CD3 trispecific antibody or binding fragment thereof has received two prior PIs, two prior IMiDs, and a prior anti-CD38 antibody. In some embodiments, the prior treatment is selinexor. In some embodiments, the prior treatment is venetoclax. In some embodiments, the prior treatment is an antibody-drug conjugate. In some embodiments, the subject has received under three lines of prior therapy. In some embodiments, the subject has received over three lines of prior therapy. In some embodiments, the subject has received 1-3 lines of prior therapy. In some embodiments, the subject suffers from relapsed or refractory multiple myeloma and has received prior treatment with a proteasome inhibitor, an immunomodulatory drug (IMiD), and an anti-CD38-based therapy.

In one embodiment the multiple myeloma is newly diagnosed multiple myeloma.

In one embodiment the multiple myeloma is relapsed or refractory multiple myeloma (RRMM).

In one embodiment the relapsed or refractory multiple myeloma is relapsed multiple myeloma.

In one embodiment n the relapsed or refractory multiple myeloma is refractory multiple myeloma.

In one embodiment the subject is relapsed or refractory to treatment with a prior anti-cancer treatment.

In one embodiment the prior anti-cancer treatment comprises administering at least one of a proteasome inhibitor and immunomodulatory drug, such as bortezomib, carfilzomib, lenalidomide, or pomalidomide.

In one embodiment the subject has received a prior treatment.

In one embodiment the prior treatment comprises a proteasome inhibitor, and/or an immunomodulatory drug, or any combination thereof.

In one embodiment the subject has received 1 line of prior treatment.

In one embodiment the subject has received >1 line of prior treatment.

In one embodiment the subject has received 2 lines of prior treatment.

In one embodiment the subject has received >2 lines of prior treatment.

In one embodiment the subject has received 3 lines of prior treatment.

In one embodiment the subject has received 1-3 lines of prior treatment.

In one embodiment the subject is relapsed or refractory to the lines of prior treatment

In one embodiment the subject is relapsed or refractory to the 1-3 lines of prior treatment.

.

In some embodiments, the subject suffers from relapsed or refractory multiple myeloma and has received 1-3 lines of prior therapy. In certain such embodiments, the subject has received a prior PI. In certain such embodiments, the subject has received prior lenalidomide. In certain such embodiments, the subject has received a prior PI and prior lenalidomide.

The disclosure provides that treatment with a BCMA×GPRC5D×CD3 trispecific antibody or trispecific binding fragment thereof and an anti-CD38 antibody, according to particular dosing regimens may lead to safe and effective outcomes for subjects in need of treatment for multiple myeloma (MM). In some embodiments, lenalidomide is given in conjunction with the BCMA×GPRC5D×CD3 trispecific antibody and an anti-CD38 antibody to further improve outcomes for subjects.

Thus, in some embodiments, the subject does not exhibit a dose-limiting toxicity (DLT) or Grade 5 treatment emergent adverse event (TEAE) following administration of the BCMA×GPRC5D×CD3 trispecific antibody or trispecific binding fragment thereof.

In some embodiments, the method achieves a partial response (PR), very good partial response (VGPR), complete response (CR) or stringent complete response (sCR) in the subject. In certain such embodiments, the method achieves a partial response (PR), very good partial response (VGPR), complete response (CR) or stringent complete response (sCR) in the subject, according to IMWG criteria.

In some embodiments, the method achieves a very good partial response (VGPR), complete response (CR) or stringent complete response (sCR) in the subject. In certain such embodiments, the method achieves a very good partial response (VGPR), complete response (CR) or stringent complete response (sCR) in the subject, according to IMWG criteria.

In some embodiments, the method achieves a complete response (CR) or stringent complete response (sCR) in the subject. In certain such embodiments, the method achieves a complete response (CR) or stringent complete response (sCR) in the subject, according to IMWG criteria.

In some embodiments, the method achieves a stringent complete response (sCR) in the subject. In certain such embodiments, the method achieves a stringent complete response (sCR) in the subject, according to IMWG criteria.

In some embodiments, the method achieves an overall response rate of about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of treated subjects. In one embodiment, the method achieves an overall response rate of about 50% of treated subjects. The overall response rate may be deciphered by calculating the proportion of patients who achieve a partial response, a very good partial response, a complete response, or a stringent complete response.

### Compositions

The BCMA×GPRC5D×CD3 trispecific antibody or trispecific binding fragment thereof can be formulated as a pharmaceutical composition. In some embodiments, the pharmaceutical compositions comprise: a) an effective amount of a trispecific antibody or trispecific binding fragment of the disclosure herein, and b) a pharmaceutically acceptable carrier, which may be inert or physiologically active.

The anti-CD38 antibody can be formulated as a pharmaceutical composition. In some embodiments, the pharmaceutical compositions comprise: a) an effective amount of an anti-CD38 antibody or an antigen-binding fragment of the disclosure herein, and b) a pharmaceutically acceptable carrier, which may be inert or physiologically active.

The lenalidomide can be formulated as a pharmaceutical composition. In some embodiments, the pharmaceutical compositions comprise: a) an effective amount of lenalidomide, and b) a pharmaceutically acceptable carrier, which may be inert or physiologically active. The formulation can further include excipients that enhance the solubility and bioavailability of lenalidomide, or that facilitate its administration in combination with the BCMA×GPRC5D×CD3 trispecific antibody and/or the anti-CD38 antibody.

As used herein, the term “pharmaceutically acceptable carriers” includes any and all solvents, dispersion media, coatings, antibacterial and antifungal agents, and the like that are physiologically compatible.

In some embodiments, the pharmaceutical composition further comprises one or more excipients. In some embodiments, the one or more excipients include, but are not limited to a buffering agent, a sugar, a surfactant, a chelator, or any combination thereof.

Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH.about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

The compositions provided herein may be in a variety of forms. These include for example liquid, semi-solid, and solid dosage forms, but the preferred form depends on the intended mode of administration and therapeutic application. Typical preferred compositions are in the form of injectable or infusible solutions.

Sterile compositions for parenteral administration can be prepared by incorporating the antibody, antibody fragment or antibody conjugate provided herein in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohol's, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The BCMA×GPRC5D×CD3 trispecific antibody or trispecific binding fragment thereof, the anti-CD38 antibody, and the lenalidomide provided herein are used for the treatment of multiple myeloma (MM). In some embodiments, the multiple myeloma is newly diagnosed multiple myeloma. In some embodiments, the multiple myeloma is a relapsed or refractory form of multiple myeloma. In some embodiments, the multiple myeloma is a relapsed form of multiple myeloma. In some embodiments, the multiple myeloma is a refractory form of multiple myeloma.

Accordingly, the pharmaceutical compositions provided herein are useful in the treatment or prevention of multiple myeloma. In some embodiments, the multiple myeloma is a relapsed or refractory form of multiple myeloma. In some embodiments, the multiple myeloma is newly diagnosed multiple myeloma.

A therapeutically effective amount of the BCMA×GPRC5D×CD3 trispecific antibody or trispecific binding fragment thereof and the anti-CD38 antibody, is administered to a subject in need thereof. In some embodiments, lenalidomide is coadministered with the BCMA×GPRC5D×CD3 trispecific antibody or trispecific binding fragment thereof and the anti-CD38 antibody. In some embodiments, the subject is a mammal, preferably a human.

### EMBODIMENTS

The disclosure provided herein also provides the following non-limiting embodiments.
1. A method of treating multiple myeloma in a subject in need thereof, comprising administering a therapeutically effective amount of a BCMA×GPRC5D×CD3 trispecific antibody or a trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of at least about 20 mg.
2. The method of embodiment 1, wherein the BCMA×GPRC5D×CD3 trispecific antibody or a trispecific binding fragment thereof and the anti-CD38 antibody treatment dose, respectively, is a flat dose.
3. The method of embodiment 1 or embodiment 2, wherein the BCMA×GPRC5D×CD3 trispecific antibody or a trispecific binding fragment thereof treatment dose is about 50 mg, about 100 mg, about 200 mg, or about 300 mg.
4. The method of embodiment 1, wherein the BCMA×GPRC5D×CD3 trispecific antibody or a trispecific binding fragment thereof treatment dose is about 100 mg, 200 mg, or 300 mg.
5. The method of embodiment 4, further comprising administering lenalidomide to the subject.
6. The method of embodiment 5, wherein lenalidomide is administered orally at a dose of about 25 mg daily for 21 days during a 28-day cycle, commencing on Cycle 2 Day 1.
7. The method of embodiment 5 or embodiment 6, wherein the dose of lenalidomide is adjusted based on patient tolerance and hematologic toxicity.
8. The method of any one of embodiments 5-7 further comprising administering dexamethasone in conjunction with lenalidomide.
9. The method of embodiment 8, wherein dexamethasone is administered at a dose of about 20 mg or 40 mg orally on Days 1, 8, 15, and 22 of the 28-day cycle.
10. The method of embodiment 9, wherein dexamethasone is administered for three 28-day cycles, commencing on Cycle 2 Day 1.
11. The method of any one of embodiments 1-10, wherein the anti-CD38 antibody is daratumumab.
12. The method of embodiment 11, wherein daratumumab is formulated in a pharmaceutical composition and administered subcutaneously.
13. The method of embodiments 11 or 12, wherein daratumumab is formulated in a pharmaceutical composition comprising 1800 mg daratumumab and 30000 units hyaluronidase.
14. The method of any one of embodiments 11-13, wherein daratumumab is formulated in a pharmaceutical composition comprising 1800 mg daratumumab and 30000 units hyaluronidase and is administered subcutaneously.
15. The method of any one of embodiments 1-14, wherein the BCMA×GPRC5D×CD3 trispecific antibody or a trispecific binding fragment thereof treatment dose is about 50 mg.
16. The method of any one of embodiments 1-14, wherein the BCMA×GPRC5D×CD3 trispecific antibody or a trispecific binding fragment thereof treatment dose is about 100 mg.
17. The method of any one of embodiments 1-14, wherein the BCMA×GPRC5D×CD3 trispecific antibody or a trispecific binding fragment thereof treatment dose is about 200 mg.
18. The method of any one of embodiments 1-14, wherein the BCMA×GPRC5D×CD3 trispecific antibody or a trispecific binding fragment thereof treatment dose is about 300 mg.
19. The method of any one of embodiments 1-18, wherein the treatment dose of the BCMA×GPRC5D×CD3 trispecific antibody or trispecific binding fragment thereof is administered once every four weeks.
20. The method of any one of embodiments 11-19, wherein the treatment dose of Daratumumab is administered once every four weeks.
21. The method of any one of embodiments 11-19, wherein the treatment dose of Daratumumab is administered once every week for two 28-day cycles, followed by administration once every two weeks for four 28-day cycles, followed by administration once every four weeks for subsequent 28-day cycles.
22. The method of any one of embodiments 1-21, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.
23. The method of embodiment 22, wherein the first step-up dose is about 5 mg.
24. The method of embodiment 22 or 23, wherein the first step-up dose is subcutaneously administered about 2-8 days prior to the subcutaneous administration of the treatment dose.
25. The method of embodiment 22 or 23, wherein the first step-up dose is subcutaneously administered about one week prior to the subcutaneous administration of the treatment dose.
26. The method of embodiment 22 or 23, wherein the first step-up dose is subcutaneously administered about 6-8 days prior to the subcutaneous administration of the treatment dose.
27. The method of embodiment 22 or 23, wherein the first step-up dose is subcutaneously administered about 2-4 days prior to the subcutaneous administration of the treatment dose.
28. The method of any one of embodiments 22 to 24, wherein the first step-up dose is subcutaneously administered about 2 to 8 days prior to the subcutaneous administration of the treatment dose, and wherein the first step-up dose is about 5 mg.
29. The method of any one of embodiments 22, 23 or 25, wherein the first step-up dose is subcutaneously administered about one week prior to the subcutaneous administration of the treatment dose, and wherein the first step-up dose is about 5 mg.
30. The method of any one of embodiments 22, 23 or 26, wherein the first step-up dose is subcutaneously administered about 6-8 days prior to the subcutaneous administration of the treatment dose, and wherein the first step-up dose is about 5 mg.
31. The method of any one of embodiments 22 or 27, wherein the first step-up dose is subcutaneously administered about 2-4 days prior to the subcutaneous administration of the treatment dose, and wherein the first step-up dose is about 2.5 mg.
32. The method of any one of embodiments 22, 23 or 27, wherein the first step-up dose is subcutaneously administered about 2-4 days prior to the subcutaneous administration of the treatment dose, and wherein the first step-up dose is about 5 mg.
33. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 50 mg and wherein the treatment dose is administered once every four weeks.
34. The method of embodiment 33, further comprising administering lenalidomide to the subject.
35. The method of embodiment 34, wherein lenalidomide is administered orally at a dose of about 25 mg daily for 21 days during a 28-day cycle, commencing on Cycle 2 Day 1.
36. The method of embodiment 34 or embodiment 35, wherein the dose of lenalidomide is adjusted based on patient tolerance and hematologic toxicity.
37. The method of any one of embodiments 34-36 further comprising administering dexamethasone in conjunction with lenalidomide.
38. The method of embodiment 37, wherein dexamethasone is administered at a dose of about 20 mg or 40 mg orally on Days 1, 8, 15, and 22 of the 28-day cycle.
39. The method of embodiment 38, wherein dexamethasone is administered for three 28-day cycles, commencing on Cycle 2 Day 1.
40. The method of any one of embodiments 33-39, wherein the anti-CD38 antibody is daratumumab.
41. The method of embodiment 40, wherein daratumumab is administered subcutaneously.
42. The method of embodiments 40 or 41, wherein daratumumab is formulated in a pharmaceutical composition comprising 1800 mg daratumumab and 30000 units hyaluronidase.
43. The method of any one of embodiments 40 to 42, wherein daratumumab is formulated in a pharmaceutical composition comprising 1800 mg daratumumab and 30000 units hyaluronidase and is administered subcutaneously.
44. The method of any one of embodiments 40 to 43, wherein daratumumab is administered once every four weeks.
45. The method of any one of embodiments 40 to 43, wherein daratumumab is administered once every week for two 28-day cycles, followed by administration once every two weeks for four 28-day cycles, followed by administration once every four weeks for subsequent 28-day cycles.
46. The method of any one of embodiments 33-45, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.
47. The method of embodiment 46, wherein the first step-up dose is about 2.5 mg and is administered about 2-4 days prior to administration of the treatment dose.
48. The method of embodiment 46, wherein the first step-up dose is about 5 mg and is administered about 2-4 days prior to administration of the treatment dose.
49. The method of embodiment 46, wherein the first step-up dose is about 5 mg and is administered about 2 to 8 days prior to administration of the treatment dose.
50. The method of embodiment 46, wherein the first step-up dose is about 5 mg and is administered about one week prior to administration of the treatment dose.
51. The method of embodiment 46, wherein the first step-up dose is about 5 mg and is administered about 6-8 days prior to administration of the treatment dose.
52. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 100 mg and wherein the treatment dose is administered once every four weeks.
53. The method of embodiment 52, further comprising administering lenalidomide to the subject.
54. The method of embodiment 53, wherein lenalidomide is administered orally at a dose of about 25 mg daily for 21 days during a 28-day cycle, commencing on Cycle 2 Day 1.
55. The method of embodiment 53 or embodiment 54, wherein the dose of lenalidomide is adjusted based on patient tolerance and hematologic toxicity.
56. The method of any one of embodiments 53-55 further comprising administering dexamethasone in conjunction with lenalidomide.
57. The method of embodiment 56, wherein dexamethasone is administered at a dose of about 20 mg or 40 mg orally on Days 1, 8, 15, and 22 of the 28-day cycle.
58. The method of embodiment 57, wherein dexamethasone is administered for three 28-day cycles, commencing on Cycle 2 Day 1.
59. The method of any one of embodiments 52-58, wherein the anti-CD38 antibody is daratumumab.
60. The method of embodiment 59, wherein daratumumab is administered subcutaneously.
61. The method of embodiments 59 or 60, wherein daratumumab is formulated in a pharmaceutical composition comprising 1800 mg daratumumab and 30000 units hyaluronidase.
62. The method of any one of embodiments 59 to 61, wherein daratumumab is formulated in a pharmaceutical composition comprising 1800 mg daratumumab and 30000 units hyaluronidase and is administered subcutaneously.
63. The method of any one of embodiments 59 to 62, wherein daratumumab is administered once every four weeks.
64. The method of any one of embodiments 59 to 62, wherein daratumumab is administered once every week for two 28-day cycles, followed by administration once every two weeks for four 28-day cycles, followed by administration once every four weeks for subsequent 28-day cycles.
65. The method of any one of embodiments 52-64, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.
66. The method of embodiment 65, wherein the first step-up dose is about 5 mg and is administered about 2-4 days prior to administration of the treatment dose.
67. The method of embodiment 65, wherein the first step-up dose is about 2.5 mg and is administered about 2-4 days prior to administration of the treatment dose.
68. The method of embodiment 65, wherein the first step-up dose is about 5 mg and is administered about 2-8 days prior to administration of the treatment dose.
69. The method of embodiment 65, wherein the first step-up dose is about 5 mg and is administered about one week prior to administration of the treatment dose.
70. The method of embodiment 65, wherein the first step-up dose is about 5 mg and is administered about 6-8 days prior to administration of the treatment dose.
71. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 200 mg and wherein the treatment dose is administered once every four weeks.
72. The method of embodiment 71, further comprising administering lenalidomide to the subject.
73. The method of embodiment 72, wherein lenalidomide is administered orally at a dose of about 25 mg daily for 21 days during a 28-day cycle, commencing on Cycle 2 Day 1.
74. The method of embodiment 72 or embodiment 73, wherein the dose of lenalidomide is adjusted based on patient tolerance and hematologic toxicity.
75. The method of any one of embodiments 72-74 further comprising administering dexamethasone in conjunction with lenalidomide.
76. The method of embodiment 75, wherein dexamethasone is administered at a dose of about 20 mg or 40 mg orally on Days 1, 8, 15, and 22 of the 28-day cycle.
77. The method of embodiment 76, wherein dexamethasone is administered for three 28-day cycles, commencing on Cycle 2 Day 1.
78. The method of any one of embodiments 71-77, wherein the anti-CD38 antibody is daratumumab.
79. The method of embodiment 78, wherein daratumumab is administered subcutaneously.
80. The method of embodiments 78 or 79, wherein daratumumab is formulated in a pharmaceutical composition comprising 1800 mg daratumumab and 30000 units hyaluronidase.
81. The method of any one of embodiments 78 to 80, wherein daratumumab is formulated in a pharmaceutical composition comprising 1800 mg daratumumab and 30000 units hyaluronidase and is administered subcutaneously.
82. The method of any one of embodiments 78 to 81, wherein daratumumab is administered once every four weeks.
83. The method of any one of embodiments 78 to 81, wherein daratumumab is administered once every week for two 28-day cycles, followed by administration once every two weeks for four 28-day cycles, followed by administration once every four weeks for subsequent 28-day cycles.
84. The method of any one of embodiments 71-83, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.
85. The method of embodiment 84, wherein the first step-up dose is about 5 mg and is administered about 2-4 days prior to administration of the treatment dose.
86. The method of embodiment 84, wherein the first step-up dose is about 2.5 mg and is administered about 2-4 days prior to administration of the treatment dose.
87. The method of embodiment 84, wherein the first step-up dose is about 5 mg and is administered about 2-8 days prior to administration of the treatment dose.
88. The method of embodiment 84, wherein the first step-up dose is about 5 mg and is administered about one week prior to administration of the treatment dose.
89. The method of embodiment 84, wherein the first step-up dose is about 5 mg and is administered about 6-8 days prior to administration of the treatment dose.
90. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 300 mg and wherein the treatment dose is administered once every four weeks.
91. The method of embodiment 90, further comprising administering lenalidomide to the subject.
92. The method of embodiment 91, wherein lenalidomide is administered orally at a dose of about 25 mg daily for 21 days during a 28-day cycle, commencing on Cycle 2 Day 1.
93. The method of embodiment 91 or embodiment 92, wherein the dose of lenalidomide is adjusted based on patient tolerance and hematologic toxicity.
94. The method of any one of embodiments 91-93 further comprising administering dexamethasone in conjunction with lenalidomide.
95. The method of embodiment 94, wherein dexamethasone is administered at a dose of about 20 mg or 40 mg orally on Days 1, 8, 15, and 22 of the 28-day cycle.
96. The method of embodiment 95, wherein dexamethasone is administered for three 28-day cycles, commencing on Cycle 2 Day 1.
97. The method of any one of embodiments 90-96, wherein the anti-CD38 antibody is daratumumab.
98. The method of embodiment 97, wherein daratumumab is administered subcutaneously.
99. The method of embodiments 97 or 98, wherein daratumumab is formulated in a pharmaceutical composition comprising 1800 mg daratumumab and 30000 units hyaluronidase.
100. The method of any one of embodiments 97 to 99, wherein daratumumab is formulated in a pharmaceutical composition comprising 1800 mg daratumumab and 30000 units hyaluronidase and is administered subcutaneously.
101. The method of any one of embodiments 97 to 100, wherein daratumumab is administered once every four weeks.
102. The method of any one of embodiments 97 to 100, wherein daratumumab is administered once every week for two 28-day cycles, followed by administration once every two weeks for four 28-day cycles, followed by administration once every four weeks for subsequent 28-day cycles.
103. The method of any one of embodiments 90-102, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.
104. The method of embodiment 103, wherein the first step-up dose is about 5 mg and is administered about 2-4 days prior to administration of the treatment dose.
105. The method of embodiment 103, wherein the first step-up dose is about 2.5 mg and is administered about 2-4 days prior to administration of the treatment dose.
106. The method of embodiment 103, wherein the first step-up dose is about 5 mg and is administered about 2-8 days prior to administration of the treatment dose.
107. The method of embodiment 103, wherein the first step-up dose is about 5 mg and is administered about one week prior to administration of the treatment dose.
108. The method of embodiment 103, wherein the first step-up dose is about 5 mg and is administered about 6-8 days prior to administration of the treatment dose.
109. The method of any one of embodiments 1-108, wherein the BCMA×GPRC5D×CD3 trispecific antibody or trispecific binding fragment thereof comprises:
   (a) a first antigen-binding arm comprising a first heavy chain variable domain (VH1) and a first light chain variable domain (VL1);
   (b) a second antigen-binding arm comprising a second heavy chain variable domain (VH2) and a second light chain variable domain (VL2);
   (c) a third antigen-binding arm comprising a third heavy chain variable domain (VH3) and a third light chain variable domain (VL3), wherein the first antigen-binding arm binds to an epitope on cluster of differentiation 3 (CD3), the second antigen-binding arm binds to an epitope on G-protein coupled receptor family C group 5 member D (GPRC5D), and the third antigen-binding arm binds to an epitope on B cell maturation antigen (BCMA).
110. The method of embodiment 109, wherein the VH1 and VL1 of first antigen-binding arm are present in a Fab, the VH2 and VL2 of the second antigen-binding arm are present in a scFv, and the VH3 and VL3 of the third antigen-binding arm are present in a scFv.
111. The method of embodiment 109 or embodiment 110, wherein the first antigen-binding arm that binds CD3 comprises a HCDR1 comprising the amino acid sequence of GDSVENNNAAWS (SEQ ID NO: 4), a HCDR2 comprising the amino acid sequence of RTYYRSKWLYD (SEQ ID NO: 5), and a HCDR3 comprising the amino acid sequence of GYSSSFDY (SEQ ID NO: 6); and a LCDR1 comprising the amino acid sequence of TGTSSNIGTYKFVS (SEQ ID NO: 1), a LCDR2 comprising the amino acid sequence of EVSKRPS (SEQ ID NO: 2), and a LCDR3 comprising the amino acid sequence of VSYAGSGTLL (SEQ ID NO: 3).
112. The method of any one of embodiments 109-111, wherein the first antigen-binding arm that binds CD3 comprises the VH1 of SEQ ID NO: 8 and the VL1 of SEQ ID NO: 7.
113. The method of any one of embodiments 109-112, wherein the second antigen-binding arm that binds GPRC5D comprises a HCDR1 comprising the amino acid sequence of GFSLTNIRMSVS (SEQ ID NO: 12), HCDR2 comprising the amino acid sequence of HIFSNDEKS (SEQ ID NO: 13), and a HCDR3 comprising the amino acid sequence of MRLPYGMDV (SEQ ID NO: 14); and a LCDR1 comprising the amino acid sequence of RSSQSLVHSDGNTYLS (SEQ ID NO: 9), a LCDR2 comprising the amino acid sequence of KISNRFF (SEQ ID NO: 10), and a LCDR3 comprising the amino acid sequence of MQATQFPHT (SEQ ID NO: 11).
114. The method of any one of embodiments 109-113, wherein the second antigen-binding arm that binds GPRC5D comprises the VH2 of SEQ ID NO: 16 and the VL2 of SEQ ID NO: 15.
115. The method of any one of embodiments 109-114, wherein the third antigen-binding arm that binds BCMA comprises a HCDR1 comprising the amino acid sequence of GFTFSSYAMS (SEQ ID NO: 20), a HCDR2 comprising the amino acid sequence of AISGSGGSTY (SEQ ID NO: 21), and a HCDR3 comprising the amino acid sequence of DEGYSSGHYYGMDV (SEQ ID NO: 22); and a LCDR1 comprising the amino acid sequence of RASQSISSSFLT (SEQ ID NO: 17), a LCDR2 comprising the amino acid sequence of GASSRAT (SEQ ID NO: 18), and a LCDR3 comprising the amino acid sequence of QHYGSSPMYT (SEQ ID NO: 19).
116. The method of any one of embodiments 109-115, wherein the third antigen-binding arm that binds BCMA comprises the VH3 of SEQ ID NO: 24 and the VL3 of SEQ ID NO: 23.
117. The method of any one of embodiments 109-116, wherein the first antigen-binding arm that binds CD3 comprises the HCDR1, the HCDR2, the HCDR3, the LCDR1, the LCDR2 and the LCDR3 of SEQ ID NOs: 4, 5, 6, 1, 2, 3, respectively; the second antigen-binding arm that binds GPRC5D comprises the HCDR1, the HCDR2, the HCDR3, the LCDR1, the LCDR2 and the LCDR3 of SEQ ID NOS: 12, 13, 14, 9, 10 and 11, respectively; and the third antigen-binding arm that binds BCMA comprises the HCDR1, the HCDR2, the HCDR3, the LCDR1, the LCDR2 and the LCDR3 of SEQ ID NOs: 20, 21, 22, 17, 18 and 19, respectively.
118. The method of any one of embodiments 109-117, wherein the first antigen-binding arm that binds CD3 comprises the VH1 of SEQ ID NO: 8 and the VL1 of SEQ ID NO: 7; the second antigen-binding arm that binds GPRC5D comprises the VH2 of SEQ ID NO: 16 and the VL2 of SEQ ID NO: 15; and the third antigen-binding arm that binds BCMA comprises the VH3 of SEQ ID NO: 24 and the VL3 of SEQ ID NO: 23.
119. The method of any one of embodiments 109-118, wherein the BCMA×GPRC5D×CD3 trispecific antibody comprises a first antigen-binding arm that binds to an epitope on cluster of differentiation 3 (CD3), a second antigen-binding arm that binds to an epitope on G-protein coupled receptor family C group 5 member D (GPRC5D), and a third antigen-binding arm that binds to an epitope on B cell maturation antigen (BCMA), wherein the first antigen-binding arm comprises a heavy chain (HC1) polypeptide and a light chain (LC) polypeptide, wherein the heavy chain (HC1) polypeptide further comprises the second antigen-binding arm, wherein the trispecific antibody, or the trispecific binding fragment thereof, further comprises a single polypeptide comprising the third antigen-binding arm.
120. The method of embodiment 119, wherein the HC1 of the first antigen-binding arm comprises the amino acid sequence of SEQ ID NO: 29.
121. The method of embodiment 119 or embodiment 120, wherein the LC of the first antigen-binding arm comprises the amino acid sequence of SEQ ID NO: 30.
122. The method of any one of embodiments 119-121, wherein the single polypeptide comprising the third antigen-binding arm comprises an amino acid sequence of SEQ ID NO: 31.
123. The method of any one of embodiments 119-122, wherein the first antigen-binding arm comprises an HC1 comprising the amino acid sequence of SEQ ID NO: 29, and a LC comprising the amino acid sequence of SEQ ID NO: 30, and the single polypeptide comprising the third antigen-binding arm comprises the amino acid sequence of SEQ ID NO: 31.
124. The method of any one of embodiments 109-123, wherein the BCMA×GPRC5D×CD3 trispecific antibody or trispecific binding fragment thereof is an (human) IgG1 isotype.
125. The method of any one of embodiments 1-124, wherein the multiple myeloma is newly diagnosed multiple myeloma.
126. The method of embodiment 125, wherein the subject exhibits high-risk cytogenetic abnormalities selected from the group consisting of del(17p), t(4;14), t(14;16), and 1q21 gain/1q21 amplification.
127. The method of embodiment 126, wherein the high-risk cytogenetic abnormalities are identified using fluorescence in situ hybridization (FISH) or next-generation sequencing (NGS).
128. The method of any one of embodiments 1-124, wherein the multiple myeloma is relapsed or refractory multiple myeloma (RRMM).
129. The method of embodiment 128, wherein the relapsed or refractory multiple myeloma is relapsed multiple myeloma.
130. The method of embodiment 128, wherein the relapsed or refractory multiple myeloma is refractory multiple myeloma.
131. The method of any one of embodiments 1-124 and 128-130 wherein the subject is relapsed or refractory to treatment with a prior anti-cancer treatment.
132. The method of embodiment 131, wherein the prior anti-cancer treatment comprises administering at least one of a proteasome inhibitor and immunomodulatory drug, such as bortezomib, carfilzomib, lenalidomide, or pomalidomide.
133. The method of any one of embodiments 1-124 and 128-132 wherein the subject has received a prior treatment.
134. The method of embodiment 133, wherein the prior treatment comprises a proteasome inhibitor, and/or an immunomodulatory drug, or any combination thereof.
135. The method of any one of embodiments 1-124 and 128-134, wherein the subject has received >1 lines of prior treatment.
136. The method of any one of embodiments 1-124 and 128-134, wherein the subject has received 1-3 lines of prior treatment.
137. The method of embodiment 135 or embodiment 136, wherein the subject is relapsed or refractory to the lines of prior treatment.
138. The method of any one of embodiments 1-137, wherein the subject is a human subject.
139. A pharmaceutical composition comprising a BCMA×GPRC5D×CD3 trispecific antibody or a trispecific binding fragment thereof for use in treating multiple myeloma, wherein the composition is for administration, or to be administered, in combination with a pharmaceutical composition comprising an anti-CD38 antibody, and further wherein the BCMA×GPRC5D×CD3 trispecific antibody or trispecific binding fragment thereof is for subcutaneous administration at a treatment dose of at least about 20 mg.
140. A pharmaceutical composition comprising an anti-CD38 antibody for use in treating multiple myeloma, wherein the composition is for administration, or to be administered, in combination with a pharmaceutical composition comprising a BCMA×GPRC5D×CD3 trispecific antibody or a trispecific binding fragment thereof, and further wherein the BCMA×GPRC5D×CD3 trispecific antibody or trispecific binding fragment thereof is for subcutaneous administration at a treatment dose of at least about 20 mg.
141. A pharmaceutical composition for use, as in embodiment 139 or embodiment 140, wherein the anti-CD38 antibody comprises a heavy chain complementarity determining region 1 (HCDR1) of SEQ ID NO: 201, a HCDR2 of SEQ ID NO: 202, a HCDR3 of SEQ ID NO: 203, a light chain complementarity determining region 1 (LCDR1) of SEQ ID NO: 204, a LCDR2 of SEQ ID NO: 205 and a LCDR3 of SEQ ID NO: 206.
142. A pharmaceutical composition for use, as in embodiment 141, wherein the anti-CD-38 antibody comprises a heavy chain variable region (VH) of SEQ ID NO: 207 and a light chain variable region (VL) of SEQ ID NO: 208.
143. A pharmaceutical composition for use, as in embodiment 141 or embodiment 142, wherein the anti-CD38 antibody is an IgG1 isotype.
144. A pharmaceutical composition for use, as in any one of embodiments 139 to 143, wherein the anti-CD38 antibody comprises a heavy chain (HC) of SEQ ID NO: 209 and a light chain (LC) of SEQ ID NO: 210.
145. A pharmaceutical composition for use, as in any one of embodiments 139 to 144, wherein the anti-CD-38 antibody is daratumumab.
146. A pharmaceutical composition for use, as in any one of embodiments 139-145, wherein the anti-CD38 antibody is formulated in a pharmaceutical formulation further comprising a hyaluronidase, optionally, wherein the hyaluronidase is a recombinant human hyaluronidase, optionally wherein the recombinant human hyaluronidase is rHuPH20.
147. A pharmaceutical composition for use as in any one of embodiments 139-146, wherein the pharmaceutical composition comprising an anti-CD38 antibody is administered subcutaneously.
148. A pharmaceutical composition for use as in any one of embodiments 139-147, wherein the pharmaceutical composition comprising an anti-CD38 antibody is formulated in a pharmaceutical composition comprising 1800 mg daratumumab and 30000 units hyaluronidase.
149. A pharmaceutical composition for use as in any one of embodiments 139 to 148, wherein the BCMA×GPRC5D×CD3 trispecific antibody or trispecific binding fragment thereof comprises:
   (a) a first antigen-binding arm comprising a first heavy chain variable domain (VH1) and a first light chain variable domain (VL1);
   (b) a second antigen-binding arm comprising a second heavy chain variable domain (VH2) and a second light chain variable domain (VL2);
   (c) a third antigen-binding arm comprising a third heavy chain variable domain (VH3) and a third light chain variable domain (VL3), wherein the first antigen-binding arm binds to an epitope on cluster of differentiation 3 (CD3), the second antigen-binding arm binds to an epitope on G-protein coupled receptor family C group 5 member D (GPRC5D), and the third antigen-binding arm binds to an epitope on B cell maturation antigen (BCMA).
150 The pharmaceutical composition for use as in embodiment 149, wherein the VH1 and VL1 of first antigen-binding arm are present in a Fab, the VH2 and VL2 of the second antigen-binding arm are present in a scFv, and the VH3 and VL3 of the third antigen-binding arm are present in a scFv.
151. The pharmaceutical composition for use as in embodiments 149 or 150, wherein the first antigen-binding arm that binds CD3 comprises a HCDR1 comprising the amino acid sequence of GDSVENNNAAWS (SEQ ID NO: 4), a HCDR2 comprising the amino acid sequence of RTYYRSKWLYD (SEQ ID NO: 5), and a HCDR3 comprising the amino acid sequence of GYSSSFDY (SEQ ID NO: 6); and a LCDR1 comprising the amino acid sequence of TGTSSNIGTYKFVS (SEQ ID NO: 1), a LCDR2 comprising the amino acid sequence of EVSKRPS (SEQ ID NO: 2), and a LCDR3 comprising the amino acid sequence of VSYAGSGTLL (SEQ ID NO: 3).
152. The pharmaceutical composition for use as in any one of embodiments 149 to 151, wherein the first antigen-binding arm that binds CD3 comprises the VH1 of SEQ ID NO: 8 and the VL1 of SEQ ID NO: 7.
153. The pharmaceutical composition for use as in any one of embodiments 149 to 152, wherein the second antigen-binding arm that binds GPRC5D comprises a HCDR1 comprising the amino acid sequence of GFSLTNIRMSVS (SEQ ID NO: 12), HCDR2 comprising the amino acid sequence of HIFSNDEKS (SEQ ID NO: 13), and a HCDR3 comprising the amino acid sequence of MRLPYGMDV (SEQ ID NO: 14); and a LCDR1 comprising the amino acid sequence of RSSQSLVHSDGNTYLS (SEQ ID NO: 9), a LCDR2 comprising the amino acid sequence of KISNRFF (SEQ ID NO: 10), and a LCDR3 comprising the amino acid sequence of MQATQFPHT (SEQ ID NO: 11).
154. The pharmaceutical composition for use as in any one of embodiments 149 to 153, wherein the second antigen-binding arm that binds GPRC5D comprises the VH2 of SEQ ID NO: 16 and the VL2 of SEQ ID NO: 15.
155. The pharmaceutical composition for use as in any one of embodiments 149 to 154, wherein the third antigen-binding arm that binds BCMA comprises a HCDR1 comprising the amino acid sequence of GFTFSSYAMS (SEQ ID NO: 20), a HCDR2 comprising the amino acid sequence of AISGSGGSTY (SEQ ID NO: 21), and a HCDR3 comprising the amino acid sequence of DEGYSSGHYYGMDV (SEQ ID NO: 22); and a LCDR1 comprising the amino acid sequence of RASQSISSSFLT (SEQ ID NO: 17), a LCDR2 comprising the amino acid sequence of GASSRAT (SEQ ID NO: 18), and a LCDR3 comprising the amino acid sequence of QHYGSSPMYT (SEQ ID NO: 19).
156. The pharmaceutical composition for use as in any one of embodiments 149 to 155, wherein the third antigen-binding arm that binds BCMA comprises the VH3 of SEQ ID NO: 24 and the VL3 of SEQ ID NO: 23.
157. The pharmaceutical composition for use as in any one of embodiments 149 to 156, wherein the first antigen-binding arm that binds CD3 comprises the HCDR1, the HCDR2, the HCDR3, the LCDR1, the LCDR2 and the LCDR3 of SEQ ID NOs: 4, 5, 6, 1, 2, 3, respectively; the second antigen-binding arm that binds GPRC5D comprises the HCDR1, the HCDR2, the HCDR3, the LCDR1, the LCDR2 and the LCDR3 of SEQ ID NOs: 12, 13, 14, 9, 10 and 11, respectively; and the third antigen-binding arm that binds BCMA comprises the HCDR1, the HCDR2, the HCDR3, the LCDR1, the LCDR2 and the LCDR3 of SEQ ID NOS: 20, 21, 22, 17, 18 and 19, respectively.
158. The pharmaceutical composition for use as in any one of embodiments 149 to 157, wherein the first antigen-binding arm that binds CD3 comprises the VH1 of SEQ ID NO: 8 and the VL1 of SEQ ID NO: 7; the second antigen-binding arm that binds GPRC5D comprises the VH2 of SEQ ID NO: 16 and the VL2 of SEQ ID NO: 15; and the third antigen-binding arm that binds BCMA comprises the VH3 of SEQ ID NO: 24 and the VL3 of SEQ ID NO: 23.
159. The pharmaceutical composition for use as in any one of embodiments 149 to 158, wherein the BCMA×GPRC5D×CD3 trispecific antibody comprises a first antigen-binding arm that binds to an epitope on cluster of differentiation 3 (CD3), a second antigen-binding arm that binds to an epitope on G-protein coupled receptor family C group 5 member D (GPRC5D), and a third antigen-binding arm that binds to an epitope on B cell maturation antigen (BCMA), wherein the first antigen-binding arm comprises a heavy chain (HC1) polypeptide and a light chain (LC) polypeptide, wherein the heavy chain (HC1) polypeptide further comprises the second antigen-binding arm, wherein the trispecific antibody, or the trispecific binding fragment thereof, further comprises a single polypeptide comprising the third antigen-binding arm.
160. The pharmaceutical composition for use as in embodiment 159, wherein the HC1 of the first antigen-binding arm comprises the amino acid sequence of SEQ ID NO: 29.
161. The pharmaceutical composition for use as in embodiments 159 or 160, wherein the LC of the first antigen-binding arm comprises the amino acid sequence of SEQ ID NO: 30.
162. The pharmaceutical composition for use as in any one of embodiments 159 to 161, wherein the single polypeptide comprising the third antigen-binding arm comprises an amino acid sequence of SEQ ID NO: 31.
163. The pharmaceutical composition for use as in any one of embodiments 159 to 162, wherein the first antigen-binding arm comprises an HC1 comprising the amino acid sequence of SEQ ID NO: 29, and a LC comprising the amino acid sequence of SEQ ID NO: 30, and the single polypeptide comprising the third antigen-binding arm comprises the amino acid sequence of SEQ ID NO: 31.
164. The pharmaceutical composition for use as in any one of embodiments 159 to 163, wherein the BCMA×GPRC5D×CD3 trispecific antibody or trispecific binding fragment thereof is an (human) IgG1 isotype.
165. The pharmaceutical composition for use as in any one of embodiments 159 to 164, wherein the BCMA×GPRC5D×CD3 trispecific antibody or trispecific binding fragment thereof is for subcutaneous administration at a treatment dose of at least about 50 mg.
166. The pharmaceutical composition for use as in any one of embodiments 159 to 164, wherein the BCMA×GPRC5D×CD3 trispecific antibody or trispecific binding fragment thereof is for subcutaneous administration at a treatment dose of at least about 100 mg.
167. The pharmaceutical composition for use as in any one of embodiments 159 to 164, wherein the BCMA×GPRC5D×CD3 trispecific antibody or trispecific binding fragment thereof is for subcutaneous administration at a treatment dose of at least about 200 mg.
168. The pharmaceutical composition for use as in any one of embodiments 159 to 164, wherein the BCMA×GPRC5D×CD3 trispecific antibody or trispecific binding fragment thereof is for subcutaneous administration at a treatment dose of at least about 300 mg.
169. The pharmaceutical composition for use as in embodiments 139 to 168, wherein the pharmaceutical composition further comprises lenalidomide for oral administration at a dosage of 25 mg per day.
170. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg; and
   (d) the subject has received 1-3 lines of prior treatment.
171. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma., wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
   (d) the multiple myeloma is newly diagnosed multiple myeloma; and
   (e) the subject exhibits high-risk cytogenetic abnormalities selected from the group consisting of del(17p), t(4;14), t(14;16), and 1q21 gain/1q21 amplification.
172. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
   (d) the multiple myeloma is newly diagnosed multiple myeloma; and
   (e) the subject exhibits high-risk cytogenetic abnormalities selected from the group consisting of del(17p), t(4;14), t(14;16), and 1q21 gain/1q21 amplification.
173. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
   (d) the method further comprises administering lenalidomide to the subject, wherein lenalidomide is administered orally at a dose of about 25 mg daily for 21 days during a 28-day cycle, commencing on Cycle 2 Day 1;
   (e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
   (f) the multiple myeloma is newly diagnosed multiple myeloma.
174. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
   (d) the method further comprises administering lenalidomide to the subject, wherein lenalidomide is administered orally at a dose of about 25 mg daily for 21 days during a 28-day cycle, commencing on Cycle 2 Day 1;
   (e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
   (f) the multiple myeloma is newly diagnosed multiple myeloma.
175. The method of any one of embodiments 170-174, wherein the BCMA×GPRC5D×CD3 trispecific antibody treatment dose is about 50 mg, about 100 mg, about 200 mg, or about 300 mg.
176. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31, and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg; and
   (d) the subject has received 1-3 lines of prior treatment.
177. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
   (d) the multiple myeloma is newly diagnosed multiple myeloma; and
   (e) the subject exhibits high-risk cytogenetic abnormalities selected from the group consisting of del(17p), t(4;14), t(14;16), and 1q21 gain/1q21 amplification.
178. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
   (d) the multiple myeloma is newly diagnosed multiple myeloma; and
   (e) the subject exhibits high-risk cytogenetic abnormalities selected from the group consisting of del(17p), t(4;14), t(14;16), and 1q21 gain/1q21 amplification.
179. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered subcutaneously once every four weeks;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
   (d) the method further comprises administering lenalidomide to the subject, wherein lenalidomide is administered orally at a dose of about 25 mg daily for 21 days during a 28-day cycle, commencing on Cycle 2 Day 1;
   (e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
   (f) the multiple myeloma is newly diagnosed multiple myeloma.
180. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
   (a) the BCMA×GPRC5D×CD3 trispecific antibody is a BCMA×GPRC5D×CD3 trispecific antibody comprising:
      (i) a HC1 comprising the amino acid sequence of SEQ ID NO: 29;
      (ii) a LC comprising the amino acid sequence of SEQ ID NO: 30, and
      (iii) a HC2 comprising an amino acid sequence of SEQ ID NO: 31,
      and wherein the BCMA×GPRC5D×CD3 trispecific antibody is administered once every four weeks;
   (b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
   (c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg,
   (d) the method further comprises administering lenalidomide to the subject, wherein lenalidomide is administered orally at a dose of about 25 mg daily for 21 days during a 28-day cycle, commencing on Cycle 2 Day 1;
   (e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
   (f) the multiple myeloma is newly diagnosed multiple myeloma.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Molecule Design, Sequence, and Structure of BGCB491 Trispecific Antibody

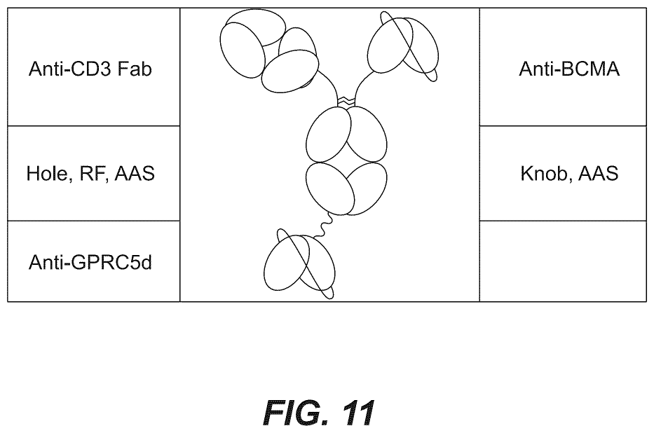

BGCB491 is an immunoglobulin (Ig) G1 trispecific antibody that can bind simultaneously or independently to the epsilon subunit of the cluster of differentiation 3 receptor complex (CD3ε) (Uniprot ID: P07766) on T lymphocytes (T cells), and to GPRC5D (G-protein coupled receptor family C group 5 member D, Uniprot ID: Q9NZD1) and BCMA (B cell maturation antigen, TNFRSF17, Uniprot ID: Q02223) on tumor cells. The antibody features mutations of L234A, L235A, and D265S in the constant region (Fc) to abolish interaction with Fc receptors and heterodimerization is enhanced using the knobs-into-holes platform mutations 1. The anti-CD3E “hole” chain also featured “RF” mutations (H435R, Y436F) to disrupt protein A binding of monomeric and homodimerized hole chains 2. The molecule comprises an anti-CD38 Fab region on the “hole, RF” chain and an anti-GPRC5D scFv v-region fused onto the C-terminus. The “knob” chain features the anti-BCMA scFv. The trispecific antibody was developed to evaluate the therapeutic potential of dual tumor targeting GPRC5D and BCMA and CD3 for T cell redirection. An illustration of BGCB491 is depicted in FIG. 11.

The trispecific BGCB491 antibody was generated by co-expression of the anti-CD3 heavy chain (HC) A fused to the C-terminal anti-GPRC5D scFv and anti-CD3 light chain (LC) with the anti-BCMA scFv fused to “knob” containing heavy chain B. The anti-CD3 variable region (VR000017350) was discovered by immunizing transgenic humanized rats [Omni Rat (OMT™)] with recombinant CD3 protein. The anti-GPRC5d variable region (VR000038761) featured in BGCB491 is derived from the mAb GC5B680, discovered by immunizing transgenic humanized mice [Ablexis] with DNA encoding GPRC5d. The parent v-region (VR000029832) contained an “NSS” motif in the HC framework 3 region which presented a risk for N-linked glycosylation and which represented a mutation from the IGHV2-26*01 germline. The site was mutated back to the germline sequence “STS” to eliminate the risk for N-linked glycosylation, and this change resulted in the final variable region VR000038761. The anti-BCMA variable region (VR000003260) featured in BGCB491 is derived from the mAb BCMB519, discovered by immunizing transgenic humanized mice [Ablexis] with recombinant BCMA protein. No further modifications were made to this v-region. Both the anti-GPRC5D and anti-BCMA v-regions were formatted as single-chain fragment variable (scFv) in the final molecule.

The amino acid sequence for the BGCB491 heavy chains and light chain, as deduced from the cDNA sequence (SEQ ID Nos: 165, 166, and 167) of BGCB491 and confirmed by peptide mapping and mass spectrometry is shown below (Table 2). The complementarity-determining regions (CDRs) are defined according to ABM numbering. The Gln residue at position 1 of BGCB491 heavy chain 1, Gln residue at position 1 of BGCB491 light chain, and Glu residue at position 1 of BGCB491 heavy chain 2 constitute the N-termini of the mature chains. Both heavy chains comprising BGCB491 IgG1 AAS have the following point mutations: L234A, L235A, and D265S. Heavy chain 1 from BGCB491 features the “hole” mutations: T366S, L368A, Y407V and the RF mutations: H435R, Y436F, while heavy chain 2 features the “knob” mutation: T366W. The knobs-into-holes mutations promote heterodimerization of the Fc³. The “RF” mutations disrupt binding to protein A. The mutations for FcγR receptor silencing (AAS) and the knobs-into-holes mutations are underlined in the sequences below.

TABLE 2
Amino Acid Sequence of BGCB491

| | | SEQ |
|---|---|---|
| Area | AA Sequence | ID NO. |
| Light | QSALTQPASV SGSPGQSITI SCTGTSSNIG | 30 |
| Chain 1 | TYKFVSWYQQ HPDKAPKVLL YEVSKRPSGV | |
| | SSRFSGSKSG NTASLTISGL QAEDQADYHC | |
| | VSYAGSGTLL FGGGTKLTVL GQPKAAPSVT | |
| | LFPPSSEELQ ANKATLVCLI SDFYPGAVTV | |
| | AWKADSSPVK AGVETTTPSK QSNNKYAASS | |
| | YLSLTPEQWK SHRSYSCQVT HEGSTVEKTV | |
| | APTECS | |
| Heavy | QVQLQQSGPR LVRPSQTLSL TCAISGDSVF | 29 |
| Chain 1 | NNNAAWSWIR QSPSRGLEWL GRTYYRSKWL | |
| | YDYAVSVKSR ITVNPDTSRN QFTLQLNSVT | |
| | PEDTALYYCA RGYSSSFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGG | |
| | PSVFLFPPKP KDTLMISRTP EVTCVVVSVS | |
| | HEDPEVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLSC AVKGFYPSDI AVEWESNGQP | |
| | ENNYKTTPPV LDSDGSFFLV SKLTVDKSRW | |
| | QQGNVFSCSV MHEALHNRFT QKSLSLSPGK | |
| | GGGGSGGGGS GGGGSGGGGS DIVMTQTPLS | |
| | SPVTLGQPAS ISCRSSQSLV HSDGNTYLSW | |
| | LQQRPGQPPR LLIYKISNRF FGVPDRFSGS | |
| | GAGTDFTLKI SRVEAEDVGV YYCMQATQFP | |
| | HTFGQGTKLE IKGGSEGKSS GSGSESKSTG | |
| | GSQVTLKESG PVLVKPTETL TLTCTVSGFS | |
| | LTNIRMSVSW IRQPPGKALE WLAHIFSNDE | |
| | KSYSTSLKSR LTISRDTSKS QVVLTLTNVD | |
| | PVDTATYYCA RMRLPYGMDV WGQGTTVTVS | |
| | S | |
| Heavy | EIVLTQSPGT LSLSPGERAT LSCRASQSIS | 31 |
| Chain 2 | SSFLTWYQQK PGQAPRLLIY GASSRATGIP | |
| | DRFSGGGSGT DFTLTISRLE PEDFAVYYCQ | |
| | HYGSSPMYTF GQGTKLEIKG GSEGKSSGSG | |
| | SESKSTGGSE VQLLESGGGL VQPGGSLRLS | |
| | CAASGFTFSS YAMSWVRQAP GKGLEWVSAI | |
| | SGSGGSTYYA DSVKGRFTIS RDNSKNTLYL | |
| | QMNSLRAEDT AVYYCAKDEG YSSGHYYGMD | |
| | VWGQGTTVTV SSEPKSSDKT HTCPPCPAPE | |
| | AAGGPSVFLF PPKPKDTLMI SRTPEVTCVV | |
| | VSVSHEDPEV KFNWYVDGVE VHNAKTKPRE | |
| | EQYNSTYRVV SVLTVLHQDW LNGKEYKCKV | |
| | SNKALPAPIE KTISKAKGQP REPQVYTLPP | |
| | SREEMTKNQV SLWCLVKGFY PSDIAVEWES | |
| | NGQPENNYKT TPPVLDSDGS FFLYSKLTVD | |
| | KSRWQQGNVF SCSVMHEALH NHYTQKSLSL | |
| | SPGK | |

### Example 2: In Vitro Study of Combinations of BGCB491 and Daratumumab in an In Vitro Cytotoxicity Assay with H929 Myeloma Cell Line and Effector Cells from Healthy Human Donors

BGCB491 (GPRC5D×BCMA×CD3) is a trispecific antibody that binds to BCMA, GPRC5D, and the epsilon subunit of the CD3 T cell receptor complex. BGCB491 draws BCMA- and GPRC5D-expressing malignant multiple myeloma (MM) cells in close proximity to T cells expressing CD3, leading to T-cell activation and the release of cytotoxic factors such as perforin and granzymes, resulting in subsequent lysis of tumor cells.

Daratumumab is an antibody targeting the cell surface protein CD38, a multifunctional transmembrane glycoprotein involved in cellular activation, proliferation, and differentiation of MM cells (Costa, Federica et al. CD38 Expression by Myeloma Cells and Its Role in the Context of Bone Marrow Microenvironment: Modulation by Therapeutic Agents. Cells vol. 8,12 1632. 13 Dec. 2019). Daratumumab exerts its function through direct cytotoxicity against tumor cells as well as modulating various immune compartments through multiple mechanisms. It induces complement-dependent cytotoxicity (CDC), antibody-dependent cellular cytotoxicity (ADCC) and antibody-dependent cellular phagocytosis (ADCP) of the target cells mediated by natural killer (NK) cells, granulocytes and macrophages (de Weers M, Tai Y T, van der Veer M S, Bakker J M, Vink T, Jacobs D C, Oomen L A, Peipp M, Valerius T, Slootstra J W, Mutis T, Bleeker W K, Anderson K C, Lokhorst H M, van de Winkel J G, Parren P W. Daratumumab, a novel therapeutic human CD38 monoclonal antibody, induces killing of multiple myeloma and other hematological tumors. J Immunol. 2011 Feb. 1; 186 (3): 1840-8.).

### Peripheral Blood Mononuclear Cell (PBMC)-Mediated Immune Killing Assays.

### Cell Lines and Primary Cells

Human cell lines were obtained from Janssen Spring House Cell Banking Services. All cell lines, NCI-H929 cells and PBMCs of donors R100604, EN01000804 and SD01000472, were cultured at 37° C., 5% CO2 in complete culture medium RPMI 1640 with GlutaMAX and 10% heat inactivated fetal bovine serum (HI-FBS). All cell lines tested negative for mycoplasma.

### Trispecific Antibody (BGCB491 Preparation)

BGCB491 was prepared at a 10× concentration in complete media to yield 1× when 20 μL was added for a total volume of 200 μL in each well of a 96-well U-bottom plate. The volume in each well at this step will be 180 μL prior to the addition of daratumumab. Serial dilutions of BGCB491 trispecific antibody (1:3) with a final concentration of: 1, 0.3, 0.1, 0.03, 0.01, 0.003, 0.001, 0.0003, 0.0001, and 0.00003 nM were prepared in a multi-channel reservoir.

### Daratumumab Preparation

Daratumumab was prepared at a 10× concentration in complete media to yield 1× when 20 μL was added for a total volume of 200 μL in each well of a 96-well U-bottom plate. Serial dilutions of BGCB491: Daratumumab (1:3) with a final concentration of: 10, 3, 1, 0.3, 0.1, 0.03, 0.01 nM were prepared in a multi-channel reservoir.

### Preparation of Effector and Target Cells for Co-Treatment Experiments.

Immune killing assays were performed to determine the cytotoxic effect of the combination of daratumumab with BGCB491. Within the PBMC, NK cells were the effector cells for daratumumab-mediated ADCC, and T cells mediated BGCB4912 cytotoxicity. Briefly, on day 0, PBMCs were thawed in complete media (RPMI GlutaMAX+10% v/v HI FBS). Frozen vials were wiped with 70% isopropanol and thawed in a 37° C. water bath by gently swirling the vial. After a small amount of ice remained, vials were wiped again with 70% isopropanol and transferred into biosafety cabinet. 10 mL of complete media were transferred to a 15 mL tube and the components of the vials were transferred to the 15 mL tube. Cells were centrifuged at 400×g for 5 minutes at room . . . temperature. After centrifugation, the supernatant was discarded, and the cell pellet was resuspended with 10 mL of complete media and an aliquot was used to count cells and assess viability by trypanblue exclusion using the Vi-CELL XR Cell Viability Analyzer (Beckman Coulter). PBMCs were plated first at a concentration of 90,000 cells per 80 μL of complete media in each well of 96-well U-bottom plates.

Tumor cells H929 were collected from a 37° C., 5% CO2 incubator. Cells were counted and viability was determined using Trypan blue exclusion with the Vi-CELL XR Cell Viability Analyzer. Cell suspensions were removed from culture flasks and centrifuged at 400×g for 5 minutes at room temperature. The cells were washed once in 10 mL sterile PBS before being stained with a CellTrace Dye. Briefly, a 5 mM stock of the CellTrace CFSE dye was prepared by adding 18 μL of sterile DMSO to the vial containing 50.17 μg CFSE and allowing to sit at room temperature in the dark for 30 minutes and then diluted to 1:10,000 in PBS. One ml of the diluted CFSE per 10 million target cells was added and incubated for 8 minutes at room temperature in the dark. At the end of incubation, staining was quenched by adding 5×RPMI GlutaMAX+20% v/v HI FBS media. The CellTrace dye-loaded cells were washed three times with RPMI GlutaMAX+20% v/v HI FBS media then resuspended in 10 mL complete media and recounted using the Vi-CELL XR Cell Viability Analyzer (Beckman Coulter). Target cells were then added at a density of 30,000 cells per 80 μl of complete media in each well of 96-well U-bottom plates (3:1 effector (E):target (T) ratio).

### Flow Cytometry

After 48-hour incubation, the cells were spun down and supernatant was saved at-80° C. for cytokine assessment by MSD. The cells were washed in 200 μL 1×PBS and spun down at 400×g for 5 minutes at room temperature. The supernatant was removed and 50 μL of 1:2,000 Live/Dead Viability stain in 1×PBS was added and incubated for 30 minutes with shaking at room temperature in the dark. Live/Dead Fixable Near-IR Dead Cell Stain Kit was prepared following manufacturer's instructions by adding 50 μl of sterile DMSO per vial and allowing to sit at room temperature in the dark for 30 minutes and then diluted to 1:2000 in PBS. Following the 30-minute incubation, cells were washed with 200 μL BD Stain Buffer (containing BSA) and spun down at 400×g for 5 minutes. Supernatant was discarded and the cells were then resuspended in 50 μL of 1:20 Human TruStain FcX diluted in BD Stain Buffer. The plates were incubated for 15 minutes with shaking at room temperature in the dark followed by one wash with 200 μL BD Stain Buffer (containing BSA) per well. Cells were then suspended in 50 μL of antibody cocktail made in brilliant stain buffer as described in the table below. Brilliant stain buffer was prepared using 200 μL Brilliant Stain Buffer Plus and 19.8 mL BD Stain Buffer (containing BSA).

Antibody cocktail incubation was 30 minutes at room temperature in the dark with shaking. After the incubation, cell plates were washed twice with 200 μL of BD Stain Buffer (containing BSA) and then resuspended in 110 μL of BD Stain Buffer (containing BSA) for acquisition on BD Fortessa Cell-Analyzer. Flow cytometry data was analyzed using FlowJo v10.9.0 and FACSApp (https://rconnect.jnj.com/facsApp/). Values were graphed in GraphPad Prism v9.0 [12]. Raw data files were exported from the BD instrument software and imported into FlowJo.

TABLE 3
Flow Cytometry Panel for T Cell Redirection Assay Using PBMCs and H929 Cells

| Marker | Channel | Clone | Manufacturer | Catalog number | Dilution |
|---|---|---|---|---|---|
| CD3 | BUV 395 | UCHT1 | BD | 563546 | 1:100 |
| CD25 | BV421 | M-A251 | Biolegend | 356114 | 1:400 |
| CD56 | BV510 | HCD56 | Biolegend | 318340 | 1:80 |
| CD19 | BV605 | HIB19 | Biolegend | 302244 | 1:300 |
| CD137 | BV650 | 4B4-1 | Biolegend | 309828 | 1:100 |
| CD8 | BV786 | RPA-T8 | Biolegend | 301046 | 1:600 |
| CFSE | CFSE | | ThermoFisher | C34554 | 1:10,000 |
| CD4 | PerCp Cy5.5 | RPA-T4 | Biolegend | 300530 | 1:100 |
| CD69 | PE | FN50 | Biolegend | 310906 | 1:100 |
| CD279 (PD1) | PE-Dazzle | EH12.2H7 | Biolegend | 329940 | 1:200 |
| CD16 | PE-Cy7 | 3G8 | Biolegend | 302016 | 1:600 |
| CD38 | Alexa 647 | HUMAB003 | Biolegend | 94899 | 1:100 |
| HLA-DR | Alexa 700 | L243 | Biolegend | 307626 | 1:20 |
| LIVE/DEAD | APC-Cy7 | | Life Technologies | L34976 | 1:2000 |

BUV, Brilliant UltraViolet; BV, Brilliant Violet; PerCP/Cy5.5, Peridinin-chlorophyll-protein complex/Cyanine5.5; PE, Phycoerythrin; PE-Cy7, Phycoerythrin-Cyanine7; APC, Allophycocyanin; APC-Cy7, Allophycocyanin-Cyanine7; CD, Cluster of Differentiation.

### Data Analysis

### EC50

EC50 Statistical analysis was performed using extra sum-of-squares F test with a p-value less than 0.05.

### Synergy Calculations

To model the dose response relationship of each monotherapy, a 4-parameter logistic (4PL) regression was employed, with a shared lower/upper asymptote (lower for increasing curves and upper for decreasing curves). Additionally, the lower asymptote was constrained to be greater or equal to zero. These 4PL regression models were then used to derive the expected effect of the combination assuming additivity under ‘HSA’, ‘Bliss’, ‘Alternative Loewe’ null models [13, 14]. Differences in mean observed combination effects and mean expected combination effects were compared and reported together with a bootstrapped confidence interval. These differences were reported as synergy or antagonism (depending on the direction of the difference relative to direction of dose response curve) if the 90% confidence interval of the difference did not include 0. Standard errors of the differences were estimated using R=5000 bootstrap replicates and assuming unequal variance of the monotherapy and combination data. Furthermore, the width of the confidence intervals were adjusted such that the family-wise error rate was controlled at 10% alpha level. All analyses were performed in R version 4.3.0 using the Synergy shiny app version 0.0.26.0.0.0.1708619195.

### Results

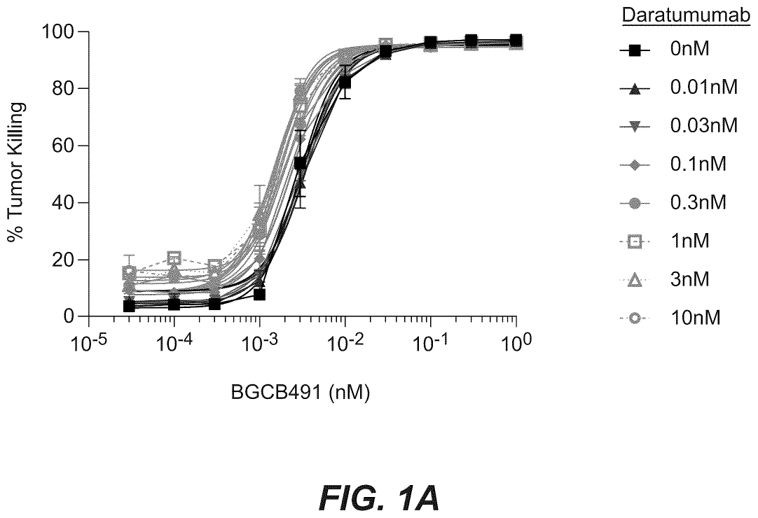

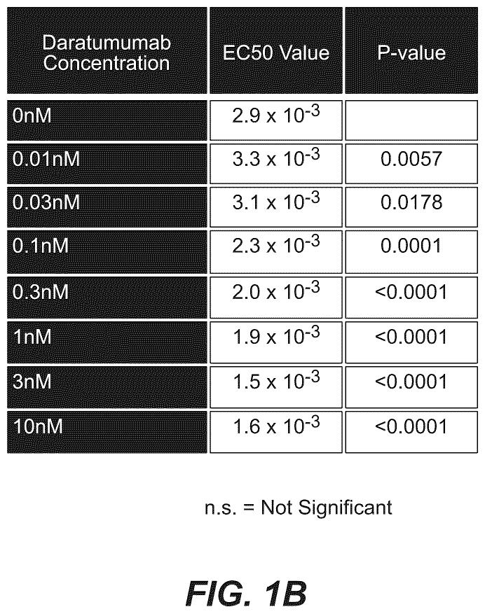

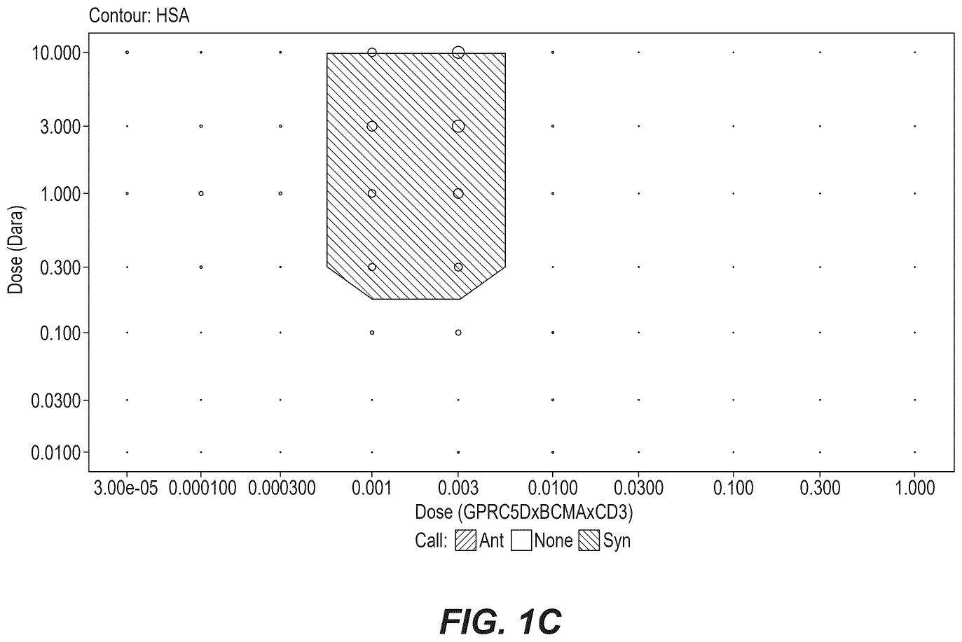

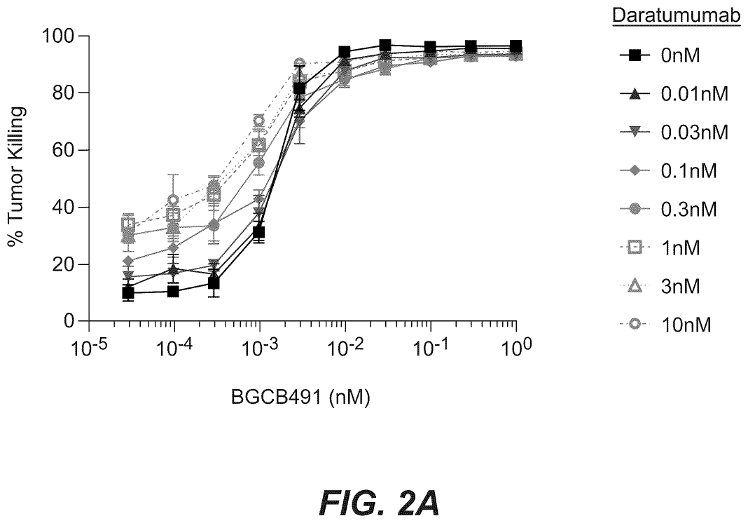

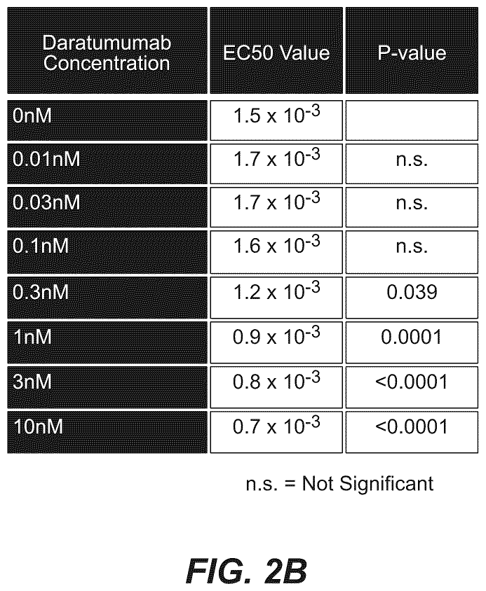

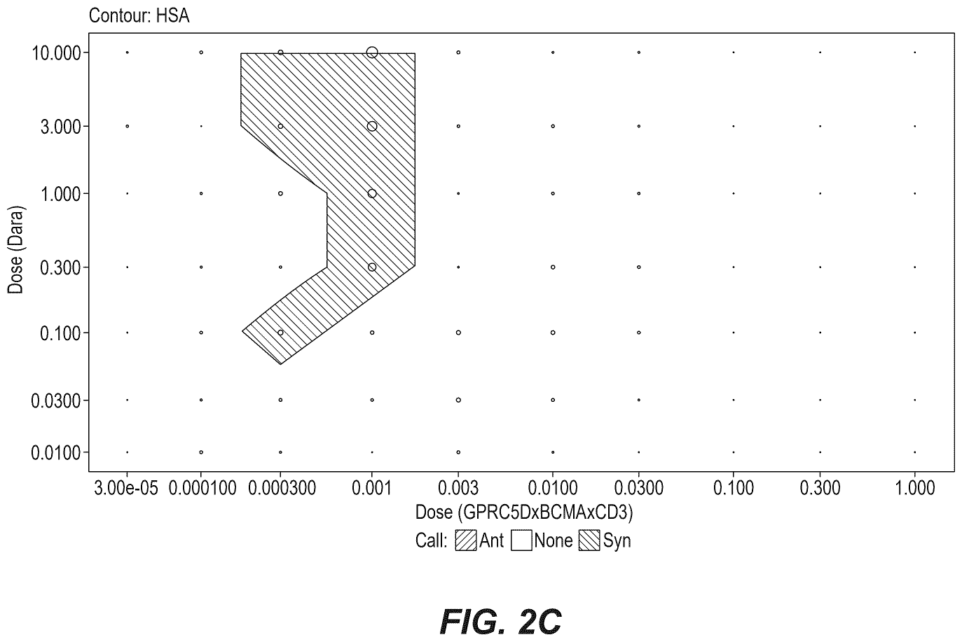

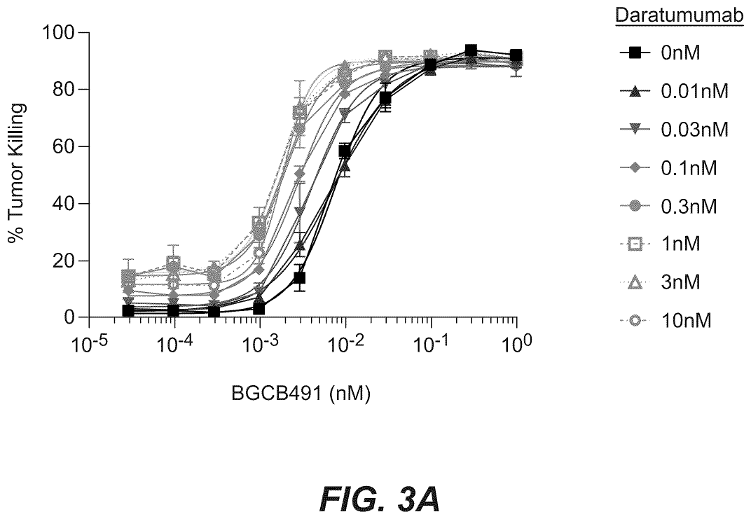

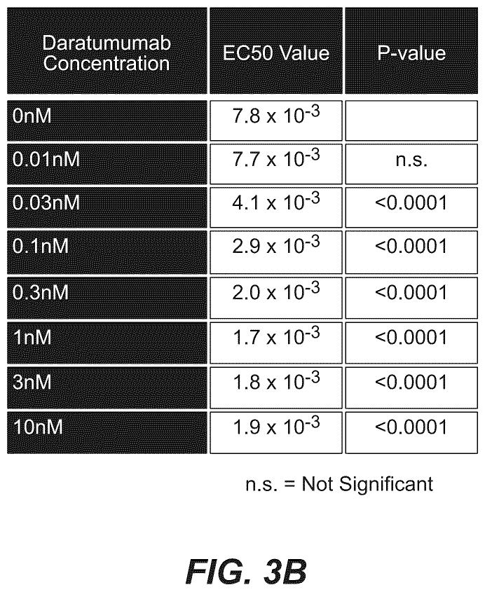

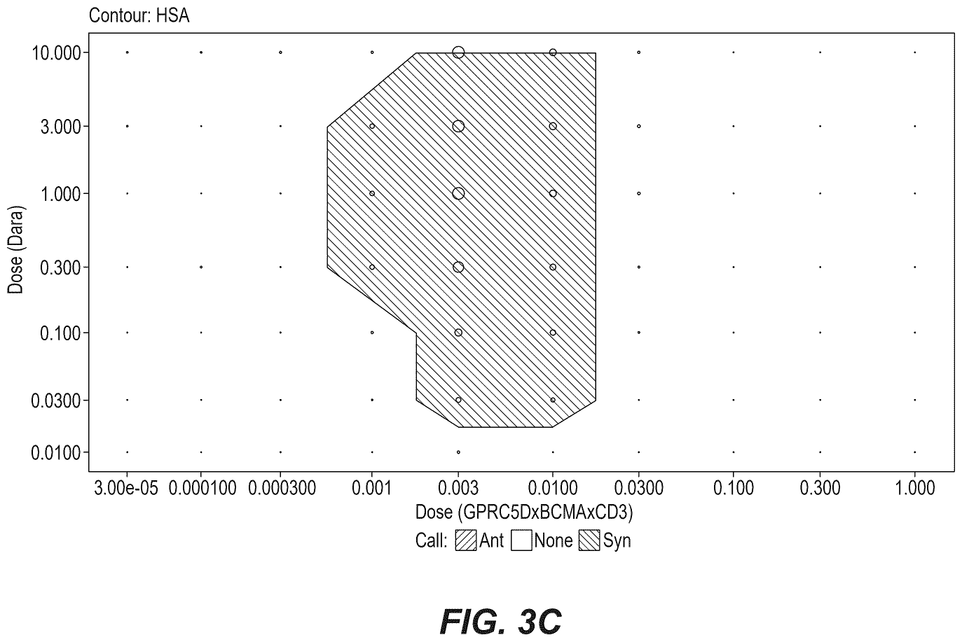

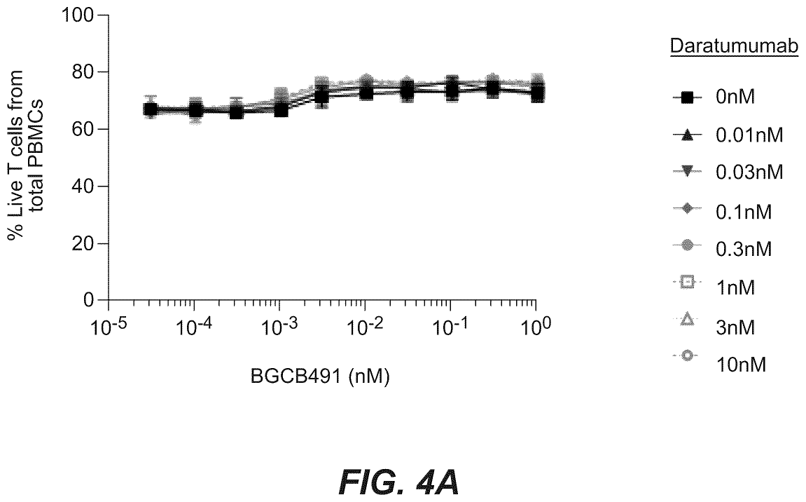

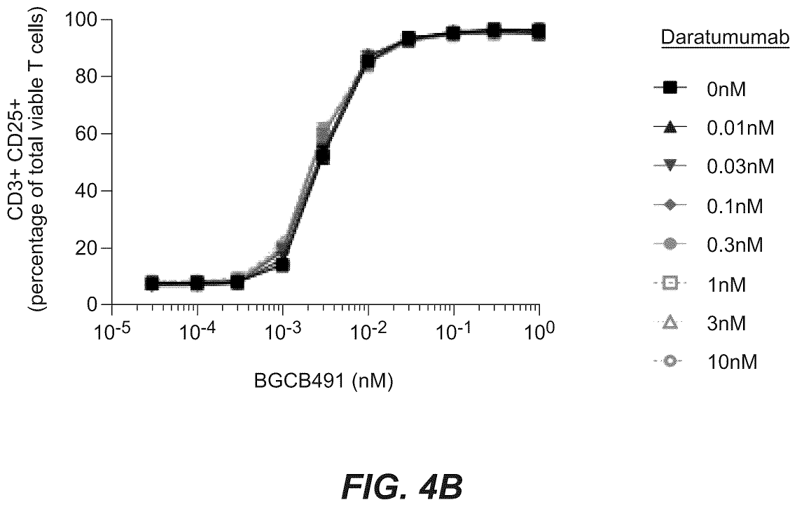

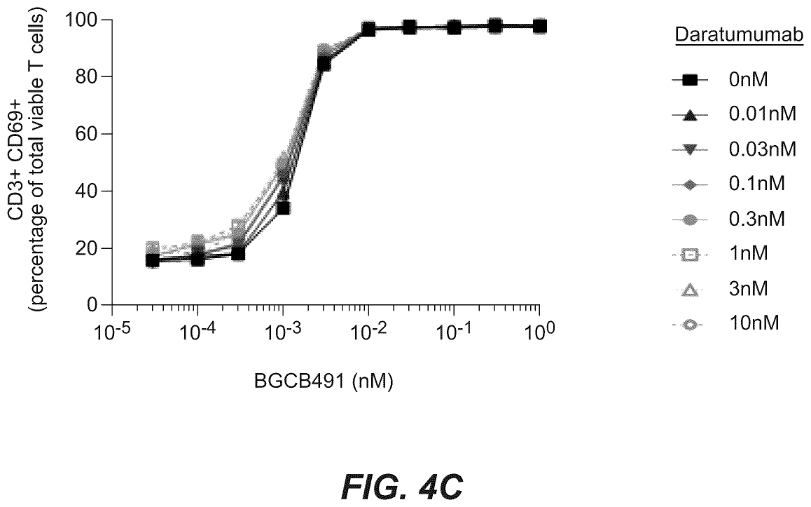

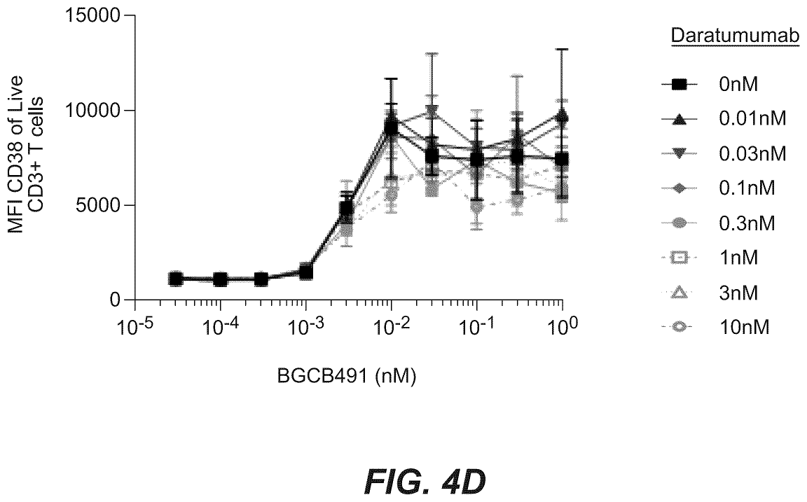

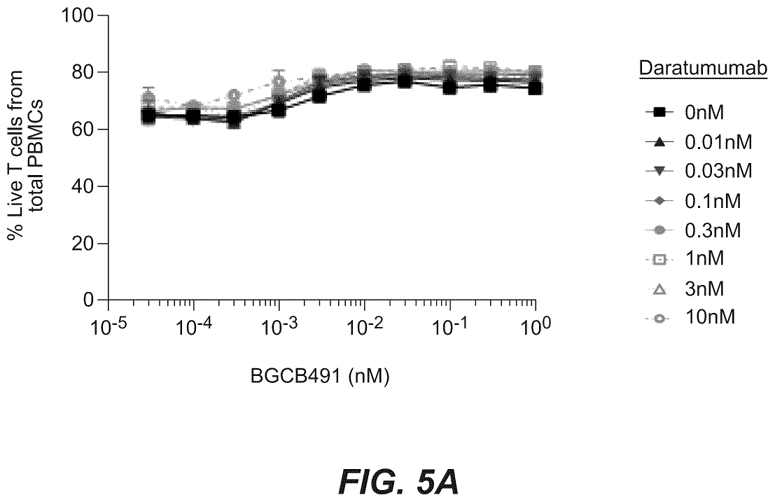

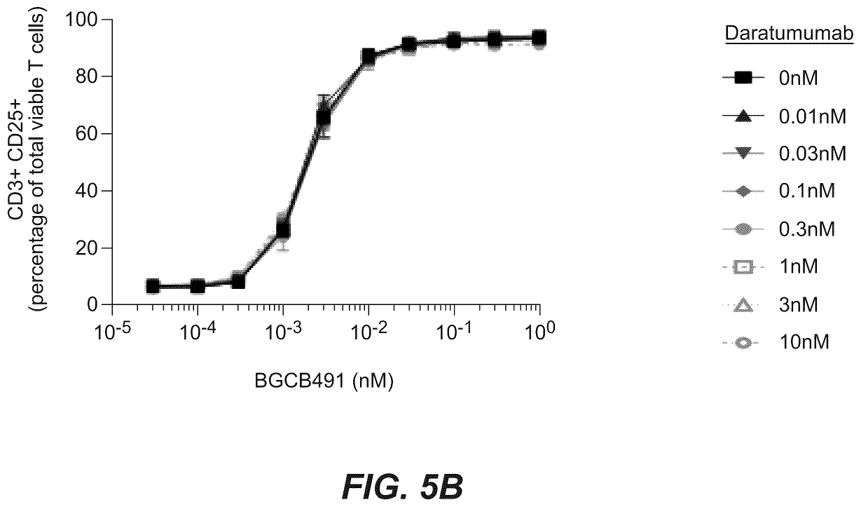

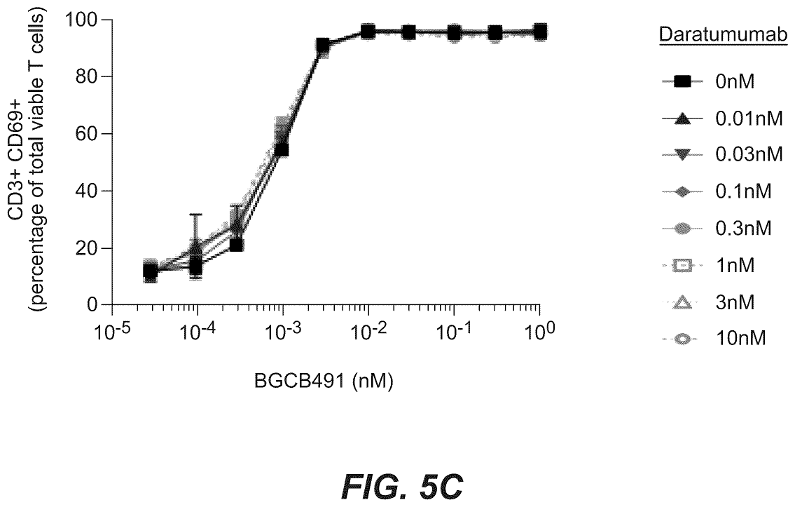

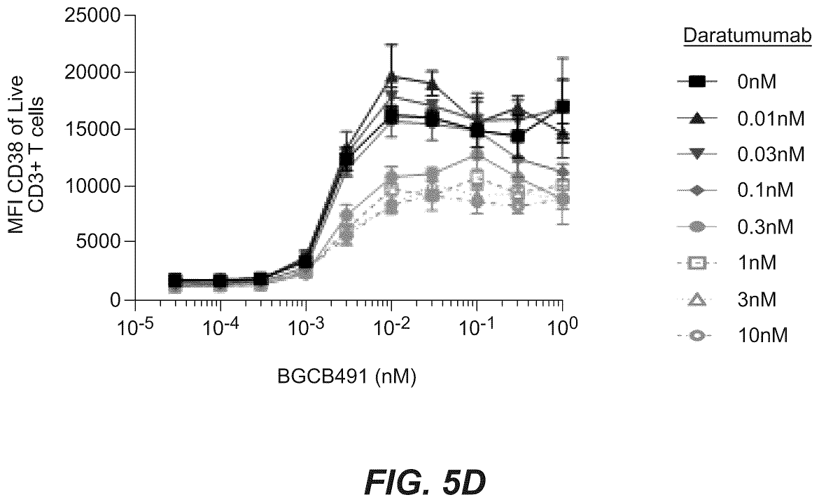

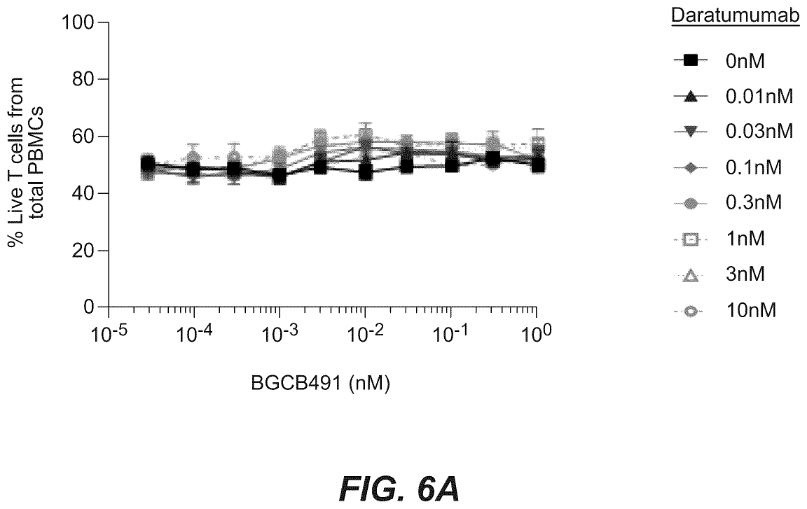

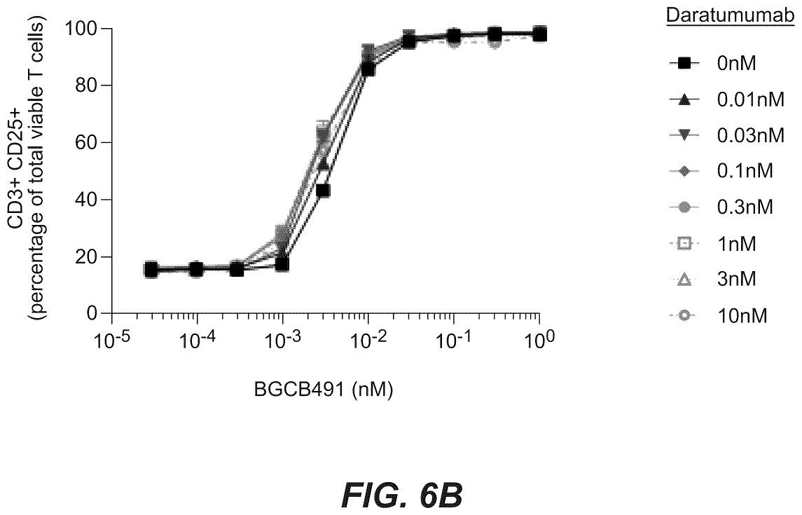

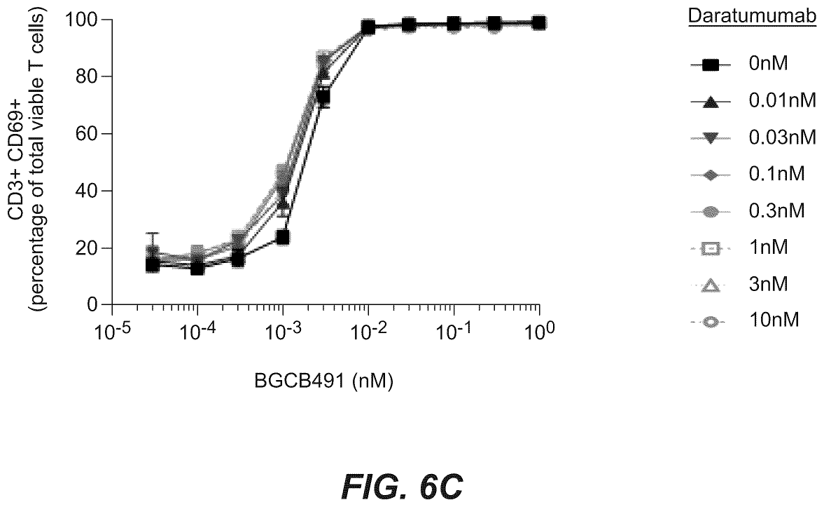

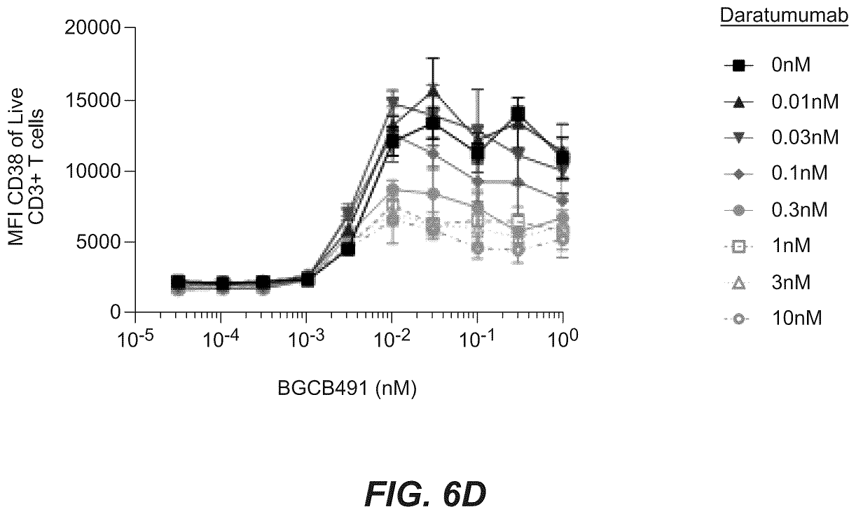

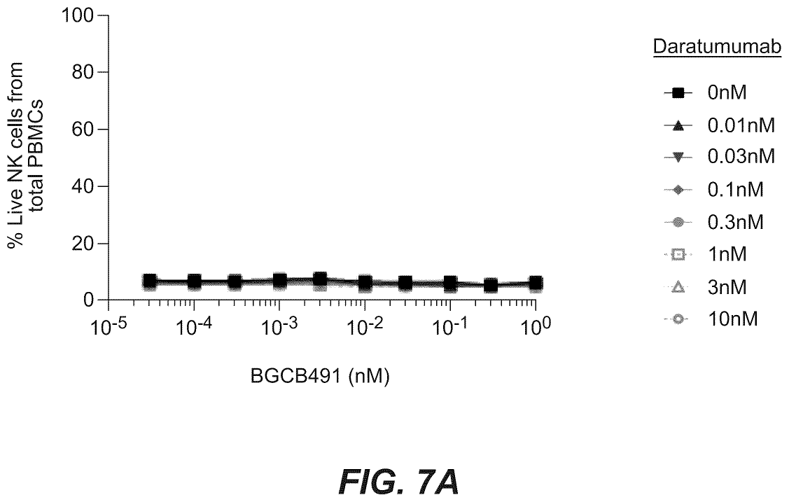

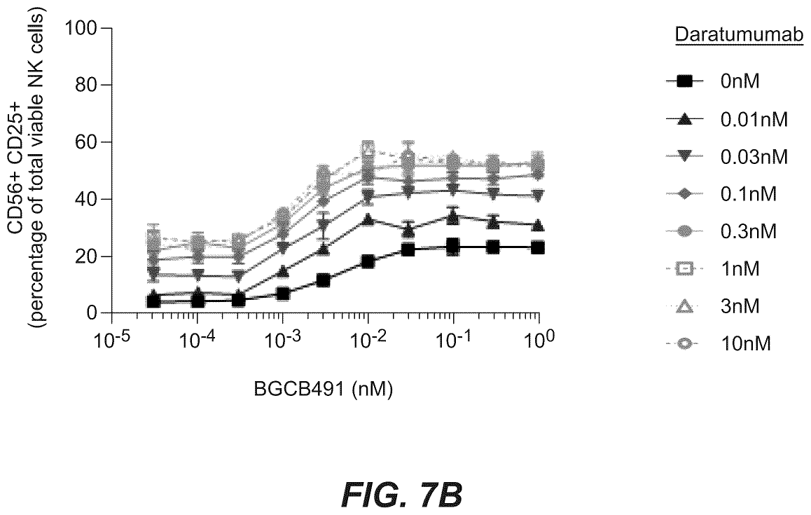

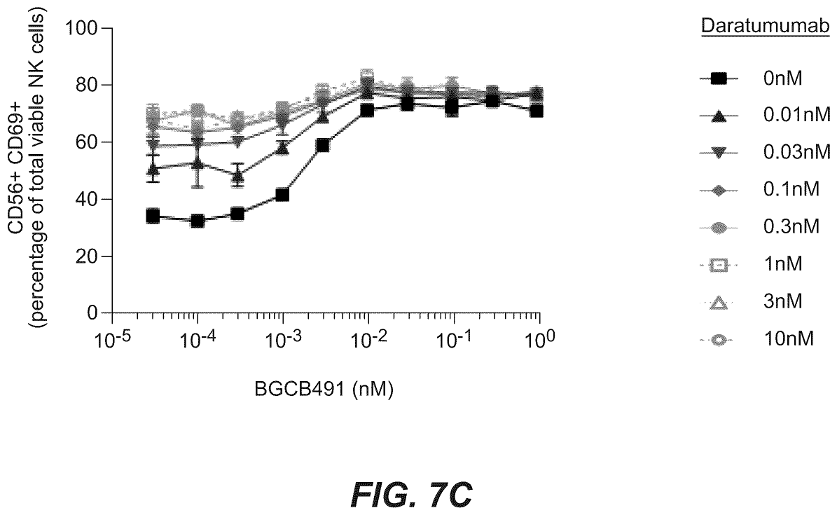

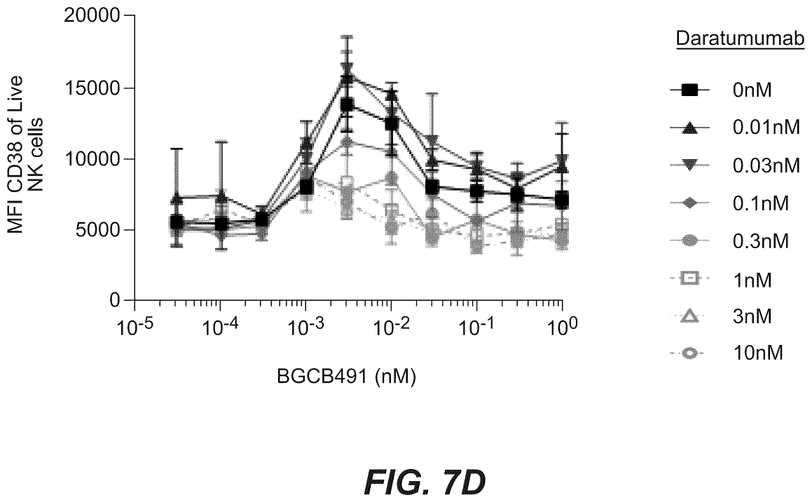

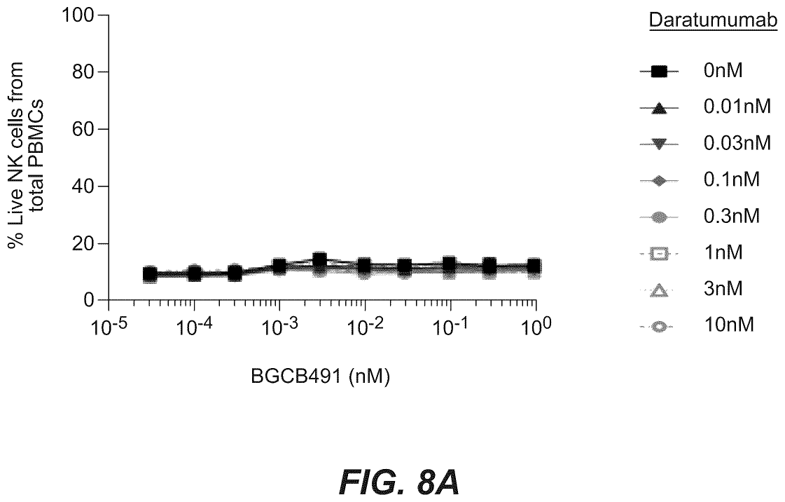

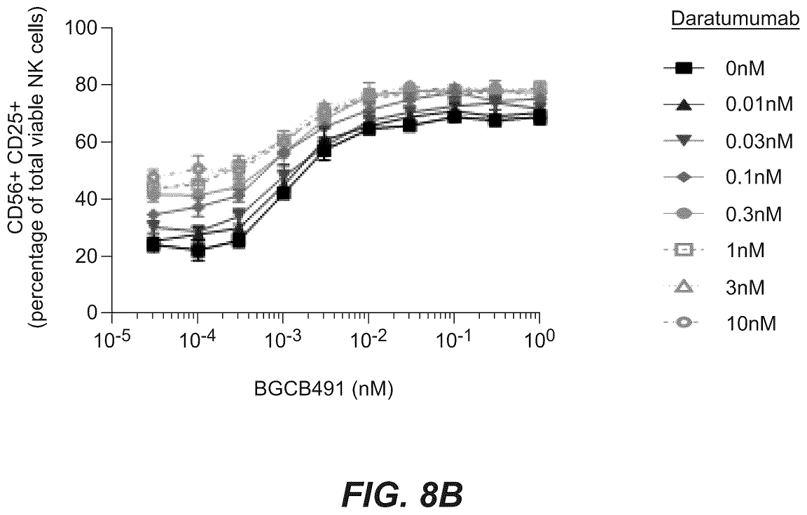

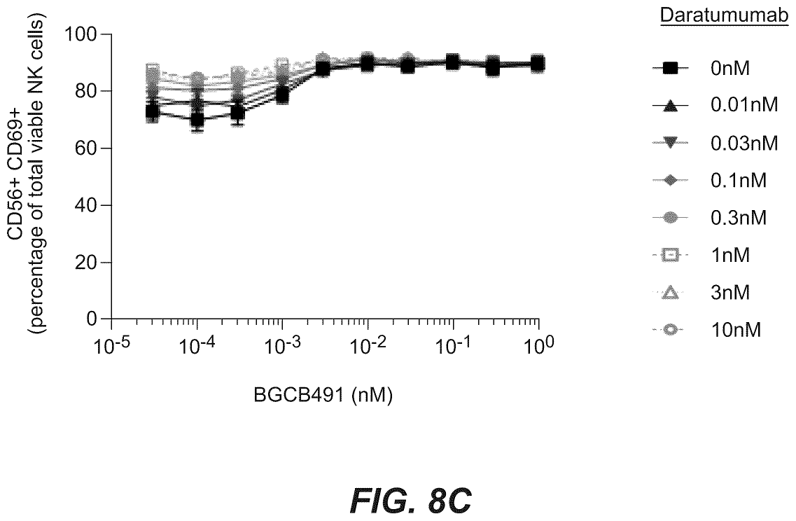

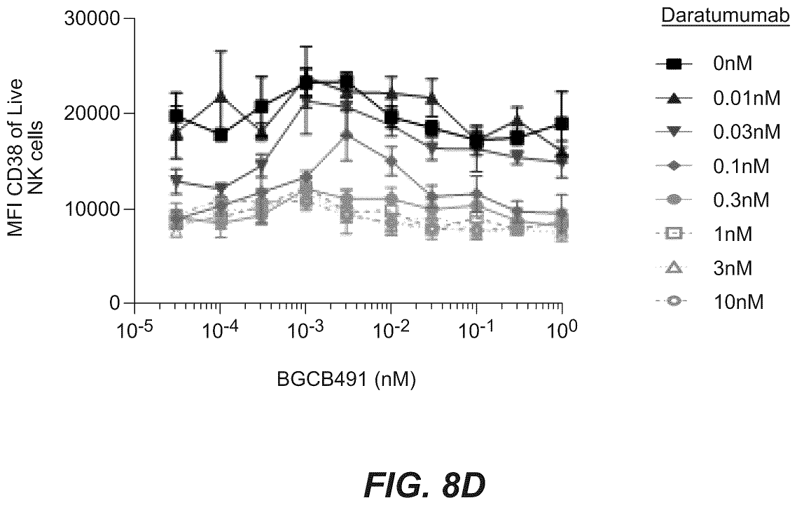

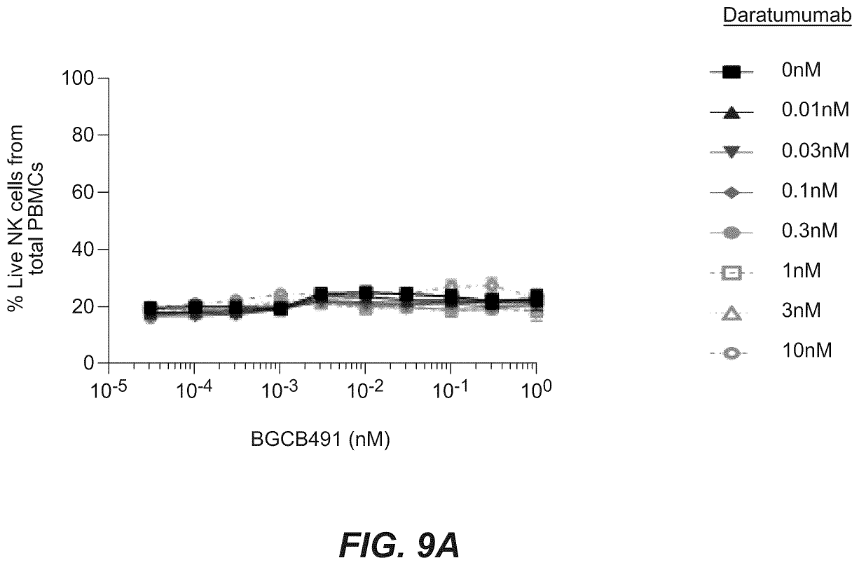

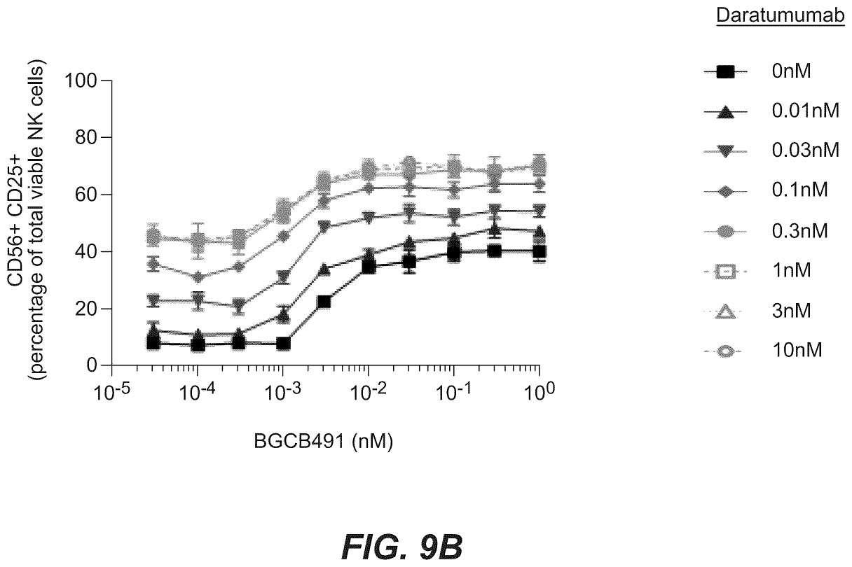

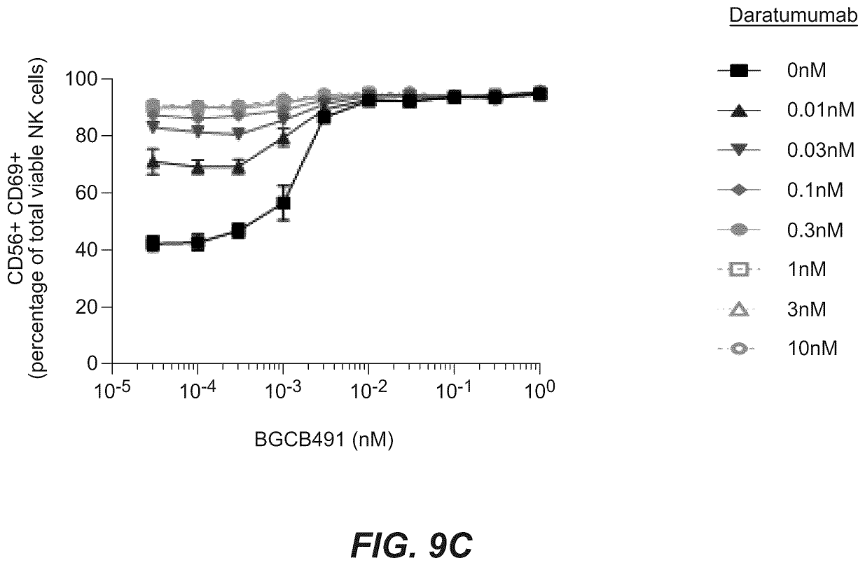

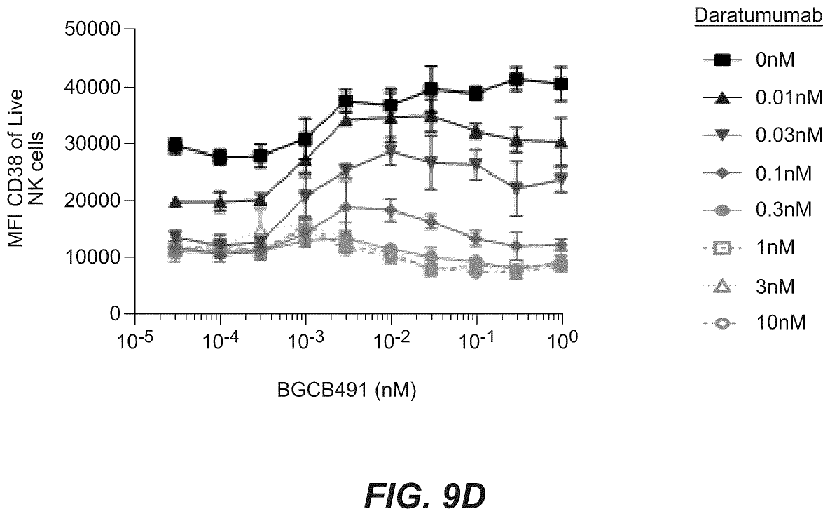

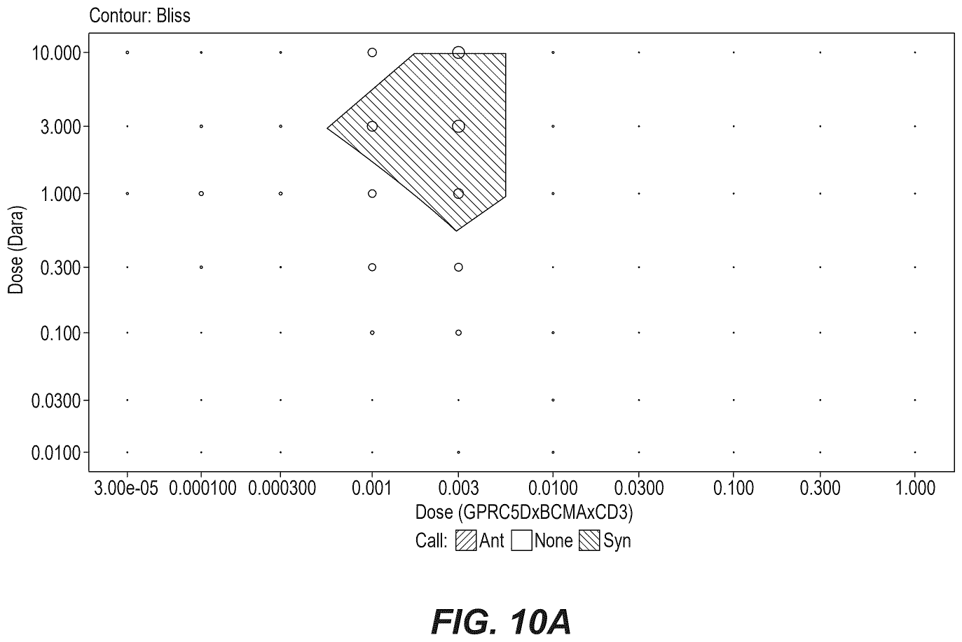

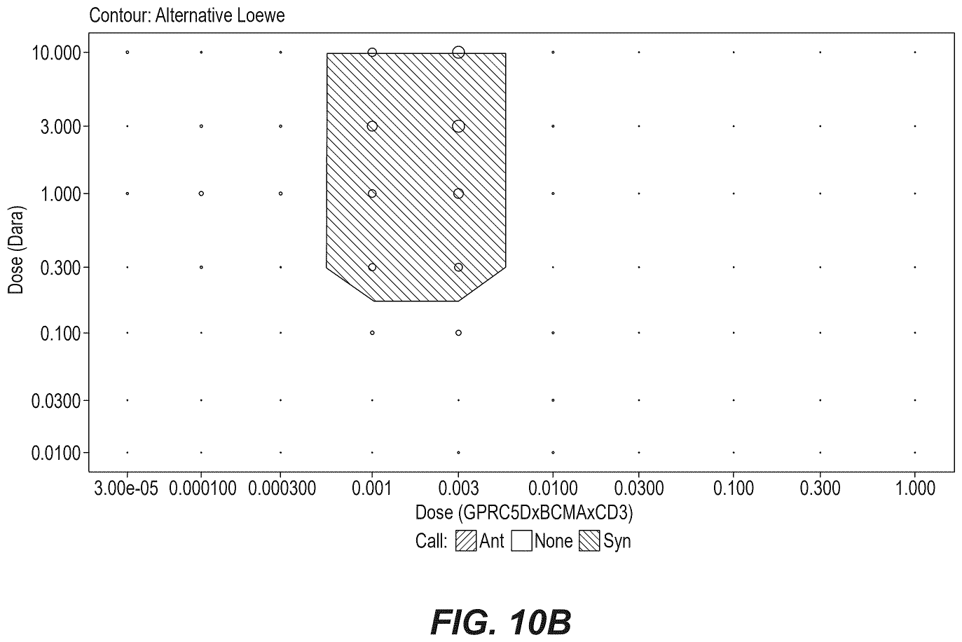

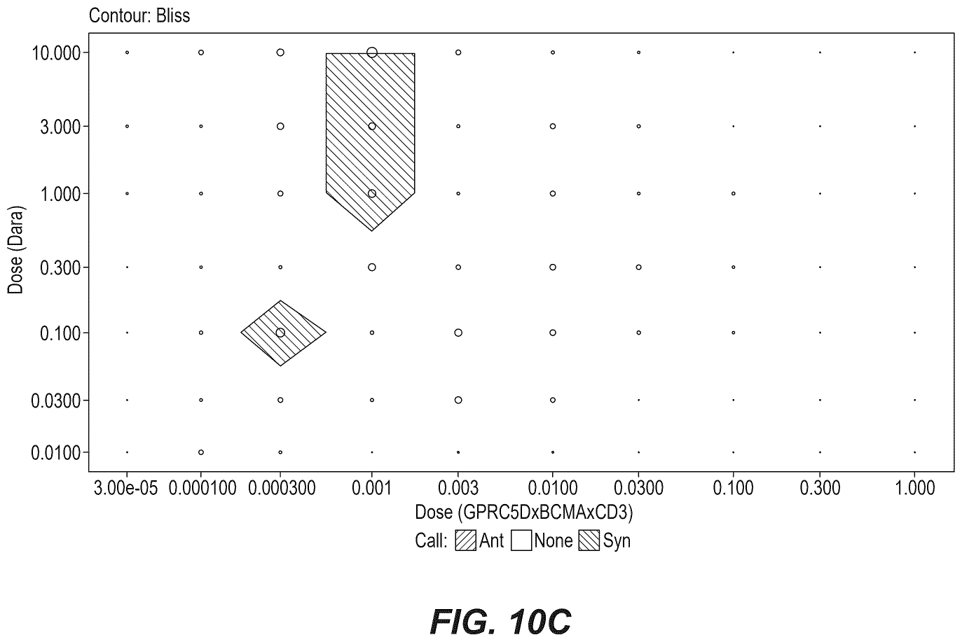

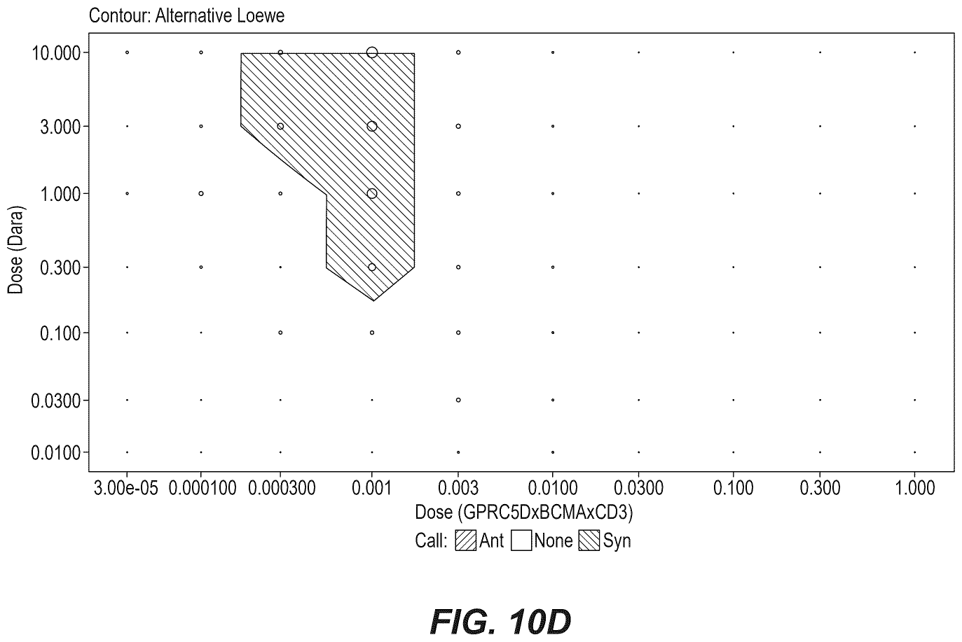

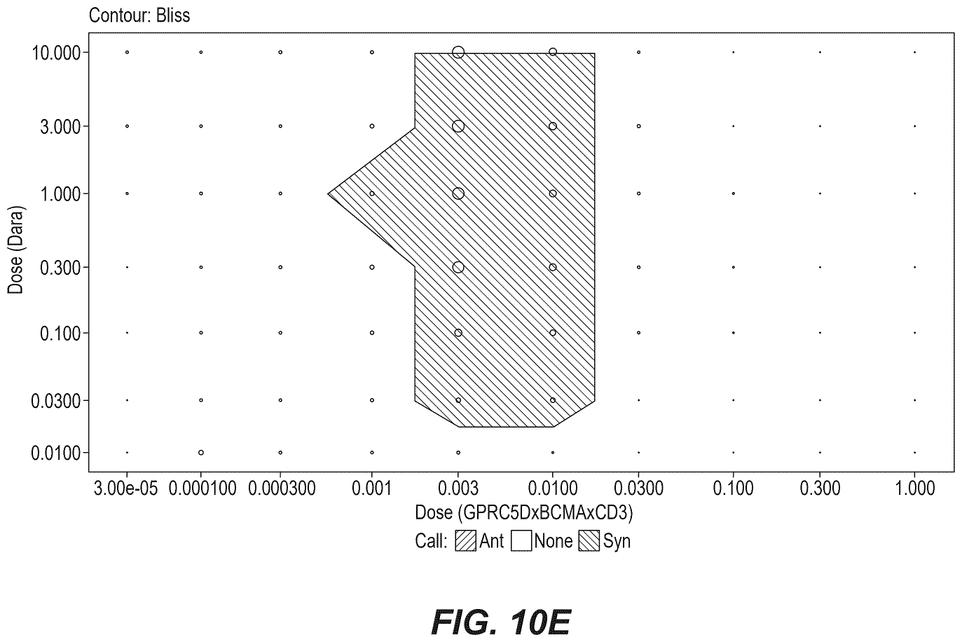

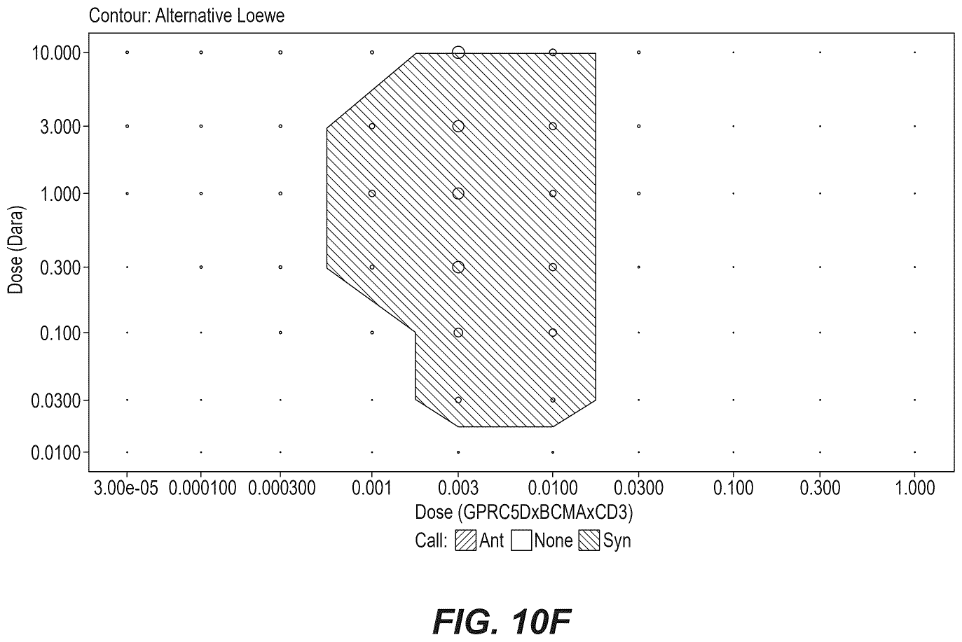

To determine the impact of daratumumab on BGCB491-mediated cytotoxicity, H929 cells were incubated for 48 hours with healthy donor derived PBMC in the presence of both compounds (n=3). A dose dependent increase in tumor cytotoxicity was observed with both BGCB491 and daratumumab monotherapy (FIG. 1A, FIG. 2A, AND FIG. 3A). The combination of daratumumab with BGCB491 resulted in decreased EC₅₀ values thus enhancing the potency compared to monotherapy of either BGCB491 or daratumumab. However, maximum tumor cytotoxicity of H929 cells did not change with daratumumab (FIGS. 1A-B, 2A-B, and 3A-B). To determine if the combination of daratumumab with BGCB491 was synergistic, synergy analysis on tumor cytotoxicity was performed as described above. Statistically significant synergy (p-value <0.05) in tumor cytotoxicity was observed using three independent models; HSA (FIG. 1C, FIG. 2C, and FIG. 3C), Bliss (FIG. 10A, FIG. 10C, and FIG. 10E) and alternative Loewe (FIG. 10B, FIG. 10D, and FIG. 10F). BGCB491 increases T cell activation measured as the percentage of CD25 positive or CD69 positive T cells in a concentration dependent manner (FIG. 4B and FIG. 4C, FIG. 5B and FIG. 5C, and FIG. 6B and FIG. 6C). Daratumumab did not impact T cell frequency (FIG. 4A, FIG. 5A, and FIG. 6A) or T cell activation as measured by the expression of CD25 (FIG. 4B, FIG. 5B, and FIG. 6B) and CD69 on T cells (FIG. 4C, FIG. 5C, and FIG. 6C) compared to BGCB491 monotherapy. Since daratumumab is an anti-CD38 antibody, CD38 expression was then evaluated to determine daratumumab binding to T and NK cells. PBMC donors 2 and 3 showed an increase in the levels of T cell CD38 MFI with increasing concentrations of BGCB491, which was decreased with the addition of Daratumumab (FIG. 5D and FIG. 6D) indicative of Daratumumab binding to CD38 on T cells. PBMC donor R100604 did not show a decrease in the levels of T cell CD38 MFI with the addition of daratumumab (FIG. 4D). Since one of the primary mechanisms of action for daratumumab is NK cell-mediated ADCC, NK cell activation was evaluated. Co-treatment of BGCB491 with daratumumab did not impact NK cells frequency compared to BGCB491 monotherapy (FIG. 7A, FIG. 8A, and FIG. 9A). A daratumumab-mediated dose-dependent increase in NK cell activation was observed as measured by the increase in the percentage of CD56+ CD25+ (FIG. 7B, FIG. 8B, and FIG. 9B) and CD56+ CD69+ (FIG. 7C, FIG. 8C, and FIG. 9c) NK cells. Increasing concentrations of BGCB491 resulted in increases in NK cell CD38 median fluorescence intensity (MFI) (donors 2 and 3), which was decreased with the addition of daratumumab (FIG. 8D and FIG. 9d). PBMC donor 1 did not show a decrease in the levels of NK cell CD38 MFI with the addition of daratumumab (FIG. 7D).

Overall, the results of in vitro immune killing assays with the co-treatment BGCB491 and daratumumab indicate: 1. BGCB491 in combination with daratumumab resulted in synergistic tumor cytotoxicity of H929 MM cells relative to both BGCB491; 2. daratumumab monotherapy treatments; and 3. Daratumumab does not impair BGCB491-mediated T cell activation, and combination of BGCB491 with daratumumab does not impair daratumumab-mediated NK cell activation.

### Example 3: A Phase 1B Study of BGCB491 in Combination with Daratumumab or Pomalidomide for Multiple Myeloma

This is an open-label, nonrandomized, multicenter, Phase 1b, dose-escalation study to evaluate the safety and tolerability, PK, pharmacodynamics, and preliminary antitumor activity of BGCB491 in combination with either daratumumab (Regimen A, and as of 13 Mar. 2025 Regimens C, D and E) or pomalidomide (Regimen B) and to identify the optimal dose(s) of the treatment regimen in participants with MM.

TABLE 4
Objectives and Endpoints

| | |
|---|---|
| Objectives | Endpoints |

Primary

| | |
|---|---|
| Part 1 (Dose Escalation) | Occurrence of DLTs |
| Determine RP2D(s) of BGCB491 in treatment | Occurrence and severity of AEs |
| regimens | and assessment of laboratory |
| | values |
| Part 2 (Dose Expansion) | Occurrence and severity of |
| Further characterize safety and tolerability of | AEs and assessment of |
| BGCB491 treatment regimens at the RP2D(s) and | laboratory values |
| in disease subgroups, as appropriate | |

Secondary

| | |
|---|---|
| Assess antitumor activity of the treatment regimens | Response as defined by IMWG |
| | 2016 response criteria |
| | DOR and TTR where response |
| | is defined by IMWG 2016 |
| | criteria |
| To assess the PK of BGCB491 (all regimens) and | Serum concentrations and PK |
| daratumumab (Regimen A) | parameters of BGCB491 and |
| As of 13 Mar. 2025, to assess the PK of | daratumumab |
| BGCB491 (all regimens) and daratumumab | |
| (Regimens C/D/E) | |
| To assess the immunogenicity of BGCB491 (all | Presence of anti-drug |
| regimens) and daratumumab (Regimen A) | antibodies to BGCB491 and |
| | daratumumab |

### Overall Design

This is an open-label, nonrandomized, multicenter, Phase 1b, dose-escalation and expansion study of BGCB491 combination treatment regimens in participants with multiple myeloma (MM).

This study will be conducted in 2 parts: Part 1 (Dose Escalation) to establish the RP2D(s) of the BGCB491 combination treatment regimen and Part 2 (Dose Expansion) at the RP2D(s) for the combination treatment regimens and in specified participant population(s). Additional combination treatment regimens may be added in future protocol amendments. Treatment regimens may run in parallel.
Treatment Regimen A (BGCB491+daratumumab) will be evaluated first in participants who have received 1-3 prior lines of therapy, including a proteasome inhibitor (PI) and an immunomodulatory drug (Treatment Regimen A1). Depending on emerging safety data from at least 6 participants in the first cohort, enrollment may proceed in participants with newly diagnosed multiple myeloma (NDMM), who have high-risk cytogenetics (Treatment Regimen A2).
Treatment Regimen B (BGCB491+pomalidomide) will be evaluated in participants who have received ≥1 prior line of therapy, including a PI and lenalidomide, and are lenalidomide refractory OR≥2 prior lines of therapy, including a PI and lenalidomide.
As of 13 Mar. 2025, for Regimens C, D, and E, participants with NDMM will be enrolled. As per the protocol, as of 8 Aug. 2025, for Regimen C, participants with NDMM, who have high-risk cytogenetics will be enrolled.

During the study, safety will be monitored at each dose escalation step and at regular intervals during cohort expansion. Dose escalation and de-escalation will be guided by the BOIN design as below. For dose levels of BGCB491, refer to Table 5. Initial treatment dose for BGCB491 will be 50 mg Q4W.

TABLE 5
Dose Levels of BGCB491

| Dose Levels | Treatment dose of BGCB491 |
|---|---|
| Dose Level 4 | 300 mg Q4W |
| Dose Level 3 | 200 mg Q4W |
| Dose Level 2 | 100 mg Q4W |
| Dose Level 1 | 50 mg Q4W |
| Dose Level −1 | 20 mg Q4W |

### Number of Participants

Approximately 20 participants per combination treatment regimen will be evaluated in Part 1 to establish the RP2D up to approximately 100 participants in Part 1. However, the actual sample size will depend on the number of cohorts explored during dose escalation. In Part 2, approximately 20 participants may be evaluated at each of the RP2D(s) and each disease subgroup in each combination treatment regimen up to approximately 80 participants in Part 2.

### Treatment Groups and Duration

### Description of Treatments

BGCB491 dosing (all treatment regimens): Starting dose level will be 50 mg BGCB491 SC Q4W with a 5 mg step-up dose given prior to the first full treatment dose for Treatment Regimens A and B. As of 13 Mar. 2025, starting dose level will be 100 mg BGCB491 SC Q4W with a 5 mg step-up dose given prior to the first full treatment dose for Treatment Regimens C, D and E.
Treatment Regimen A: Daratumumab will be administered SC at 1800 mg daratumumab/30000 units hyaluronidase Q4W.
Treatment Regimen B: Pomalidomide will be administered PO at 2 mg daily for the first 21 days of a 28-day cycle (beginning on Cycle 2 Day 1 as of 13 Mar. 2025). Dose may be increased up to 4 mg beginning at Cycle 3 at the investigator's discretion.

### As of 13 Mar. 2025

Treatment Regimen C: Daratumumab will be administered SC at 1800 mg daratumumab/30000 units hyaluronidase weekly during Cycles 1 and 2, biweekly during Cycles 3 to 6, and once (on Day 1) during each subsequent 28-day cycle.
Treatment Regimen D: Daratumumab will be administered SC at 1800 mg daratumumab/30000 units hyaluronidase Q4W. Lenalidomide 25 mg daily for 21 days/cycle will commence on Cycle 2 Day 1.
Treatment Regimen E: Daratumumab will be administered SC at 1800 mg daratumumab/30000 units hyaluronidase weekly during Cycles 1 and 2, biweekly during Cycles 3 to 6, and once (on Day 1) during each subsequent 28-day cycle. Lenalidomide 25 mg daily for 21 days/cycle will commence on Cycle 2 Day 1.

The study consists of a Screening Period (up to 28 days before first dose of study drug) and a Treatment Period, and a Posttreatment Follow-up Period (up to 16 weeks).

### Study Dosing Schedule/Regimen

TABLE 6
PART1 - DOSE ESCALATION

| Prior Treatment | Treatment Regimen | Additional notes |
|---|---|---|
| Newly diagnosed [No prior | Dara 1800 mg Q4W + | |
| treatment] | BGCB491 50 mg Q4W, | |
| | 5 mg step up BGCB491 | |
| Newly diagnosed [No prior | Dara 1800 mg Q4W + | |
| treatment] | BGCB491 100 mg Q4W, | |
| | 5 mg step up BGCB491 | |
| Newly diagnosed [No prior | As of 13 Mar. 2025: Dara | |
| treatment] | 1800 mg Q4W + BGCB491 | |
| | 200 mg Q4W, 5 mg step up | |
| | BGCB491 | |
| Newly diagnosed [No prior | Dara 1800 mg Q4W + | |
| treatment] | BGCB491 300 mg Q4W, | |
| | 5 mg step up BGCB491 | |
| Newly diagnosed [No prior | As of 13 Mar. 2025: Dara | |
| treatment] | 1800 mg Q4W + 25 mg Len | |
| | daily for 21 days (for cycle | |
| | 2 and subsequent cycles) + | |
| | BGCB491 100 mg Q4W, | |
| | 5 mg step up BGCB491 | |
| Newly diagnosed [No prior | As of 13 Mar. 2025: Dara | |
| treatment] | 1800 mg Q4W + 25 mg Len | |
| | daily for 21 days (for cycle | |
| | 2 and subsequent cycles) + | |
| | BGCB491 200 mg Q4W, | |
| | 5 mg step up BGCB491 | |
| Newly diagnosed [No prior | As of 13 Mar. 2025: Dara | |
| treatment] | 1800 mg Q4W + 25 mg Len | |
| | daily for 21 days (for cycle | |
| | 2 and subsequent cycles) + | |
| | BGCB491 300 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | BGCB491 100 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | BGCB491 200 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | BGCB491 300 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | 25 mg Len daily for 21 | |
| | days (for cycle 2 and | |
| | subsequent cycles) + | |
| | BGCB491 100 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | 25 mg Len daily for 21 | |
| | days (for cycle 2 and | |
| | subsequent cycles) + | |
| | BGCB491 200 mg Q4W, | |
| | 5 mg step up BGCB491 | |
| Newly diagnosed [No prior | As of 13 Mar. 2025: Dara | |
| treatment] | 1800 mg (QW for cycles 1 | |
| | and 2, Q2W for cycles 3-6, | |
| | Q4W for subsequent cycles) + | |
| | 25 mg Len daily for 21 | |
| | days (for cycle 2 and | |
| | subsequent cycles) + | |
| | BGCB491 300 mg Q4W, | |
| | 5 mg step up BGCB491 | |
| 1-3 Prior Lines | Dara 1800 mg Q4W + | |
| | BGCB491 50 mg Q4W, | |
| | 5 mg step up BGCB491 | |
| 1-3 Prior Lines | Dara 1800 mg Q4W + | |
| | BGCB491 100 mg Q4W, | |
| | 5 mg step up BGCB491 | |
| 1-3 Prior Lines | As of 13 Mar. 2025: Dara | |
| | 1800 mg Q4W + BGCB491 | |
| | 200 mg Q4W, 5 mg step up | |
| | BGCB491 | |
| 1-3 Prior Lines | Dara 1800 mg Q4W + | |
| | BGCB491 300 mg Q4W, | |
| | 5 mg step up BGCB491 | |
| ≥1 Prior Lines | Pomalidomide 2 mg daily | Pomalidomide dose may be |
| | for 21 days + BGCB491 50 | increased up to 4 mg |
| | mg Q4W, 5 mg step up | beginning at Cycle 3. |
| | BGCB491 | |
| ≥1 Prior Lines | Pomalidomide 2 mg daily | Pomalidomide dose may be |
| | for 21 days + BGCB491 100 | increased up to 4 mg |
| | mg Q4W, 5 mg step up | beginning at Cycle 3. |
| | BGCB491 | |
| ≥1 Prior Lines | As of 13 Mar. 2025: | Pomalidomide dose may be |
| | Pomalidomide 2 mg daily | increased up to 4 mg |
| | for 21 days + BGCB491 200 | beginning at Cycle 3. |
| | mg Q4W, 5 mg step up | |
| | BGCB491 | |
| ≥1 Prior Lines | Pomalidomide 2 mg daily | Pomalidomide dose may be |
| | for 21 days + BGCB491 300 | increased up to 4 mg |
| | mg Q4W, 5 mg step up | beginning at Cycle 3. |
| | BGCB491 | |

TABLE 7
Administration of BGCB491 + Daratumumab
Q4W Treatment (Treatment Regimen A)

| Study Drug | BGCB491 | Daratumumab |
|---|---|---|
| Study drug | Refer to the IPPI for BGCB491 and to the SmPC/USPI for | |
| instructions | daratumumab for sourcing, preparation, handling, storage, and | |
| | administration. The study treatment labels will contain | |
| | information to meet the applicable regulatory requirements. | |
| Starting dose/dose | Step-up: 5 mg | 1800 mg daratumumab/30000 |
| level(s) | Treatment dose: 50 mg | units hyaluronidase |
| | Subsequent dose escalation | |
| | will occur as described. | |
| Route of | SC administration | SC administration given by |
| administration | Administered at alternating | a manual push over |
| | anatomical locations on the | approximately 3-5 minutes. |
| | abdomen. Other locations | Doses will be administered |
| | may be acceptable based | at alternating locations on |
| | on investigator or medical | the abdomen. |
| | monitor discretion. | In case of significant |
| | | discomfort during manual |
| | | injection, a slower injection |
| | | speed may be used, or the |
| | | total dose may be given in |
| | | 2 separate locations. See |
| | | the site IPPI for guidance |
| | | regarding injection location |
| | | when >1 component of the |
| | | study treatment regimen is |
| | | administered SC within |
| | | 24 hours. |
| Dosing | Day −8 to Day −2: | Cycles (28-day cycle): |
| Schedule/Regimen | Step-up (5 mg) | Day 1: Full dose |
| | Cycles (28-day cycle): | |
| | Day 1: Treatment dose | |

| | |
|---|---|
| | The number of and schedule for step-up doses of BGCB491 |
| | may be modified. The schedule for treatment doses of |
| | BGCB491 may be modified based on emerging data (e.g. |
| | fixed duration, alternative dosing such as Q2W, weight-based |
| | dosing). |
| | When daratumumab and BGCB491 are to be administered on |
| | the same day, daratumumab will be administered first. |
| | The first treatment dose of BGCB491 will be administered |
| | 3 hours ± 30 minutes after daratumumab. |
| | Subsequent treatment doses of BGCB491 will be administered |
| | 1 hour ± 15 minutes after daratumumab SC. |
| | When more than one component of study treatment is |
| | administered SC on the same day, each SC component should |
| | be injected at different anatomical locations. |
| | For all components of study treatment that are administered |
| | SC, the anatomical area of administration must be recorded |
| | and the area observed for injection-site reaction(s). |
| | BGCB491 will not be administered during or within 2 hours |
| | from resolution of any sARR developing from |
| | daratumumab SC. To ensure safety, the sponsor may mandate |
| | longer intervals between administration of daratumumab SC |
| | and BGCB491. |
| | BGCB491 step-up and treatment doses MUST be delayed |
| | until CRS and/or ICANS associated with prior dose have fully |
| | resolved. |
| Study Windows | During the DLT Evaluation Period, BGCB491 and daratumumab |
| | administration, study assessments, and other study-related |
| | activities may be performed within ±1 day of the scheduled visit. |
| | After the conclusion of the DLT Evaluation Period, there must be |
| | at least 24 days and no more than 35 days between each |
| | administration of daratumumab SC and BGCB491. Study |
| | assessments may be performed up to 1 day before the scheduled |
| | treatment visit. |
| Vital Sign and | Monitor vital signs as indicated in the Schedule of Activities |
| Clinical Safety | and observe participants following daratumumab administration |
| Monitoring | as described. |
| | Criteria described must be met before the participant is |
| | discharged from the site. |

TABLE 8
Administration of BGCB491 + Daratumumab Label
Treatment as of 13 Mar. 2025 (Treatment Regimen C)

| Study Drug | BGCB491 | Daratumumab |
|---|---|---|
| Study drug | Refer to the IPPI for BGCB491 and to the SmPC/USPI for | |
| instructions | daratumumab for sourcing, preparation, handling, storage, and | |
| | administration. The study treatment labels will contain information | |
| | to meet the applicable regulatory requirements. | |
| Starting dose/dose | Step-up: 5 mg | 1800 mg daratumumab/30000 |
| level(s) | Treatment dose: 100 mg | units hyaluronidase |
| | Subsequent dose escalation will | |
| | occur as described. | |
| Route of | SC administration | SC administration given by a |
| administration | Administered at alternating | manual push over |
| | anatomical locations on the | approximately 3-5 minutes. |
| | abdomen. Other locations | Doses will be administered at |
| | may be acceptable based on | alternating locations on the |
| | investigator or medical | abdomen. |
| | monitor discretion. | In case of significant |
| | | discomfort during manual |
| | | injection, a slower injection |
| | | speed may be used, or the |
| | | total dose may be given in |
| | | 2 separate locations. See the |
| | | site IPPI for guidance |
| | | regarding injection location |
| | | when >1 component of the |
| | | study treatment regimen is |
| | | administered SC within |
| | | 24 hours. |
| Dosing | Day −8 to Day −2: | Cycles (28-day cycle): |
| Schedule/Regimen | Step-up (5 mg) | Cycles 1 and 2: Days 1, 8, 15 and |
| | Cycles (28-day cycle): | 22 (weekly) |
| | Day 1: Treatment dose | Cycles 3 to 6: Days 1 and 15 |
| | | (biweekly) |
| | | Cycles 7+: Day 1 |

| | |
|---|---|
| | The number of and schedule for step-up doses of BGCB491 may |
| | be modified by the SET. The schedule for treatment doses of |
| | BGCB491 may be modified by the SET based on emerging data |
| | (e.g. fixed duration, alternative dosing such as Q2W, weight- |
| | based dosing). |
| | When daratumumab and BGCB491 are to be administered on the |
| | same day, daratumumab will be administered first. |
| | The first treatment dose of BGCB491 will be administered |
| | 3 hours ± 30 minutes after daratumumab. |
| | Subsequent treatment doses of BGCB491 will be administered 1 |
| | hour ± 15 minutes after daratumumab SC. |
| | When more than one component of study treatment is |
| | administered SC on the same day, each SC component should be |
| | injected at different anatomical locations. |
| | For all components of study treatment that are administered SC, |
| | the anatomical area of administration must be recorded in the |
| | eCRF and the area observed for injection-site reaction(s). |
| | BGCB491 will not be administered during or within 2 hours from |
| | resolution of any sARR developing from daratumumab SC. To |
| | ensure safety, the sponsor may mandate longer intervals between |
| | administration of daratumumab SC and BGCB491. |
| | BGCB491 step-up and treatment doses MUST be delayed until |
| | CRS and/or ICANS associated with prior dose have fully |
| | resolved. |
| Study Windows | During the DLT Evaluation Period, BGCB491 and daratumumab |
| | administration, study assessments, and other study-related activities |
| | may be performed within ±1 day of the scheduled visit. |
| | After the conclusion of the DLT Evaluation Period, there must be at |
| | least 24 days and no more than 35 days between each administration |
| | of daratumumab SC and BGCB491. Study assessments may be |
| | performed up to 1 day before the scheduled treatment visit. |
| Vital Sign and | Monitor vital signs as indicated in the Schedule of Activities and |
| Clinical Safety | observe participants following daratumumab administration as |
| Monitoring | described. Criteria must be met before the participant is discharged |
| | from the site. |

TABLE 9
Administration of BGCB491 + Daratumumab Q4W + Lenalidomide
Treatment as of 13 Mar. 2025 (Treatment Regimen D)

| Study Drug | BGCB491 | Daratumumab | Lenalidomide |
|---|---|---|---|
| Study drug | Refer to the IPPI for BGCB491 and to the SmPC/USPI for | | |
| instructions | daratumumab and lenalidomide for sourcing, preparation, handling, | | |
| | storage, and administration. The study treatment labels will contain | | |
| | information to meet the applicable regulatory requirements. | | |
| Starting dose/dose | Step-up: 5 mg | 1800 mg daratumumab/ | 25 mg |
| level(s) | Treatment dose: | 30000 units | For participants with |
| | 100 mg | hyaluronidase | CrCl ≤60 mL/min. |
| | Subsequent dose | | |
| | escalation will | | |
| | occur as | | |
| | described. | | |
| Route of | SC | SC administration | Self-administered |
| administration | administration | given by a manual | orally. |
| | Administered | push over | |
| | at alternating | approximately 3- | |
| | anatomical | 5 minutes. | |
| | locations on | Doses will be | |
| | the abdomen. | administered at | |
| | Other | alternating locations | |
| | locations | on the abdomen. | |
| | may be | In case of | |
| | acceptable | significant | |
| | based on | discomfort during | |
| | investigator | manual injection, a | |
| | or medical | slower injection | |
| | monitor | speed may be used, | |
| | discretion. | or the total dose | |
| | | may be given in | |
| | | 2 separate locations. | |
| | | See the site IPPI for | |
| | | guidance regarding | |
| | | injection location | |
| | | when >1 component | |
| | | of the study | |
| | | treatment regimen | |
| | | is administered SC | |
| | | within 24 hours. | |
| Dosing | Day −8 to Day −2: | Cycles (28-day cycle): | Cycle 2+: Days 1 to |
| Schedule/Regimen | Step-up (5 mg) | Day 1: Full dose | 21, once daily. |
| | Cycles (28-day | | Per SET decision, |
| | cycle): | | subsequent |
| | Day 1: Treatment | | participants may |
| | dose | | receive lenalidomide |
| | | | 10 mg daily. |

| | |
|---|---|
| | The number of and schedule for step-up doses of BGCB491 may |
| | be modified by the SET. The schedule for treatment doses of |
| | BGCB491 may be modified by the SET based on emerging data |
| | (e.g. fixed duration, alternative dosing such as Q2W, weight- |
| | based dosing). |
| | The dose of lenalidomide may be adjusted by the SET. |
| | When daratumumab and BGCB491 are to be administered on |
| | the same day, daratumumab will be administered first. |
| | Beginning at Cycle 2 Day 1, when lenalidomide, daratumumab, |
| | and BGCB491 are to be administered on the same day, |
| | lenalidomide will be administered first. |
| | The first treatment dose of BGCB491 will be administered |
| | 3 hours ± 30 minutes after daratumumab. |
| | Subsequent treatment doses of BGCB491 will be administered 1 |
| | hour ± 15 minutes after daratumumab SC. |
| | When more than one component of study treatment is |
| | administered SC on the same day, each SC component should be |
| | injected at different anatomical locations. |
| | For all components of study treatment that are administered SC, |
| | the anatomical area of administration must be recorded in the |
| | eCRF and the area observed for injection-site reaction(s). |
| | BGCB491 will not be administered during or within 2 hours |
| | from resolution of any sARR developing from daratumumab SC. |
| | To ensure safety, the sponsor may mandate longer intervals |
| | between administration of daratumumab SC and BGCB491. |
| | BGCB491 step-up and treatment doses MUST be delayed until |
| | CRS and/or ICANS associated with prior dose have fully |
| | resolved. |
| | Dexamethasone will be given concomitantly with lenalidomide |
| | for Cycles 2 to 4. The dose is dexamethasone 40 mg weekly |
| | (Days 1, 8, 15, 22) from Cycle 2 Day 1 through the end of Cycle |
| | 4. For participants >75 years of age or ≤75 years of age and |
| | BMI <18.5, dexamethasone may be administered at a dose of 20 mg |
| | weekly. After the required dexamethasone cycles, the |
| | continuation of and the administration schedule for |
| | dexamethasone to enhance IMiD-driven antimyeloma effects |
| | will be based on the clinical judgement of the investigator. |
| Study Windows | During the DLT Evaluation Period, BGCB491 and daratumumab |
| | administration, study assessments, and other study-related activities |
| | may be performed within ±1 day of the scheduled visit. |
| | After the conclusion of the DLT Evaluation Period, there must be at |
| | least 24 days and no more than 35 days between each |
| | administration of daratumumab SC and BGCB491. Study |
| | assessments may be performed up to 1 day before the scheduled |
| | treatment visit. |
| Vital Sign and | Monitor vital signs as indicated in the Schedule of Activities and |
| Clinical Safety | observe participants following daratumumab administration as |
| Monitoring | described. Criteria must be met before the participant is discharged |
| | from the site. |

TABLE 10
Administration of BGCB491 + Daratumumab Label + Lenalidomide
Treatment as of 13 Mar. 2025 (Treatment Regimen E)

| Study Drug | BGCB491 | Daratumumab | Lenalidomide |
|---|---|---|---|
| Study drug | Refer to the IPPI for BGCB491 and to the SmPC/USPI for | | |
| instructions | daratumumab and lenalidomide for sourcing, preparation, handling, | | |
| | storage, and administration. The study treatment labels will contain | | |
| | information to meet the applicable regulatory requirements. | | |
| Starting dose/ | Step-up: 5 mg | 1800 mg daratumumab/ | 25 mg |
| dose level(s) | Treatment dose: | 30000 units | For participants |
| | 100 mg | hyaluronidase | with CrCl ≤60 |
| | Subsequent dose | | mL/min. |
| | escalation will | | |
| | occur as described. | | |
| Route of | SC | SC administration | Self-administered |
| administration | administration | given by a manual | orally. |
| | Administered at | push over | |
| | alternating | approximately 3- | |
| | anatomical | 5 minutes. | |
| | locations on the | Doses will be | |
| | abdomen. Other | administered at | |
| | locations may | alternating locations | |
| | be acceptable | on the abdomen. | |
| | based on | In case of significant | |
| | investigator or | discomfort during | |
| | medical monitor | manual injection, a | |
| | discretion. | slower injection | |
| | | speed may be used, | |
| | | or the total dose may | |
| | | be given in 2 separate | |
| | | locations. See the site | |
| | | IPPI for guidance | |
| | | regarding injection | |
| | | location when >1 | |
| | | component of the | |
| | | study treatment | |
| | | regimen is | |
| | | administered SC | |
| | | within 24 hours. | |
| Dosing | Day −8 to Day −2: | Cycles (28-day cycle): | Cycle 2+: Days 1 |
| Schedule/Regimen | Step-up (5 mg) | Cycles 1 and 2: Days 1, 8, | to 21, once daily. |
| | Cycles (28-day | 15 and 22 (weekly) | Per SET decision, |
| | cycle): | Cycles 3 to 6: Days 1 and | subsequent |
| | Day 1: Treatment | 15 (biweekly) | participants may |
| | dose | Cycles 7+: Day 1 | receive |
| | | | lenalidomide |
| | | | 10 mg daily. |

| | |
|---|---|
| | The number of and schedule for step-up doses of BGCB491 may |
| | be modified by the SET. The schedule for treatment doses of |
| | BGCB491 may be modified by the SET based on emerging data |
| | (e.g. fixed duration, alternative dosing such as Q2W, weight-based |
| | dosing). |
| | The dose of lenalidomide may be adjusted by the SET. |
| | When daratumumab and BGCB491 are to be administered on the |
| | same day, daratumumab will be administered first. |
| | Beginning at Cycle 2 Day 1, when lenalidomide, daratumumab, |
| | and BGCB491 are to be administered on the same day, |
| | lenalidomide will be administered first. |
| | The first treatment dose of BGCB491 will be administered 3 hours ± |
| | 30 minutes after daratumumab. |
| | Subsequent treatment doses of BGCB491 will be administered 1 |
| | hour ± 15 minutes after daratumumab SC. |
| | When more than one component of study treatment is administered |
| | SC on the same day, each SC component should be injected at |
| | different anatomical locations. |
| | For all components of study treatment that are administered SC, |
| | the anatomical area of administration must be recorded in the |
| | eCRF and the area observed for injection-site reaction(s). |
| | BGCB491 will not be administered during or within 2 hours from |
| | resolution of any sARR developing from daratumumab SC. To |
| | ensure safety, the sponsor may mandate longer intervals between |
| | administration of daratumumab SC and BGCB491. |
| | BGCB491 step-up and treatment doses MUST be delayed until |
| | CRS and/or ICANS associated with prior dose have fully resolved. |
| | Dexamethasone will be given concomitantly with lenalidomide for |
| | Cycles 2 to 4. The dose is dexamethasone 40 mg weekly (Days 1, |
| | 8, 15, 22) from Cycle 2 Day 1 through the end of Cycle 4. For |
| | participants >75 years of age or ≤75 years of age and BMI <18.5, |
| | dexamethasone may be administered at a dose of 20 mg weekly. |
| | After the required dexamethasone cycles, the continuation of and |
| | the administration schedule for dexamethasone to enhance IMiD- |
| | driven antimyeloma effects will be based on the clinical judgement |
| | of the investigator. |
| Study Windows | During the DLT Evaluation Period, BGCB491 and daratumumab |
| | administration, study assessments, and other study-related activities |
| | may be performed within ±1 day of the scheduled visit. |
| | After the conclusion of the DLT Evaluation Period, there must be at |
| | least 24 days and no more than 35 days between each administration |
| | of daratumumab SC and BGCB491. Study assessments may be |
| | performed up to 1 day before the scheduled treatment visit. |
| Vital Sign and | Monitor vital signs as indicated in the Schedule of Activitie and |
| Clinical Safety | observe participants following daratumumab administration as |
| Monitoring | described. |
| | Criteria must be met before the participant is discharged from the |
| | site. |

### Part 2—Dose Expansion

Dose expansion at RP2Ds and selected populations.

### Study Population

Screening for eligible participants will be performed within 28 days before administration of the study treatment.

The inclusion and exclusion criteria for enrolling participants in this study are described below. If there is a question about these criteria, the investigator must consult with the appropriate sponsor representative and resolve any issues before enrolling a participant in the study. Waivers are not allowed.

### Inclusion Criteria

Each potential participant must satisfy all of the following criteria to be enrolled in the study:

### Age

1. Be ≥18 years of age (or the legal age of majority in the jurisdiction in which the study is taking place, whichever is greater) at the time of informed consent.

### Type of Participant and Disease Characteristics

2. Have documented initial diagnosis of multiple myeloma according to IMWG diagnostic criteria.
3. Meet treatment regimen-specific requirements as follows:
Treatment Regimen A (BGCB491+daratumumab):
Treatment Regimen A1: Have been treated with 1 to 3 prior lines of therapy, including a PI and an IMiD therapy for the treatment of MM.
Treatment Regimen A2: Newly diagnosed MM naïve to multiple myeloma (or other related plasma cell neoplasm)-directed treatments that demonstrate high-risk cytogenetics (del[17p], t[4;14], t[14;16], or 1q21 gain/1q21 amplification). NOTE: At least 6 participants with 1 to 3 prior lines of therapy will be enrolled before SET may agree that participants with newly diagnosed disease can be enrolled.
Treatment Regimen B (BGCB491+pomalidomide): Have received ≥1 prior line of therapy, including a PI and lenalidomide, and are lenalidomide refractory OR≥2 prior lines of therapy, including a PI and lenalidomide.
As of 13 Mar. 2025, Treatment Regimens C, D, and E: Newly diagnosed MM naïve to multiple myeloma (or other related plasma cell neoplasm)-directed treatments. As per the protocol, as of 8 Aug. 2025, for Treatment Regimen C: Newly diagnosed MM naïve to multiple myeloma (or other related plasma cell neoplasm)-directed treatments that demonstrate high-risk cytogenetics (del[17p], t[4;14], t[14;16], or 1q21 gain/1q21 amplification).
4. Have measurable disease at screening as defined by at least 1 of the following:
Serum M-protein level ≥0.5 g/dL; or.
Urine M-protein level ≥200 mg/24 hours; or.
Light chain multiple myeloma: Serum Ig FLC≥10 mg/dL and abnormal serum Ig kappa lambda FLC ratio.
For participants without measurable disease in the serum, urine, or involved FLC: presence of 1 or more focus of EMD which meets the following criteria: extramedullary plasmacytoma not contiguous with a bone lesion, at least 1 lesion ≥2 cm (at its greatest dimension) diameter on whole body PET-CT (or whole-body MRI approved by sponsor), and not previously radiated.

### Weight

5. Have a weight ≥40 kg.

### Performance Status

6. Have an ECOG performance status of 0 to 2.
   Clinical Chemistry, including Renal and Hepatic Function
7. Have an eGFR of >30 ml/min/1.73m2 computed with the online calculator on the CKD-EPI website (http://ckdepi.org/equations/gfr-calculator/) as the CKD-EPIcr result.

As of 15 Nov. 2024 eGFR is calculated per 2021 CKD-epi creatinine equation (Inker L A, Eneanya N D, Coresh J, et al; Chronic Kidney Disease Epidemiology Collaboration. New Creatinine- and Cystatin C-Based Equations to Estimate GFR without Race. N Engl J Med. 2021; 385 (19): 1737-1749).
8. Have corrected serum calcium for albumin of ≤14 mg/dL (≤3.5 mmol/L) or free ionized calcium ≤6.5 mg/dL (≤1.6 mmol/L).
9. Participants are eligible if they have the following lab values:

$$\begin{matrix}AST \leq 3 \times ULN \\ ALT \leq 3 \times ULN\end{matrix}$$

Total bilirubin ≤1.5×ULN (isolated total bilirubin ≥1.5×ULN with conjugated [direct] bilirubin <ULN is allowed for those participants with known congenital nonhemolytic hyperbilirubinemia such as Gilbert's syndrome)

### Hematologic Values

10. Participants are eligible if they have the following lab values:
Hemoglobin ≥8.0 g/dL (≥5 mmol/L) (without prior RBC transfusion within 7 days prior to the laboratory test; recombinant human erythropoietin use is permitted)
Neutrophils ≥1.0×10⁹/L (without use of G-CSF or GM-CSF within the 7 days prior to the date of the laboratory test or 14 days for pegylated G-CSF)
Platelets ≥50×109/L (without transfusion support in the 7 days prior to the laboratory test)

### Sex and Contraceptive/Barrier Requirements

11. While on study treatment and for 6 months after the last dose of study treatment (unless otherwise noted below), a participant must:
Not breastfeed or be pregnant.
Not donate gametes (ie, eggs [minimum of 6 months] or sperm [minimum of 3 months]) or freeze for future use for the purposes of assisted reproduction. Participants should consider preservation of gametes (ie, eggs or sperm) prior to study treatment as anticancer treatments may impair fertility.
Must wear a condom when engaging in any activity that allows for passage of ejaculate to another person during the study and for 3 months after receiving the last dose of study treatment.
If of childbearing potential,
have a negative highly sensitive (eg, beta-human chorionic gonadotropin [β-hCG]) pregnancy test at screening and within 72 hours (as of 13 Mar. 2025: within 2 days) before the first dose of study treatment, and agree to further pregnancy tests while on study,
Regimen A (as of 13 Mar. 2025 also Regimen C): practice at least 1 highly effective method of contraception; if oral contraceptives are used, a barrier method of contraception must also be used
OR
Regimen B (as of 13 Mar. 2025 also Regimens D and E); 2 methods of reliable birth control simultaneously, including 1 highly effective form of contraception and 1 additional effective contraceptive method.
   The investigator should evaluate the potential for contraceptive method failure (eg, noncompliance, recently initiated) in relationship to the first dose of study drug.
   Note: If a participant becomes of childbearing potential after the start of the study, the participant must comply with this bullet point.
If a participant's partner is of childbearing potential,
the participant must use condom (with or without spermicide) and the partner must also be practicing a highly effective method of contraception. A participant who is vasectomized must still use a condom (with or without spermicide), but the partner is not required to use contraception.

### Informed Consent

12. Must sign an ICF indicating that the participant understands the purpose of, and procedures required for, the study and is willing to participate in the study.
13. Be willing and able to adhere to the lifestyle restrictions specified in this protocol, including adherence to the applicable global IMID PPP or local PPP/REMS program for participants receiving Treatment Regimen B.

### Exclusion Criteria

Any potential participant who meets any of the following criteria will be excluded from participating in the study:

### Medical Conditions

1. Pulmonary compromise requiring supplemental oxygen.
2. Any serious underlying medical conditions, such as:
Evidence of active viral, bacterial, or systemic fungal infection requiring ongoing antiviral, antibacterial, or antifungal treatment.
Active autoimmune disease requiring systemic immunosuppressive therapy within 6 months before start of study treatment. EXCEPTION: Participants with vitiligo, type I diabetes, and prior autoimmune thyroiditis that is currently euthyroid based on clinical symptoms and laboratory testing are eligible regardless of when these conditions were diagnosed.
Disabling psychiatric conditions, substance abuse (eg, alcohol or drug abuse), severe dementia, or altered mental status.
Any other issue that would impair the ability of the participant to receive or tolerate the planned treatment at the investigational site, to understand the informed consent, or any condition for which, in the opinion of the investigator, participation would not be in the best interest of the participant (eg, compromise the well-being of the participant) or that could prevent, limit, or confound the protocol-specified assessments.
3. Have a prior or concurrent second malignancy (other than the disease under study) which natural history or treatment is likely to interfere with any study endpoints of safety or the efficacy of the study treatment(s).
4. History of stroke or seizure within 6 months prior to the first dose of study treatment.
5. Known allergies, hypersensitivity, or intolerance to any study treatment or their excipients (refer to the applicable IB or SmPC/USPI).
6. Had major surgery or had significant traumatic injury within 2 weeks, or not fully recovered from surgery, or participant has major surgery planned during the time he or she is expected to participate in the study.

### Cardiovascular Dysfunction

7. Participant has a history of clinically significant cardiovascular disease including, but not limited to the following:
New York Heart Association Stage III or IV congestive heart failure.
Myocardial infarction, unstable angina, or coronary artery bypass graft ≤6 months prior to enrollment.
History of clinically significant ventricular arrhythmia or unexplained syncope not believed to be vasovagal in nature or due to dehydration.
History of severe nonischemic cardiomyopathy.

### Disease Characteristics

8. Central nervous system involvement or clinical signs of meningeal involvement of multiple myeloma. If either is suspected, whole brain MRI and lumbar cytology are required during screening
9. Active plasma cell leukemia, Waldenström's macroglobulinemia, POEMS syndrome (polyneuropathy, organomegaly, endocrinopathy, M-protein, and skin changes), or primary light chain amyloidosis.

### HIV Status

10. Known history of HIV infection.

### Viral Hepatitis Assessments

11. Active hepatitis of infectious origin.
a. Seropositive for hepatitis B: defined by a positive test for HBsAg. Participants with resolved infection (ie, participants who are HBsAg negative with positive antibodies to total anti-HBc with or without the presence of antiHBs) must be screened using RT-PCR measurement of HBV DNA levels. Those who are RT-PCR positive, even those participants with results below the detection limit, will be excluded. Those participants who have a resolved infection and test negative by RT-PCR at screening should be retested using RT-PCR every 3 months. Participants with serologic findings suggestive of HBV vaccination (anti-HBs positivity as the only serologic marker) AND a known history of prior HBV vaccination, do not need to be tested for HBV DNA by RT-PCR. As of 13 Mar. 2025 a known history of prior HBV vaccination no longer required.
b. Known hepatitis C infection or positive serologic testing for hepatitis C virus (anti-HCV) antibody.
c. Participants with positive hepatitis C antibody due to prior resolved disease can be enrolled only if a confirmatory negative hepatitis C RNA test is obtained at screening or within 3 months prior to first dose of study treatment.
d. Other clinically active liver disease of infectious origin.

### Prior/Concomitant Therapy

12. Prior treatment with CD3-redirecting therapy.
13. Prior antitumor therapy as follows, in the specified time frame prior to the first dose of study treatment:
Targeted therapy, epigenetic therapy, mAb treatment, or treatment with an investigational drug or an invasive investigational medical device within 21 days or 5 half-lives, whichever is less.
Gene-modified adoptive cell therapy (eg, CAR modified T cells, natural killer cells) within 90 days.
Prior anti-CD38 directed therapy within 90 days. (for Treatment Regimen A only; within 21 days for Treatment Regimen B).
Conventional chemotherapy within 21 days.
PI therapy within 14 days.
Immunomodulatory agent therapy within 7 days.
Radiotherapy within 14 days. However, if palliative focal radiation was used, the participant is eligible irrespective of the end date of radiotherapy.
14. Received a cumulative dose of corticosteroids equivalent to >140 mg of prednisone within the 14-day period before the first dose of study treatment (does not include pretreatment medications).
15. Stem cell transplantation:
Allogeneic stem cell transplant within 6 months before the first dose of study treatment. Participants who received an allogeneic transplant must be off all immunosuppressive medications for ≥42 days without signs of graft-versus-host disease.
Received an autologous stem cell transplant ≤12 weeks before the first dose of study treatment.
16. Nonhematologic toxicity from prior anticancer therapy that has not resolved to baseline level or to Grade ≤1 (except alopecia, tissue post-RT fibrosis [any grade] or peripheral neuropathy Grade ≤3).
17. Received or plans to receive any live, attenuated vaccine within 4 weeks before the first dose of study drug.

### Other Exclusions

18. Treatment Regimen A only (as of 13 Mar. 2025 also Regimens C, D, and E): Participant has either of the following:
COPD with FEV1<50% of predicted normal. Note that FEV1 testing is required for participants suspected of having COPD, and participants must be excluded if FEV1 is <50% of predicted normal; testing done as standard of care within 6 months of the first dose of study treatment is acceptable for this criterion.
Moderate or severe persistent asthma within the past 2 years, or uncontrolled asthma of any classification. Note: participants who currently have controlled intermittent asthma or controlled mild persistent asthma are allowed to participate in the study.
19. Any condition for which, in the opinion of the investigator, participation would not be in the best interest of the participant (eg, compromise the well-being) or that could prevent, limit, or confound the protocol-specified assessments.
NOTE: Investigators must ensure that all study enrollment criteria have been met at screening. If a participant's clinical status changes (including any available laboratory results or receipt of additional medical records) after screening but before the first dose of study treatment is given such that the participant no longer meets all eligibility criteria, then the participant must be excluded from participation in the study.

### Lifestyle Considerations

Potential participants must be willing and able to adhere to the following lifestyle restrictions during the study to be eligible for participation:
1. Agree to self-monitor for signs and symptoms of CRS (such as fever) and to seek immediate medical intervention.
2. Any blood donation is not allowed until ≥90 days after the last dose of study treatment.
3. Refer to Section 6.9.7 for details regarding prohibited and restricted therapy during the study.
4. Agree to follow all requirements that must be met during the study as noted in the Inclusion and Exclusion Criteria (eg, contraceptive requirements).
5. Be willing to be hospitalized or remain at or in close proximity (within 30 mins travel time) to the study site for observation.
6. Carry a “wallet study card” with pertinent information about the study for the duration of study participation.
7. Treatment Regimen B only (as of 13 Mar. 2025 also Regimens D and E): Due to the embryofetal risk associated with pomalidomide, participants must adhere to the applicable pomalidomide global PPP or local PPP/REMS program.

### Efficacy Evaluations

Efficacy assessments will be evaluated per the IMWG 2016 criteria.

As of 16 May 2025, for 7 patients treated with 50 mg Tri Q4W+Dara Q4W, with approximately 2 months of minimum follow-up, the results are as follows:

2 patients are in partial response,

4 patients have stable disease, as defined by the IMWG criteria, which states that stable disease does not meet the criteria for complete response, very good partial response, partial response, minimal response, or progressive disease, 1 patient is not evaluable.

### Pharmacokinetic and Immunogenicity Evaluations

Blood samples will be assessed for serum concentrations of BGCB491 and daratumumab for pharmacokinetic analyses. The detection and characterization of antibodies to BGCB491 and daratumumab will be performed using validated assay methods to enable interpretation of the antidrug antibody data.

### Pharmacodynamic and Biomarker Evaluations

Baseline bone marrow aspirate samples will be used to characterize the clonal plasma cells and their immune microenvironment and may identify predictive biomarkers of response by gene expression profiling. Bone marrow and whole blood will be evaluated for immune infiltration and markers of immune-cell activation and/or exhaustion as well as BCMA and GPRC5D expression and immune checkpoint ligands. Using next-generation sequencing, bone marrow aspirate will be assessed for minimal residual disease.

### Safety Evaluations

Safety will be assessed by physical examinations, Eastern Cooperative Oncology Group performance status score, clinical laboratory tests, vital signs, and adverse event monitoring.

### Safety Data Summary as of 28 Apr. 2025

As of 3 Apr. 2025, 7 participants with relapsed/refractory multiple myeloma have been treated with BGCB491 50 mg Q4W (preceded by a step-up dose of 5 mg)+daratumumab 1800 mg Q4W. Treatment is administered in 28-day cycles. Median age was 67.0 years (range: 34 to 80), with 4 participants who were at least 65 years of age. 6 participants were male. The median number of prior lines of therapy was 2.0 (range: 1 to 3) and median time since initial diagnosis to first dose was 5.48 years (range: 2.0 to 11.1 years). Median follow-up was 1.91 months (range: 1.1 to 2.8 months).

All 7 participants experienced ≥1 TEAE. A maximum of Grade 3 TEAEs was reported for 1 participant and a maximum of Grade 4 TEAEs was reported for 1 participant. Grade 3 events included neutropenia, thrombocytopenia and headache; Grade 4 events included neutropenia. No participants reported Grade 5 events. Two participants experienced a serious TEAE (CRS and arthralgia in 1 participant, headache in 1 participant). No participants experienced a TEAE leading to discontinuation of BGCB491. No participants have experienced a DLT.

CRS was the most commonly reported TEAE (4 participants), with Grade 1 CRS experienced by 3 participants and Grade 2 CRS by 1 participant. No participants reported Grade 3 or higher CRS. All 4 CRS events have resolved. No ICANS was reported. Infections occurred in 1 participant (Grade 1 hordeolum and Grade 2 upper respiratory tract infection) with no Grade 3 or higher infections. The only other AE observed in ≥2 participants was arthralgia (2 participants).

Based on the preliminary data summarized above, it is considered that the BGCB491+daratumumab combination regimen appears to be safe and tolerable in participants with relapsed/refractory multiple myeloma.

Treatment response was assessed according to the IMWG 2016 criteria. These criteria are summarised in Table 11 below:

TABLE 11
International Myeloma Working Group 2016 Response Criteria

| Response | Response Criteria |
|---|---|
| sCR | CR as defined below, plus |
| | Normal FLC ratio, and |
| | Absence of clonal PCs by immunohistochemistry (κ/λ) ratio ≤4:1 |
| | or ≥1:2 for κ and λ participants, respectively, after counting ≥100 |
| | plasma cells) or negative 2-4 color flow cytometry |
| CR | Negative immunofixation of serum and urine, and |
| | Disappearance of any soft-tissue plasmacytomas, and |
| | <5% PCs in bone marrow |
| | No evidence of initial monoclonal protein isotype(s) on |
| | immunofixation of the serum and urine^{b} |
| VGPR | Serum and urine M-component detectable by immunofixation but |
| | not on electrophoresis, or |
| | ≥90% reduction in serum M-component plus urine |
| | M-component <100 mg/24 hours |
| | In addition to the above criteria, if present at baseline >90% |
| | reduction in the SPD compared with baseline for soft-tissue |
| | plasmacytoma |
| PR | ≥50% reduction of serum M-protein and reduction in 24-hour |
| | urinary M-protein by ≥90% or to <200 mg/24 hours |
| | If serum and urine M-protein are not measurable, a decrease ≥50% |
| | in the difference between involved and uninvolved FLC levels is |
| | required in place of the M-protein criteria |
| | In addition to the above criteria, if present at baseline, ≥50% |
| | reduction in the size (SPD) of soft-tissue plasmacytomas is also |
| | required |
| MR | ≥25% but ≤49% reduction of serum M-protein and reduction in 24- |
| | hour urine M-protein by 50% to 89% |
| | In addition to the above criteria, if present at baseline, ≥50% |
| | reduction in the size of soft-tissue plasmacytomas is also required |
| Stable | Not meeting criteria for sCR, CR, VGPR, PR, MR, or progressive |
| disease | disease |
| Progressive | Any 1 or more of the following criteria: |
| disease | Increase of 25% from lowest response value in any of the following: |
| | Serum M-component (absolute increase must be ≥0.5 g/dL), |
| | and/or |
| | Urine M-component (absolute increase must be ≥200 mg/24 |
| | hours), and/or |
| | Only in participants without measurable serum and urine M- |
| | protein levels: the difference between involved and uninvolved |
| | FLC levels (absolute increase must be >10 mg/dL) |
| | Appearance of a new lesion(s), ≥50% increase from nadir in SPD |
| | of >1 lesion, or ≥50% increase in the longest diameter of a previous |
| | lesion >1 cm in short axis |
| | Definite development of new bone lesions or definite increase in the |
| | size of existing bone lesions |
| | ≥50% increase in circulating PCs (minimum of 200 cells/μL) if this |
| | is the only measure of disease |

CR = complete response; FLC = free light chain; Ig = immunoglobulin; MR = minimal response; PC = plasma cell; PR = partial response; sCR = stringent complete response; SPD = sum of the products of the maximal perpendicular diameters of measured lesions; VGPR = very good partial response.

### Data Summary August-September 2025

As of 22 Aug. 2025, 84 participants with relapsed/refractory multiple myeloma have been treated with BGCB491 across the following treatment regimens:
Regimen A1: 19 patients
   7 patients were treated with 50 mg Q4W BGCB491 (preceded by a step-up dose of 5 mg)+daratumumab 1800 mg Q4W.
   12 patients were treated with 100 mg Q4W BGCB491 (preceded by a step-up dose of 5 mg)+daratumumab 1800 mg Q4W.
   The median follow-up time was 4.63 months.
Regimen A2: 26 patients
   6 patients were treated with 50 mg Q4W BGCB491 (preceded by a step-up dose of 5 mg)+daratumumab 1800 mg Q4W.
   20 patients were treated with 100 mg Q4W BGCB491 (preceded by a step-up dose of 5 mg)+daratumumab 1800 mg Q4W.
   The median follow-up time was 2.09 months.
Regimen B: 32 patients
   6 patients were treated with 50 mg Q4W BGCB491 (preceded by a step-up dose of 5 mg)+pomalidomide 2 mg daily for 21 days beginning Day 1 Cycle 2.
   20 patients were treated with 100 mg Q4W BGCB491 (preceded by a step-up dose of 5 mg)+pomalidomide 2 mg daily for 21 days beginning Day 1 Cycle 2.
   6 patients were treated with 200 mg Q4W BGCB491 (preceded by a step-up dose of 5 mg)+pomalidomide 2 mg daily for 21 days beginning Day 1 Cycle 2.
   The median follow-up time was 3.3 months.
Regimen C: 7 patients
   7 patients were treated with 100 mg Q4W BGCB491 (preceded by a step-up dose of 5 mg)+daratumumab weekly during Cycles 1 and 2, biweekly during Cycles 3 to 6, and once (on Day 1) during each subsequent 28-day cycle.
   The median follow-up time was 0.33 months.
      Treatment is administered in 28-day cycles. Patients ranged in age from 33 to 82 years and included both males and females, as well as Hispanic or Latino and non-Hispanic or Latino populations.

### Safety and Tolerability Data Summary as of 22 Aug. 2025

### As of 22 Aug. 2025:

Treatment-emergent adverse events (TEAEs) occurred in the majority of patients across all regimens.
Dose-limiting toxicities (DLTs) were reported in 2 patients (16.7%) at 100 mg in Regimen A1 and in 1 patient (5.0%) at 100 mg in Regimen B.
No patients discontinued study drug due to TEAEs.
Cytokine release syndrome (CRS) was the most frequent adverse event, observed in 14.3% to 57.9% of patients depending on regimen.
No cases of immune effector cell-associated neurotoxicity syndrome (ICANS) were reported.
Serious adverse events (SAEs) occurred in 37.5% to 47.4% of patients in Regimens A1, A2, and B, but none in Regimen C.
No deaths were reported as of 22 Aug. 2025.

TABLE 12
Safety Outcomes of BGCB491 Combinations (22 Aug. 2025)

| | | Any | DLTs | TEAE leading to | | | | |
|---|---|---|---|---|---|---|---|---|
| Regimen | N | TEAE | (%) | discontinuation | CRS | ICANS | SAEs | Deaths |
| A1 (1-3 prior lines, | | | | | | | | |
| Dara Q4W) | | | | | | | | |
| BGCB491: 50 mg | 7 | 7 | 0 | 0 | 4 | 0 | 2 | 0 |
| BGCB491: 100 mg | 12 | 12 | 2 | 0 | 7 | 0 | 7 | 0 |
| Total A1 | 19 | 19 | 2 | 0 | 11 | 0 | 9 | 0 |
| A2 (Newly diagnosed, | | | | | | | | |
| Dara Q4W) | | | | | | | | |
| BGCB491: 50 mg | 6 | 6 | 0 | 0 | 3 | 0 | 2 | 0 |
| BGCB491: 100 mg | 20 | 20 | 0 | 0 | 9 | 0 | 8 | 0 |
| Total A2 | 26 | 26 | 0 | 0 | 12 | 0 | 10 | 0 |
| B (≥1 prior line, | | | | | | | | |
| Pomalidomide) | | | | | | | | |
| BGCB491: 50 mg | 6 | 6 | 0 | 0 | 3 | 0 | 5 | 0 |
| BGCB491: 100 mg | 20 | 20 | 1 | 0 | 6 | 0 | 6 | 0 |
| BGCB491: 200 mg | 6 | 5 | 0 | 0 | 1 | 0 | 1 | 0 |
| Total B | 32 | 31 | 1 | 0 | 10 | 0 | 12 | 0 |
| C (Newly diagnosed, | | | | | | | | |
| Dara label schedule) | | | | | | | | |
| BGCB491: 100 mg | 7 | 5 | 0 | 0 | 1 | 0 | 0 | 0 |

### Efficacy Data Summary as of 9 Sep. 2025

Efficacy was evaluated using the International Myeloma Working Group (IMWG) 2016 response criteria. Objective responses were observed across regimens and dose levels, including stringent complete responses (sCR), very good partial responses (VGPR), partial responses (PR), stable disease (SD), progressive disease (PD), and not evaluable (NE). Results are summarized in Table 13.

TABLE 13
Efficacy Outcomes of BGCB491 Combinations (9 Sep. 2025, IMWG 2016)

| | | | | CR/ | | | Unconfirmed | | | |
|---|---|---|---|---|---|---|---|---|---|---|
| Regimen | Dose | | N | sCR | VGPR | PR | Responses | SD | PD | NE |
| Al (1-3 prior lines, | 50 | mg | 7 | — | 1 | 3 | — | 3 | — | — |
| Dara Q4W) | | | | | | | | | | |
| | 100 | mg | 12 | 1 | 6 | 2 | 1 PR | 1 | 1 | — |
| A2 (Newly diagnosed, | 50 | mg | 6 | — | 3 | 2 | 1 VGPR | — | — | — |
| Dara Q4W) | | | | | | | | | | |
| | 100 | mg | 20 | — | 4 | 5 | 5 PR | 5 | — | 1 |
| B (≥1 prior line, | 50 | mg | 6 | 1 sCR | 4 | 1 | — | — | — | — |
| Pomalidomide) | | | | | | | | | | |
| | 100 | mg | 20 | 1 sCR | 8 | 4 | — | 6 | 1 | — |
| | 200 | mg | 6 | — | 1 | — | 1 PR | 2 | — | 2 |
| C (Newly diagnosed, | 100 | mg | 7 | — | — | — | — | — | — | — |
| Dara label schedule) | | | | | | | | | | |

The clinical data demonstrate that BGCB491 can be safely combined with daratumumab or pomalidomide across multiple dose levels and patient populations, including both relapsed/refractory and newly diagnosed myeloma. Objective responses, including complete/stringent complete responses, very good partial responses, and partial responses, were observed. Notably, no ICANS events or treatment-related deaths were reported.

These results provide support for the therapeutic use of BGCB491 in combination with daratumumab or pomalidomide in the treatment of multiple myeloma, and demonstrate efficacy and tolerability across diverse regimens and patient populations.

### Example 4. Nonclinical Efficacy Study of BGCB491 and Lenalidomide Combination Treatment

### Study Design and Treatment

A summary of the study design is provided in Table 14. Day 0 was the day of tumor cell implantation and study initiation. T-cell-humanized mice were given fragment crystallizable (Fc) block at 0.2 mg/mouse IP and human immune globulin infusion at 10 mg/mouse IP at least 30 minutes prior to antibody dosing, to compensate for the low Ig environment in the immune-deficient NSG mouse.

TABLE 14
Study Design

| | | Mean tumor volume | | |
|---|---|---|---|---|
| | | at randomization | Day of T-cell | |
| Tumor model | Study Type | (Day) | engraftment | Treatment Days |
| RPMI 8226 | Efficacy | 94 mm³ (21) | 21 | DPBS/BGCB491- |
| | | | | 22, 25, 29, 32, 36, |
| | | | | 39, 43, 46 |
| | | | | vehicle/lenalidomide - |
| | | | | 22-49 (28 days) |

DPBS, Dulbecco's phosphate-buffered saline; RPMI, Roswell Park Memorial Institute.
The day of tumor cell implantation was Day 0.

Mice were injected SC with 1×10⁷ RPMI 8226 cells in the right flank. On Day 21 post tumor implantation, mice were randomized into groups of 10 animals each, according to tumor volume such that all groups had mean values of 94 mm³. Mice were humanized with T cells on the day of randomization, and treatment was initiated on Day 22, 1 day after T-cell implantation. BGCB491 at 0.1 or 0.4 mg/kg, or DPBS was administered IP twice a week for a total of 8 doses. Lenalidomide at 10 or 50 mg/kg, or vehicle was dosed PO daily on Days 22 to 49, for a total of 28 doses.

### Efficacy Results

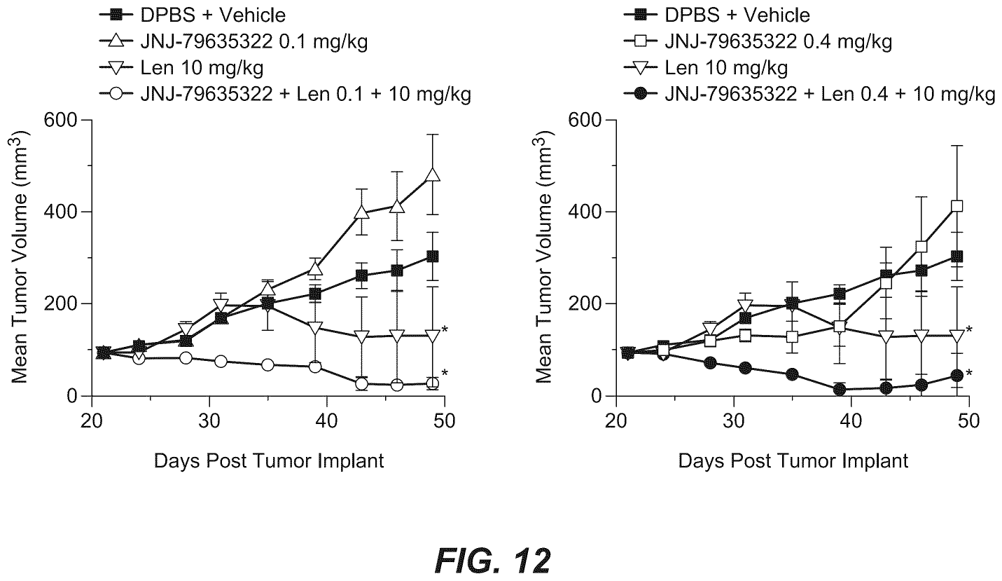

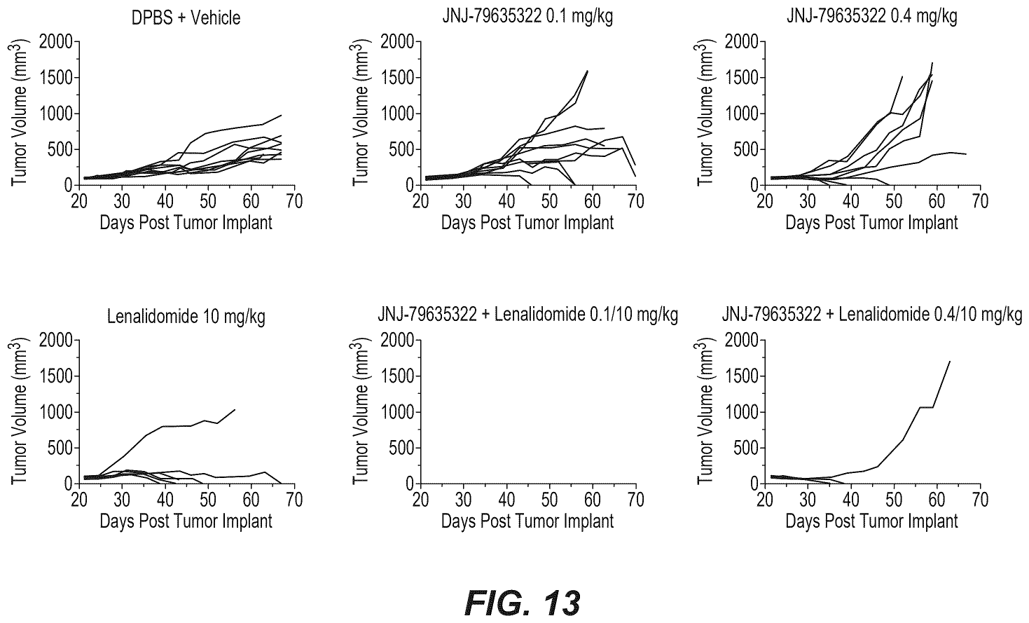

In T-cell-humanized mice bearing established RPMI 8226 xenografts treatment with monotherapy BGCB491 at both 0.1 and 0.4 mg/kg did not result in significant antitumor activity, and TGI was not observed as compared to DPBS+vehicle-treated control (see FIG. 2, Table 12), although 3 of 10 and 4 of 10 CRs were observed by the end of the study in each group, respectively (see FIG. 3). Significant antitumor efficacy was observed with lenalidomide single agent treatment at 10 mg/kg, as assessed by the change in mean tumor burden on Day 49 post tumor implantation (the last day of lenalidomide treatment), as compared to DPBS+vehicle-treated control group (p≤0.05), with 84% ΔTGI (see FIG. 12, Table 15), and 5 of 10 CRs (see FIG. 13). Individual tumor growth curves were plotted up to Day 67 and measurements collected on Day 71 were censored as GvHD was observed throughout each of the remaining groups (see FIG. 3). The combination treatment of BGCB491 at 0.1 mg/kg+lenalidomide at 10 mg/kg elicited significant antitumor efficacy as assessed by change in mean tumor burden (p≤0.05), resulting in 132% ΔTGI, as compared to the DPBS+vehicle control on Day 49 post tumor implantation (see FIG. 12 left panel, Table 15). The combination treatment of BGCB491 at 0.4 mg/kg+lenalidomide at 10 mg/kg elicited similarly significant antitumor efficacy as assessed by change in mean tumor burden (p≤0.05), with 123% ΔTGI, as compared to the DPBS+vehicle control on Day 49 post tumor implantation (see FIG. 12 left panel, Table 15). Although combination treatment was not significant compared to monotherapy, a trend of enhanced efficacy was observed with 8 of 10 and 9 of 10 CRs in combinations with BGCB491 at 0.1 and 0.4 mg/kg, respectively (see FIG. 13).

Treatment with lenalidomide single agent at 50 mg/kg demonstrated robust antitumor activity and similar trends of enhanced efficacy were observed when administered in combination with BGCB491 at both 0.1 and 0.4 mg/kg (data not shown).

TABLE 15
Summary of Results for BGCB491.

| | | | | Animals | |
|---|---|---|---|---|---|
| | | Species/ | | per | |
| Study | | Test | Treatment | Group | |
| (Reference) | Type | System | Duration | (F) | Dose Groups and Key Results |
| RPMI 8226 | SC | NSG mice/ | 4 weeks | 10 (F) | 0% ΔTGI at 0.1 mg/kg BGCB491(ns), 3/10 |
| Combination | Efficacy | T cells | | | CRs (Day 67), 0% ΔTGI at 0.4 mg/kg |
| | | | | | BGCB491(ns), 4/10 CRs (Day 67), Day 49 |
| | | | | | for ΔTGI; q3d-q4d IP for 8 doses |
| | | | | | Combination BGCB4910.1 mg/kg + |
| | | | | | lenalidomide 10 mg/kg: 132% ΔTGI, 8/10 |
| | | | | | CRs (Day 67), Day 49 for ΔTGI; |
| | | | | | BGCB491dosed q3d-q4d IP for 8 doses, |
| | | | | | lenalidomide dosed Days 22-49 PO for 28 |
| | | | | | doses |
| | | | | | Combination BGCB4910.4 mg/kg + |
| | | | | | lenalidomide 10 mg/kg: 123% ΔTGI, 9/10 |
| | | | | | CRs (Day 67), Day 49 for ΔTGI; |
| | | | | | BGCB491dosed q3d-q4d IP for 8 doses, |
| | | | | | lenalidomide dosed Days 22-49 PO for 28 |
| | | | | | doses |
| | | | | | Combination BGCB4910.1 mg/kg + |
| | | | | | lenalidomide 50 mg/kg: 145% ΔTGI, 9/10 |
| | | | | | CRs (Day 67), Day 49 for ΔTGI; |
| | | | | | BGCB491dosed q3d-q4d IP for 8 doses, |
| | | | | | lenalidomide dosed Days 22-49 PO for 28 |
| | | | | | doses |
| | | | | | Combination BGCB4910.4 mg/kg + |
| | | | | | lenalidomide 50 mg/kg: 145% ΔTGI, 10/10 |
| | | | | | CRs (Day 67), Day 49 for ΔTGI; |
| | | | | | BGCB491dosed q3d-q4d IP for 8 doses, |
| | | | | | lenalidomide dosed Days 22-49 PO for 28 |
| | | | | | doses |

CR, complete response; F, female; ns, not significant; NSG, NOD scid gamma or NOD.Cg-Prkdc^{scid} Il2rg^{tm1Wjl}/SzJ; q3d-q4d, twice a week; TGI, tumor growth inhibition.
p ≤ 0.05 versus control except where noted as ns.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of treating multiple myeloma in a subject in need thereof, comprising administering a therapeutically effective amount of a BCMA×GPRC5D×CD3 trispecific antibody or a trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of at least about 20 mg.

2. The method of claim 1, wherein the BCMA×GPRC5D×CD3 trispecific antibody or a trispecific binding fragment thereof and the anti-CD38 antibody treatment dose, respectively, is a flat dose.

3. The method of claim 1 or claim 2, wherein the BCMA×GPRC5D×CD3 trispecific antibody or a trispecific binding fragment thereof treatment dose is about 50 mg, about 100 mg, about 200 mg, or about 300 mg.

4. The method of claim 1, wherein the BCMA×GPRC5D×CD3 trispecific antibody or a trispecific binding fragment thereof treatment dose is about 100 mg, 200 mg, or 300 mg.

5. The method of claim 4, further comprising administering lenalidomide to the subject.

6. The method of claim 5, wherein lenalidomide is administered orally at a dose of about 25 mg daily for 21 days during a 28-day cycle, commencing on Cycle 2 Day 1.

7. The method of claim 5 or claim 6, wherein the dose of lenalidomide is adjusted based on patient tolerance and hematologic toxicity.

8. The method of any one of claims 5-7 further comprising administering dexamethasone in conjunction with lenalidomide.

9. The method of claim 8, wherein dexamethasone is administered at a dose of about 20 mg or 40 mg orally on Days 1, 8, 15, and 22 of the 28-day cycle.

10. The method of claim 9, wherein dexamethasone is administered for three 28-day cycles, commencing on Cycle 2 Day 1.

11. The method of any one of claims 1-10, wherein the anti-CD38 antibody is daratumumab.

12. The method of claim 11, wherein daratumumab is formulated in a pharmaceutical composition and administered subcutaneously.

13. The method of claim 11 or 12, wherein daratumumab is formulated in a pharmaceutical composition comprising 1800 mg daratumumab and 30000 units hyaluronidase.

14. The method of any one of claims 11-13, wherein daratumumab is formulated in a pharmaceutical composition comprising 1800 mg daratumumab and 30000 units hyaluronidase and is administered subcutaneously.

15. The method of any one of claims 1 to 14, wherein the BCMA×GPRC5D×CD3 trispecific antibody or a trispecific binding fragment thereof treatment dose is about 50 mg.

16. The method of any one of claims 1-14, wherein the BCMA×GPRC5D×CD3 trispecific antibody or a trispecific binding fragment thereof treatment dose is about 100 mg.

17. The method of any one of claims 1-14, wherein the BCMA×GPRC5D×CD3 trispecific antibody or a trispecific binding fragment thereof treatment dose is about 200 mg.

18. The method of any one of claims 1-14, wherein the BCMA×GPRC5D×CD3 trispecific antibody or a trispecific binding fragment thereof treatment dose is about 300 mg.

19. The method of any one of claims 1-18, wherein the treatment dose of the BCMA×GPRC5D×CD3 trispecific antibody or trispecific binding fragment thereof is administered once every four weeks.

20. The method of any one of claims 11-19, wherein the treatment dose of Daratumumab is administered once every four weeks.

21. The method of any one of claims 11-19, wherein the treatment dose of Daratumumab is administered once every week for two 28-day cycles, followed by administration once every two weeks for four 28-day cycles, followed by administration once every four weeks for subsequent 28-day cycles.

22. The method of any one of claims 1-21, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.

23. The method of claim 22, wherein the first step-up dose is about 5 mg.

24. The method of claim 22 or 23, wherein the first step-up dose is subcutaneously administered about 2-8 days prior to the subcutaneous administration of the treatment dose.

25. The method of claim 22 or 23, wherein the first step-up dose is subcutaneously administered about one week prior to the subcutaneous administration of the treatment dose.

26. The method of claim 22 or 23, wherein the first step-up dose is subcutaneously administered about 6-8 days prior to the subcutaneous administration of the treatment dose.

27. The method of claim 22 or 23, wherein the first step-up dose is subcutaneously administered about 2-4 days prior to the subcutaneous administration of the treatment dose.

28. The method of any one of claims 22 to 24, wherein the first step-up dose is subcutaneously administered about 2 to 8 days prior to the subcutaneous administration of the treatment dose, and wherein the first step-up dose is about 5 mg.

29. The method of any one of claim 22, 23 or 25, wherein the first step-up dose is subcutaneously administered about one week prior to the subcutaneous administration of the treatment dose, and wherein the first step-up dose is about 5 mg.

30. The method of any one of claim 22, 23 or 26, wherein the first step-up dose is subcutaneously administered about 6-8 days prior to the subcutaneous administration of the treatment dose, and wherein the first step-up dose is about 5 mg.

31. The method of any one of claim 22 or 27, wherein the first step-up dose is subcutaneously administered about 2-4 days prior to the subcutaneous administration of the treatment dose, and wherein the first step-up dose is about 2.5 mg.

32. The method of any one of claim 22, 23 or 27, wherein the first step-up dose is subcutaneously administered about 2-4 days prior to the subcutaneous administration of the treatment dose, and wherein the first step-up dose is about 5 mg.

33. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 50 mg and wherein the treatment dose is administered once every four weeks.

34. The method of claim 33, further comprising administering lenalidomide to the subject.

35. The method of claim 34, wherein lenalidomide is administered orally at a dose of about 25 mg daily for 21 days during a 28-day cycle, commencing on Cycle 2 Day 1.

36. The method of claim 34 or claim 35, wherein the dose of lenalidomide is adjusted based on patient tolerance and hematologic toxicity.

37. The method of any one of claims 34-36 further comprising administering dexamethasone in conjunction with lenalidomide.

38. The method of claim 37, wherein dexamethasone is administered at a dose of about 20 mg or 40 mg orally on Days 1, 8, 15, and 22 of the 28-day cycle.

39. The method of claim 38, wherein dexamethasone is administered for three 28-day cycles, commencing on Cycle 2 Day 1.

40. The method of any one of claims 33-39, wherein the anti-CD38 antibody is daratumumab.

41. The method of claim 40, wherein daratumumab is administered subcutaneously.

42. The method of claim 40 or 41, wherein daratumumab is formulated in a pharmaceutical composition comprising 1800 mg daratumumab and 30000 units hyaluronidase.

43. The method of any one of claims 40 to 42, wherein daratumumab is formulated in a pharmaceutical composition comprising 1800 mg daratumumab and 30000 units hyaluronidase and is administered subcutaneously.

44. The method of any one of claims 40 to 43, wherein daratumumab is administered once every four weeks.

45. The method of any one of claims 40 to 43, wherein daratumumab is administered once every week for two 28-day cycles, followed by administration once every two weeks for four 28-day cycles, followed by administration once every four weeks for subsequent 28-day cycles.

46. The method of any one of claims 33-45, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.

47. The method of claim 46, wherein the first step-up dose is about 2.5 mg and is administered about 2-4 days prior to administration of the treatment dose.

48. The method of claim 46, wherein the first step-up dose is about 5 mg and is administered about 2-4 days prior to administration of the treatment dose.

49. The method of claim 46, wherein the first step-up dose is about 5 mg and is administered about 2 to 8 days prior to administration of the treatment dose.

50. The method of claim 46, wherein the first step-up dose is about 5 mg and is administered about one week prior to administration of the treatment dose.

51. The method of claim 46, wherein the first step-up dose is about 5 mg and is administered about 6-8 days prior to administration of the treatment dose.

52. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 100 mg and wherein the treatment dose is administered once every four weeks.

53. The method of claim 52, further comprising administering lenalidomide to the subject.

54. The method of claim 53, wherein lenalidomide is administered orally at a dose of about 25 mg daily for 21 days during a 28-day cycle, commencing on Cycle 2 Day 1.

55. The method of claim 53 or claim 54, wherein the dose of lenalidomide is adjusted based on patient tolerance and hematologic toxicity.

56. The method of any one of claims 53-55 further comprising administering dexamethasone in conjunction with lenalidomide.

57. The method of claim 56, wherein dexamethasone is administered at a dose of about 20 mg or 40 mg orally on Days 1, 8, 15, and 22 of the 28-day cycle.

58. The method of claim 57, wherein dexamethasone is administered for three 28-day cycles, commencing on Cycle 2 Day 1.

59. The method of any one of claims 52-58, wherein the anti-CD38 antibody is daratumumab.

60. The method of claim 59, wherein daratumumab is administered subcutaneously.

61. The method of claim 59 or 60, wherein daratumumab is formulated in a pharmaceutical composition comprising 1800 mg daratumumab and 30000 units hyaluronidase.

62. The method of any one of claims 59 to 61, wherein daratumumab is formulated in a pharmaceutical composition comprising 1800 mg daratumumab and 30000 units hyaluronidase and is administered subcutaneously.

63. The method of any one of claims 59 to 62, wherein daratumumab is administered once every four weeks.

64. The method of any one of claims 59 to 62, wherein daratumumab is administered once every week for two 28-day cycles, followed by administration once every two weeks for four 28-day cycles, followed by administration once every four weeks for subsequent 28-day cycles.

65. The method of any one of claims 52-64, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.

66. The method of claim 65, wherein the first step-up dose is about 5 mg and is administered about 2-4 days prior to administration of the treatment dose.

67. The method of claim 65, wherein the first step-up dose is about 2.5 mg and is administered about 2-4 days prior to administration of the treatment dose.

68. The method of claim 65, wherein the first step-up dose is about 5 mg and is administered about 2-8 days prior to administration of the treatment dose.

69. The method of claim 65, wherein the first step-up dose is about 5 mg and is administered about one week prior to administration of the treatment dose.

70. The method of claim 65, wherein the first step-up dose is about 5 mg and is administered about 6-8 days prior to administration of the treatment dose.

71. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 200 mg and wherein the treatment dose is administered once every four weeks.

72. The method of claim 71, further comprising administering lenalidomide to the subject.

73. The method of claim 72, wherein lenalidomide is administered orally at a dose of about 25 mg daily for 21 days during a 28-day cycle, commencing on Cycle 2 Day 1.

74. The method of claim 72 or claim 73, wherein the dose of lenalidomide is adjusted based on patient tolerance and hematologic toxicity.

75. The method of any one of claims 72-74 further comprising administering dexamethasone in conjunction with lenalidomide.

76. The method of claim 75, wherein dexamethasone is administered at a dose of about 20 mg or 40 mg orally on Days 1, 8, 15, and 22 of the 28-day cycle.

77. The method of claim 76, wherein dexamethasone is administered for three 28-day cycles, commencing on Cycle 2 Day 1.

78. The method of any one of claims 71-77, wherein the anti-CD38 antibody is daratumumab.

79. The method of claim 78, wherein daratumumab is administered subcutaneously.

80. The method of claim 78 or 79, wherein daratumumab is formulated in a pharmaceutical composition comprising 1800 mg daratumumab and 30000 units hyaluronidase.

81. The method of any one of claims 78 to 80, wherein daratumumab is formulated in a pharmaceutical composition comprising 1800 mg daratumumab and 30000 units hyaluronidase and is administered subcutaneously.

82. The method of any one of claims 78 to 81, wherein daratumumab is administered once every four weeks.

83. The method of any one of claims 78 to 81, wherein daratumumab is administered once every week for two 28-day cycles, followed by administration once every two weeks for four 28-day cycles, followed by administration once every four weeks for subsequent 28-day cycles.

84. The method of any one of claims 71-83, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.

85. The method of claim 84, wherein the first step-up dose is about 5 mg and is administered about 2-4 days prior to administration of the treatment dose.

86. The method of claim 84, wherein the first step-up dose is about 2.5 mg and is administered about 2-4 days prior to administration of the treatment dose.

87. The method of claim 84, wherein the first step-up dose is about 5 mg and is administered about 2-8 days prior to administration of the treatment dose.

88. The method of claim 84, wherein the first step-up dose is about 5 mg and is administered about one week prior to administration of the treatment dose.

89. The method of claim 84, wherein the first step-up dose is about 5 mg and is administered about 6-8 days prior to administration of the treatment dose.

90. A method of treating multiple myeloma in a subject in need thereof, comprising administering a therapeutically effective amount of a BCMA×GPRC5D×CD3 trispecific antibody or trispecific binding fragment thereof and an anti-CD38 antibody, to the subject to treat the multiple myeloma, wherein the BCMA×GPRC5D×CD3 trispecific antibody or trispecific binding fragment thereof is subcutaneously administered at a treatment dose of about 300 mg and wherein the treatment dose is administered once every four weeks.

91. The method of claim 90, further comprising administering lenalidomide to the subject.

92. The method of claim 91, wherein lenalidomide is administered orally at a dose of about 25 mg daily for 21 days during a 28-day cycle, commencing on Cycle 2 Day 1.

93. The method of claim 91 or claim 92, wherein the dose of lenalidomide is adjusted based on patient tolerance and hematologic toxicity.

94. The method of any one of claims 91-93 further comprising administering dexamethasone in conjunction with lenalidomide.

95. The method of claim 94, wherein dexamethasone is administered at a dose of about 20 mg or 40 mg orally on Days 1, 8, 15, and 22 of the 28-day cycle.

96. The method of claim 95, wherein dexamethasone is administered for three 28-day cycles, commencing on Cycle 2 Day 1.

97. The method of any one of claims 90-96, wherein the anti-CD38 antibody is daratumumab.

98. The method of claim 97, wherein daratumumab is administered subcutaneously.

99. The method of claim 97 or 98, wherein daratumumab is formulated in a pharmaceutical composition comprising 1800 mg daratumumab and 30000 units hyaluronidase.

100. The method of any one of claims 97 to 99, wherein daratumumab is formulated in a pharmaceutical composition comprising 1800 mg daratumumab and 30000 units hyaluronidase and is administered subcutaneously.

101. The method of any one of claims 97 to 100, wherein daratumumab is administered once every four weeks.

102. The method of any one of claims 97 to 100, wherein daratumumab is administered once every week for two 28-day cycles, followed by administration once every two weeks for four 28-day cycles, followed by administration once every four weeks for subsequent 28-day cycles.

103. The method of any one of claims 90-102, wherein at least one step-up dose of the BCMA×GPRC5D×CD3 trispecific antibody or trispecific binding fragment thereof is subcutaneously administered prior to the subcutaneous administration of the treatment dose of the BCMA×GPRC5D×CD3 trispecific antibody or trispecific binding fragment thereof.

104. The method of claim 103, wherein the first step-up dose is about 5 mg and is administered about 2-4 days prior to administration of the treatment dose.

105. The method of claim 103, wherein the first step-up dose is about 2.5 mg and is administered about 2-4 days prior to administration of the treatment dose.

106. The method of claim 103, wherein the first step-up dose is about 5 mg and is administered about 2-8 days prior to administration of the treatment dose.

107. The method of claim 103, wherein the first step-up dose is about 5 mg and is administered about one week prior to administration of the treatment dose.

108. The method of claim 103, wherein the first step-up dose is about 5 mg and is administered about 6-8 days prior to administration of the treatment dose.

109. The method of any one of claims 1-108, wherein the BCMA×GPRC5D×CD3 trispecific antibody or trispecific binding fragment thereof comprises:
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2);
(c) a third antigen-binding arm comprising a third heavy chain variable domain (VH3) and a third light chain variable domain (VL3),
wherein the first antigen-binding arm binds to an epitope on cluster of differentiation 3 (CD3), the second antigen-binding arm binds to an epitope on G-protein coupled receptor family C group 5 member D (GPRC5D), and the third antigen-binding arm binds to an epitope on B cell maturation antigen (BCMA).

110. The method of claim 109, wherein the VH1 and VL1 of first antigen-binding arm are present in a Fab, the VH2 and VL2 of the second antigen-binding arm are present in a scFv, and the VH3 and VL3 of the third antigen-binding arm are present in a scFv.

111. The method of claim 109 or claim 110, wherein the first antigen-binding arm that binds CD3 comprises a HCDR1 comprising the amino acid sequence of GDSVENNNAAWS (SEQ ID NO: 4), a HCDR2 comprising the amino acid sequence of RTYYRSKWLYD (SEQ ID NO: 5), and a HCDR3 comprising the amino acid sequence of GYSSSFDY (SEQ ID NO: 6); and a LCDR1 comprising the amino acid sequence of TGTSSNIGTYKFVS (SEQ ID NO: 1), a LCDR2 comprising the amino acid sequence of EVSKRPS (SEQ ID NO: 2), and a LCDR3 comprising the amino acid sequence of VSYAGSGTLL (SEQ ID NO: 3).

112. The method of any one of claims 109-111, wherein the first antigen-binding arm that binds CD3 comprises the VH1 of SEQ ID NO: 8 and the VL1 of SEQ ID NO: 7.

113. The method of any one of claims 109-112, wherein the second antigen-binding arm that binds GPRC5D comprises a HCDR1 comprising the amino acid sequence of GFSLTNIRMSVS (SEQ ID NO: 12), HCDR2 comprising the amino acid sequence of HIFSNDEKS (SEQ ID NO: 13), and a HCDR3 comprising the amino acid sequence of MRLPYGMDV (SEQ ID NO: 14); and a LCDR1 comprising the amino acid sequence of RSSQSLVHSDGNTYLS (SEQ ID NO: 9), a LCDR2 comprising the amino acid sequence of KISNRFF (SEQ ID NO: 10), and a LCDR3 comprising the amino acid sequence of MQATQFPHT (SEQ ID NO: 11).

114. The method of any one of claims 109-113, wherein the second antigen-binding arm that binds GPRC5D comprises the VH2 of SEQ ID NO: 16 and the VL2 of SEQ ID NO: 15.

115. The method of any one of claims 109-114, wherein the third antigen-binding arm that binds BCMA comprises a HCDR1 comprising the amino acid sequence of GFTFSSYAMS (SEQ ID NO: 20), a HCDR2 comprising the amino acid sequence of AISGSGGSTY (SEQ ID NO: 21), and a HCDR3 comprising the amino acid sequence of DEGYSSGHYYGMDV (SEQ ID NO: 22); and a LCDR1 comprising the amino acid sequence of RASQSISSSFLT (SEQ ID NO: 17), a LCDR2 comprising the amino acid sequence of GASSRAT (SEQ ID NO: 18), and a LCDR3 comprising the amino acid sequence of QHYGSSPMYT (SEQ ID NO: 19).

116. The method of any one of claims 109-115, wherein the third antigen-binding arm that binds BCMA comprises the VH3 of SEQ ID NO: 24 and the VL3 of SEQ ID NO: 23.

117. The method of any one of claims 109-116, wherein the first antigen-binding arm that binds CD3 comprises the HCDR1, the HCDR2, the HCDR3, the LCDR1, the LCDR2 and the LCDR3 of SEQ ID NOs: 4, 5, 6, 1, 2, 3, respectively; the second antigen-binding arm that binds GPRC5D comprises the HCDR1, the HCDR2, the HCDR3, the LCDR1, the LCDR2 and the LCDR3 of SEQ ID NOS: 12, 13, 14, 9, 10 and 11, respectively; and the third antigen-binding arm that binds BCMA comprises the HCDR1, the HCDR2, the HCDR3, the LCDR1, the LCDR2 and the LCDR3 of SEQ ID NOs: 20, 21, 22, 17, 18 and 19, respectively.

118. The method of any one of claims 109-117, wherein the first antigen-binding arm that binds CD3 comprises the VH1 of SEQ ID NO: 8 and the VL1 of SEQ ID NO: 7; the second antigen-binding arm that binds GPRC5D comprises the VH2 of SEQ ID NO: 16 and the VL2 of SEQ ID NO: 15; and the third antigen-binding arm that binds BCMA comprises the VH3 of SEQ ID NO: 24 and the VL3 of SEQ ID NO: 23.

119. The method of any one of claims 109-118, wherein the BCMA×GPRC5D×CD3 trispecific antibody comprises a first antigen-binding arm that binds to an epitope on cluster of differentiation 3 (CD3), a second antigen-binding arm that binds to an epitope on G-protein coupled receptor family C group 5 member D (GPRC5D), and a third antigen-binding arm that binds to an epitope on B cell maturation antigen (BCMA), wherein the first antigen-binding arm comprises a heavy chain (HC1) polypeptide and a light chain (LC) polypeptide, wherein the heavy chain (HC1) polypeptide further comprises the second antigen-binding arm, wherein the trispecific antibody, or the trispecific binding fragment thereof, further comprises a single polypeptide comprising the third antigen-binding arm.

120. The method of claim 119, wherein the HC1 of the first antigen-binding arm comprises the amino acid sequence of SEQ ID NO: 29.

121. The method of claim 119 or claim 120, wherein the LC of the first antigen-binding arm comprises the amino acid sequence of SEQ ID NO: 30.

122. The method of any one of claims 119-121, wherein the single polypeptide comprising the third antigen-binding arm comprises an amino acid sequence of SEQ ID NO: 31.

123. The method of any one of claims 119-122, wherein the first antigen-binding arm comprises an HC1 comprising the amino acid sequence of SEQ ID NO: 29, and a LC comprising the amino acid sequence of SEQ ID NO: 30, and the single polypeptide comprising the third antigen-binding arm comprises the amino acid sequence of SEQ ID NO: 31.

124. The method of any one of claims 109-123, wherein the BCMA×GPRC5D×CD3 trispecific antibody or trispecific binding fragment thereof is an (human) IgG1 isotype.

125. The method of any one of claims 1-124, wherein the multiple myeloma is newly diagnosed multiple myeloma.

126. The method of claim 125, wherein the subject exhibits high-risk cytogenetic abnormalities selected from the group consisting of del(17p), t(4;14), t(14;16), and 1q21 gain/1q21 amplification.

127. The method of claim 126, wherein the high-risk cytogenetic abnormalities are identified using fluorescence in situ hybridization (FISH) or next-generation sequencing (NGS).

128. The method of any one of claims 1-124, wherein the multiple myeloma is relapsed or refractory multiple myeloma (RRMM).

129. The method of claim 128, wherein the relapsed or refractory multiple myeloma is relapsed multiple myeloma.

130. The method of claim 128, wherein the relapsed or refractory multiple myeloma is refractory multiple myeloma.

131. The method of any one of claims 1-124 and 128-130 wherein the subject is relapsed or refractory to treatment with a prior anti-cancer treatment.

132. The method of claim 131, wherein the prior anti-cancer treatment comprises administering at least one of a proteasome inhibitor and immunomodulatory drug, such as bortezomib, carfilzomib, lenalidomide, or pomalidomide.

133. The method of any one of claims 1-124 and 128-132 wherein the subject has received a prior treatment.

134. The method of claim 133, wherein the prior treatment comprises a proteasome inhibitor, and/or an immunomodulatory drug, or any combination thereof.

135. The method of any one of claims 1-124 and 128-134, wherein the subject has received >1 lines of prior treatment.

136. The method of any one of claims 1-124 and 128-134, wherein the subject has received 1-3 lines of prior treatment.

137. The method of claim 135 or claim 136, wherein the subject is relapsed or refractory to the lines of prior treatment.

138. The method of any one of claims 1-137, wherein the subject is a human subject.

139. A pharmaceutical composition comprising a BCMA×GPRC5D×CD3 trispecific antibody or a trispecific binding fragment thereof for use in treating multiple myeloma, wherein the composition is for administration, or to be administered, in combination with a pharmaceutical composition comprising an anti-CD38 antibody, and further wherein the BCMA×GPRC5D×CD3 trispecific antibody or trispecific binding fragment thereof is for subcutaneous administration at a treatment dose of at least about 20 mg.

140. A pharmaceutical composition comprising an anti-CD38 antibody for use in treating multiple myeloma, wherein the composition is for administration, or to be administered, in combination with a pharmaceutical composition comprising a BCMA×GPRC5D×CD3 trispecific antibody or a trispecific binding fragment thereof, and further wherein the BCMA×GPRC5D×CD3 trispecific antibody or trispecific binding fragment thereof is for subcutaneous administration at a treatment dose of at least about 20 mg.

141. A pharmaceutical composition for use, as claimed in claim 139 or claim 140, wherein the anti-CD38 antibody comprises a heavy chain complementarity determining region 1 (HCDR1) of SEQ ID NO: 201, a HCDR2 of SEQ ID NO: 202, a HCDR3 of SEQ ID NO: 203, a light chain complementarity determining region 1 (LCDR1) of SEQ ID NO: 204, a LCDR2 of SEQ ID NO: 205 and a LCDR3 of SEQ ID NO: 206.

142. A pharmaceutical composition for use, as claimed in claim 141, wherein the anti-CD-38 antibody comprises a heavy chain variable region (VH) of SEQ ID NO: 207 and a light chain variable region (VL) of SEQ ID NO: 208.

143. A pharmaceutical composition for use, as claimed in claim 141 or claim 142, wherein the anti-CD38 antibody is an IgG1 isotype.

144. A pharmaceutical composition for use, as claimed in any one of claims 139 to 143, wherein the anti-CD38 antibody comprises a heavy chain (HC) of SEQ ID NO: 209 and a light chain (LC) of SEQ ID NO: 210.

145. A pharmaceutical composition for use, as claimed in any one of claims 139 to 144, wherein the anti-CD-38 antibody is daratumumab.

146. A pharmaceutical composition for use, as claimed in any one of claims 139-145, wherein the anti-CD38 antibody is formulated in a pharmaceutical formulation further comprising a hyaluronidase, optionally, wherein the hyaluronidase is a recombinant human hyaluronidase, optionally wherein the recombinant human hyaluronidase is rHuPH20.

147. A pharmaceutical composition for use as claimed in any one of claims 139-146, wherein the pharmaceutical composition comprising an anti-CD38 antibody is administered subcutaneously.

148. A pharmaceutical composition for use as claimed in any one of claims 139-147, wherein the pharmaceutical composition comprising an anti-CD38 antibody is formulated in a pharmaceutical composition comprising 1800 mg daratumumab and 30000 units hyaluronidase.

149. A pharmaceutical composition for use as claimed in any one of claims 139 to 148, wherein the BCMA×GPRC5D×CD3 trispecific antibody or trispecific binding fragment thereof comprises:
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2);
(c) a third antigen-binding arm comprising a third heavy chain variable domain (VH3) and a third light chain variable domain (VL3),
wherein the first antigen-binding arm binds to an epitope on cluster of differentiation 3 (CD3), the second antigen-binding arm binds to an epitope on G-protein coupled receptor family C group 5 member D (GPRC5D), and the third antigen-binding arm binds to an epitope on B cell maturation antigen (BCMA).

150. The pharmaceutical composition for use as claimed in claim 149, wherein the VH1 and VL1 of first antigen-binding arm are present in a Fab, the VH2 and VL2 of the second antigen-binding arm are present in a scFv, and the VH3 and VL3 of the third antigen-binding arm are present in a scFv.

151. The pharmaceutical composition for use as claimed in claim 149 or 150, wherein the first antigen-binding arm that binds CD3 comprises a HCDR1 comprising the amino acid sequence of GDSVENNNAAWS (SEQ ID NO: 4), a HCDR2 comprising the amino acid sequence of RTYYRSKWLYD (SEQ ID NO: 5), and a HCDR3 comprising the amino acid sequence of GYSSSFDY (SEQ ID NO: 6); and a LCDR1 comprising the amino acid sequence of TGTSSNIGTYKFVS (SEQ ID NO: 1), a LCDR2 comprising the amino acid sequence of EVSKRPS (SEQ ID NO: 2), and a LCDR3 comprising the amino acid sequence of VSYAGSGTLL (SEQ ID NO: 3).

152. The pharmaceutical composition for use as claimed in any one of claims 149 to 151, wherein the first antigen-binding arm that binds CD3 comprises the VH1 of SEQ ID NO: 8 and the VL1 of SEQ ID NO: 7.

153. The pharmaceutical composition for use as claimed in any one of claims 149 to 152, wherein the second antigen-binding arm that binds GPRC5D comprises a HCDR1 comprising the amino acid sequence of GFSLTNIRMSVS (SEQ ID NO: 12), HCDR2 comprising the amino acid sequence of HIFSNDEKS (SEQ ID NO: 13), and a HCDR3 comprising the amino acid sequence of MRLPYGMDV (SEQ ID NO: 14); and a LCDR1 comprising the amino acid sequence of RSSQSLVHSDGNTYLS (SEQ ID NO: 9), a LCDR2 comprising the amino acid sequence of KISNRFF (SEQ ID NO: 10), and a LCDR3 comprising the amino acid sequence of MQATQFPHT (SEQ ID NO: 11).

154. The pharmaceutical composition for use as claimed in any one of claims 149 to 153, wherein the second antigen-binding arm that binds GPRC5D comprises the VH2 of SEQ ID NO: 16 and the VL2 of SEQ ID NO: 15.

155. The pharmaceutical composition for use as claimed in any one of claims 149 to 154, wherein the third antigen-binding arm that binds BCMA comprises a HCDR1 comprising the amino acid sequence of GFTFSSYAMS (SEQ ID NO: 20), a HCDR2 comprising the amino acid sequence of AISGSGGSTY (SEQ ID NO: 21), and a HCDR3 comprising the amino acid sequence of DEGYSSGHYYGMDV (SEQ ID NO: 22); and a LCDR1 comprising the amino acid sequence of RASQSISSSFLT (SEQ ID NO: 17), a LCDR2 comprising the amino acid sequence of GASSRAT (SEQ ID NO: 18), and a LCDR3 comprising the amino acid sequence of QHYGSSPMYT (SEQ ID NO: 19).

156. The pharmaceutical composition for use as claimed in any one of claims 149 to 155, wherein the third antigen-binding arm that binds BCMA comprises the VH3 of SEQ ID NO: 24 and the VL3 of SEQ ID NO: 23.

157. The pharmaceutical composition for use as claimed in any one of claims 149 to 156, wherein the first antigen-binding arm that binds CD3 comprises the HCDR1, the HCDR2, the HCDR3, the LCDR1, the LCDR2 and the LCDR3 of SEQ ID NOs: 4, 5, 6, 1, 2, 3, respectively; the second antigen-binding arm that binds GPRC5D comprises the HCDR1, the HCDR2, the HCDR3, the LCDR1, the LCDR2 and the LCDR3 of SEQ ID NOs: 12, 13, 14, 9, 10 and 11, respectively; and the third antigen-binding arm that binds BCMA comprises the HCDR1, the HCDR2, the HCDR3, the LCDR1, the LCDR2 and the LCDR3 of SEQ ID NOs: 20, 21, 22, 17, 18 and 19, respectively.

158. The pharmaceutical composition for use as claimed in any one of claims 149 to 157, wherein the first antigen-binding arm that binds CD3 comprises the VH1 of SEQ ID NO: 8 and the VL1 of SEQ ID NO: 7; the second antigen-binding arm that binds GPRC5D comprises the VH2 of SEQ ID NO: 16 and the VL2 of SEQ ID NO: 15; and the third antigen-binding arm that binds BCMA comprises the VH3 of SEQ ID NO: 24 and the VL3 of SEQ ID NO: 23.

159. The pharmaceutical composition for use as claimed in any one of claims 149 to 158, wherein the BCMA×GPRC5D×CD3 trispecific antibody comprises a first antigen-binding arm that binds to an epitope on cluster of differentiation 3 (CD3), a second antigen-binding arm that binds to an epitope on G-protein coupled receptor family C group 5 member D (GPRC5D), and a third antigen-binding arm that binds to an epitope on B cell maturation antigen (BCMA), wherein the first antigen-binding arm comprises a heavy chain (HC1) polypeptide and a light chain (LC) polypeptide, wherein the heavy chain (HC1) polypeptide further comprises the second antigen-binding arm, wherein the trispecific antibody, or the trispecific binding fragment thereof, further comprises a single polypeptide comprising the third antigen-binding arm.

160. The pharmaceutical composition for use as claimed in claim 159, wherein the HC1 of the first antigen-binding arm comprises the amino acid sequence of SEQ ID NO: 29.

161. The pharmaceutical composition for use as claimed in claim 159 or 160, wherein the LC of the first antigen-binding arm comprises the amino acid sequence of SEQ ID NO: 30.

162. The pharmaceutical composition for use as claimed in any one of claims 159 to 161, wherein the single polypeptide comprising the third antigen-binding arm comprises an amino acid sequence of SEQ ID NO: 31.

163. The pharmaceutical composition for use as claimed in any one of claims 159 to 162, wherein the first antigen-binding arm comprises an HC1 comprising the amino acid sequence of SEQ ID NO: 29, and a LC comprising the amino acid sequence of SEQ ID NO: 30, and the single polypeptide comprising the third antigen-binding arm comprises the amino acid sequence of SEQ ID NO: 31.

164. The pharmaceutical composition for use as claimed in any one of claims 159 to 163, wherein the BCMA×GPRC5D×CD3 trispecific antibody or trispecific binding fragment thereof is an (human) IgG1 isotype.

165. The pharmaceutical composition for use as claimed in any one of claims 159 to 164, wherein the BCMA×GPRC5D×CD3 trispecific antibody or trispecific binding fragment thereof is for subcutaneous administration at a treatment dose of at least about 50 mg.

166. The pharmaceutical composition for use as claimed in any one of claims 159 to 164, wherein the BCMA×GPRC5D×CD3 trispecific antibody or trispecific binding fragment thereof is for subcutaneous administration at a treatment dose of at least about 100 mg.

167. The pharmaceutical composition for use as claimed in any one of claims 159 to 164, wherein the BCMA×GPRC5D×CD3 trispecific antibody or trispecific binding fragment thereof is for subcutaneous administration at a treatment dose of at least about 200 mg.

168. The pharmaceutical composition for use as claimed in any one of claims 159 to 164, wherein the BCMA×GPRC5D×CD3 trispecific antibody or trispecific binding fragment thereof is for subcutaneous administration at a treatment dose of at least about 300 mg.

169. The pharmaceutical composition for use as claimed in claims 139 to 168, wherein the pharmaceutical composition further comprises lenalidomide for oral administration at a dosage of 25 mg per day.

170. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg; and
(d) the subject has received 1-3 lines of prior treatment.

171. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
(d) the multiple myeloma is newly diagnosed multiple myeloma; and
(e) the subject exhibits high-risk cytogenetic abnormalities selected from the group consisting of del(17p), t(4;14), t(14;16), and 1q21 gain/1q21 amplification.

172. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
(d) the multiple myeloma is newly diagnosed multiple myeloma; and
(e) the subject exhibits high-risk cytogenetic abnormalities selected from the group consisting of del(17p), t(4;14), t(14;16), and 1q21 gain/1q21 amplification.

173. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
(d) the method further comprises administering lenalidomide to the subject, wherein lenalidomide is administered orally at a dose of about 25 mg daily for 21 days during a 28-day cycle, commencing on Cycle 2 Day 1;
(e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
(f) the multiple myeloma is newly diagnosed multiple myeloma.

174. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg;
(d) the method further comprises administering lenalidomide to the subject, wherein lenalidomide is administered orally at a dose of about 25 mg daily for 21 days during a 28-day cycle, commencing on Cycle 2 Day 1;
(e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
(f) the multiple myeloma is newly diagnosed multiple myeloma.

175. The method of any one of claims 170-174, wherein the BCMA×GPRC5D×CD3 trispecific antibody treatment dose is about 50 mg, about 100 mg, about 200 mg, or about 300 mg.

176. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg; and
(d) the subject has received 1-3 lines of prior treatment.

177. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
(d) the multiple myeloma is newly diagnosed multiple myeloma; and
(e) the subject exhibits high-risk cytogenetic abnormalities selected from the group consisting of del(17p), t(4;14), t(14;16), and 1q21 gain/1q21 amplification.

178. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
(d) the multiple myeloma is newly diagnosed multiple myeloma; and
(e) the subject exhibits high-risk cytogenetic abnormalities selected from the group consisting of del(17p), t(4;14), t(14;16), and 1q21 gain/1q21 amplification.

179. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered subcutaneously once every four weeks;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 and the treatment dose is about 50 mg, about 100 mg, about 200 mg, or about 300 mg;
(d) the method further comprises administering lenalidomide to the subject, wherein lenalidomide is administered orally at a dose of about 25 mg daily for 21 days during a 28-day cycle, commencing on Cycle 2 Day 1;
(e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
(f) the multiple myeloma is newly diagnosed multiple myeloma.

180. A method of treating multiple myeloma in a subject in need thereof, comprising administering a therapeutically effective amount of a BCMA×GPRC5D×CD3 trispecific antibody and an anti-CD38 antibody, to the subject to treat the multiple myeloma, wherein:
(a) the BCMA×GPRC5D×CD3 trispecific antibody is a BCMA×GPRC5D×CD3 trispecific antibody comprising:
(i) a HC1 comprising the amino acid sequence of SEQ ID NO: 29;
(ii) a LC comprising the amino acid sequence of SEQ ID NO: 30, and
(iii) a HC2 comprising an amino acid sequence of SEQ ID NO: 31,
and wherein the BCMA×GPRC5D×CD3 trispecific antibody is administered once every four weeks;
(b) the anti-CD38 antibody is daratumumab, wherein daratumumab is formulated in a pharmaceutical composition comprising 1800 mg daratumumab and 30000 units hyaluronidase and is administered once every week for two 28-day cycles, followed by administration once every two weeks for four 28-day cycles, followed by administration once every four weeks for subsequent 28-day cycles;
(c) at least one step-up dose of the BCMA×GPRC5D×CD3 trispecific antibody is subcutaneously administered prior to the subcutaneous administration of the treatment dose of the BCMA×GPRC5D×CD3 trispecific antibody, wherein the first step-up dose is subcutaneously administered about 2 to 8 days prior to the subcutaneous administration of the treatment dose, and wherein the first step-up dose is about 5 mg and the treatment dose is about 50 mg, about 100 mg, about 200 mg, or about 300 mg;
(d) the method further comprises administering lenalidomide to the subject, wherein lenalidomide is administered orally at a dose of about 25 mg daily for 21 days during a 28-day cycle, commencing on Cycle 2 Day 1;
(e) the method further comprises administering dexamethasone in conjunction with lenalidomide, wherein dexamethasone is administered at a dose of about 40 mg orally on Days 1, 8, 15, and 22 of the 28-day cycle, wherein dexamethasone is administered for three 28-day cycles, commencing on Cycle 2 Day 1; and
(f) the multiple myeloma is newly diagnosed multiple myeloma.


SEQUENCE LISTING (216 sequences)

SEQ ID NO: 1 - protein, 14 aa, synthetic construct
TGTSSNIGTYKFVS

SEQ ID NO: 2 - protein, 7 aa, synthetic construct
EVSKRPS

SEQ ID NO: 3 - protein, 10 aa, synthetic construct
VSYAGSGTLL

SEQ ID NO: 4 - protein, 12 aa, synthetic construct
GDSVFNNNAAWS

SEQ ID NO: 5 - protein, 11 aa, synthetic construct
RTYYRSKWLYD

SEQ ID NO: 6 - protein, 8 aa, synthetic construct
GYSSSFDY

SEQ ID NO: 7 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 8 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 9 - protein, 16 aa, synthetic construct
RSSQSLVHSDGNTYLS

SEQ ID NO: 10 - protein, 7 aa, synthetic construct
KISNRFF

SEQ ID NO: 11 - protein, 9 aa, synthetic construct
MQATQFPHT

SEQ ID NO: 12 - protein, 12 aa, synthetic construct
GFSLTNIRMSVS

SEQ ID NO: 13 - protein, 9 aa, synthetic construct
HIFSNDEKS

SEQ ID NO: 14 - protein, 9 aa, synthetic construct
MRLPYGMDV

SEQ ID NO: 15 - protein, 112 aa, synthetic construct
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIK

SEQ ID NO: 16 - protein, 119 aa, synthetic construct
QVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 17 - protein, 12 aa, synthetic construct
RASQSISSSFLT

SEQ ID NO: 18 - protein, 7 aa, synthetic construct
GASSRAT

SEQ ID NO: 19 - protein, 10 aa, synthetic construct
QHYGSSPMYT

SEQ ID NO: 20 - protein, 10 aa, synthetic construct
GFTFSSYAMS

SEQ ID NO: 21 - protein, 10 aa, synthetic construct
AISGSGGSTY

SEQ ID NO: 22 - protein, 14 aa, synthetic construct
DEGYSSGHYYGMDV

SEQ ID NO: 23 - protein, 109 aa, synthetic construct
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIK

SEQ ID NO: 24 - protein, 123 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 25 - protein, 20 aa, synthetic construct
GGSEGKSSGSGSESKSTGGS

SEQ ID NO: 26-28 - skipped (3 entries)

SEQ ID NO: 29 - protein, 721 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSGGGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 30 - protein, 216 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 484 aa, synthetic construct
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32-115 - skipped (84 entries)

SEQ ID NO: 116 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 117-157 - skipped (41 entries)

SEQ ID NO: 158 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - skipped

SEQ ID NO: 161 - protein, 14 aa, synthetic construct
QDGNEEMGGITQTP

SEQ ID NO: 162 - protein, 10 aa, synthetic construct
LLHACIPCQL

SEQ ID NO: 163 - protein, 20 aa, synthetic construct
GGGGSGGGGSGGGGSGGGGS

SEQ ID NO: 164 - skipped

SEQ ID NO: 165 - DNA, 2229 bp, synthetic construct
  source 1..2229: mol_type = other DNA
atggccaggaagtccgctctgctcgctctggcacttctgcttctgggatttggacctgcttgggctcaggtgcagctgcagcagtctggccctagactcgtgcggccttcccagaccctgtctctgacctgtgccatctccggcgactccgtgttcaacaacaacgccgcctggtcctggatccggcagtctccatctcgcggtctggagtggctcggtcgcacctactaccgctctaaatggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccaaggcaccctcgtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaaggaggcggaggatctggcggaggtggaagtggcggaggcggttctggtggtggtggatctgacatcgtgatgacccagacacctctgagcagccccgttacattgggccagcctgcctccatctcctgcagatcctctcagtccctggtgcactctgacggcaacacctacctctcttggctgcagcagaggcctggacagcctcctagactgctgatctacaagatctccaaccggttcttcggcgtgcccgacagattttctggatctggcgctggcaccgacttcaccctgaagatttccagagtggaagccgaggacgtgggcgtgtactactgtatgcaggccacacagttccctcacacctttggccagggcaccaagctggaaatcaagggcggatctgagggaaagtccagcggctccggcagcgaaagcaagtccaccggcggaagccaagtgaccctgaaagaaagcggccctgtgctggtcaagcccaccgaaacactgaccctgacctgtaccgtgtccggcttctccctgaccaatatccggatgtccgtgtcctggatcagacagcctcctggcaaggctctggaatggctggcccacatcttctccaacgacgagaagtcctactccaccagcctgaagtcccggctgaccatctccagagacacctccaagtctcaggtggtgctgacactgaccaacgtggaccctgtggataccgccacctactactgcgccagaatgagactgccctacggcatggatgtgtggggccagggaacaaccgtgaccgtttcttct

SEQ ID NO: 166 - DNA, 717 bp, synthetic construct
  source 1..717: mol_type = other DNA
atggctagatccgcactgctcattctggctctgcttctgcttggactgttctctcctggagcatggggacagtctgctctgacccagcctgcctccgtgtctggctctcccggccagtccatcaccatcagctgtaccggcacctcctccaacatcggcacctacaagttcgtgtcctggtatcagcaacaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgactgtcctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtcgaaaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 167 - DNA, 1518 bp, synthetic construct
  source 1..1518: mol_type = other DNA
atggccaggaagtccgctctgctcgctctggcacttctgcttctgggatttggacctgcttgggctgagatcgtgctgacccagtctcctggcacactgtcactgtctccaggcgagagagctaccctgtcctgtagagccagccagtctatctcctcctccttcctgacctggtatcagcagaagcctggacaggcccctcggctgttgatctacggtgcttcttccagagccacaggcatccctgacagattctctggcggcggatctggcaccgacttcaccctgacaatctcccggctggaacctgaggacttcgccgtgtactactgccagcactacggcagcagccccatgtacacatttggccagggcaccaagctggaaatcaagggcggatctgagggaaagtccagcggctccggcagcgaaagcaagtccaccggcggaagcgaggttcagctgctggaatctggcggaggattggttcagcctggcggttctctgagactgtcttgtgccgcttccggcttcaccttctccagctacgctatgtcctgggtccgacaggctcctggcaaaggactggaatgggtgtccgccatctctggctctggcggcagcacctactacgccgattctgtgaagggcagattcaccatcagccgggacaactccaagaacaccctgtacctgcagatgaactccctgagagccgaggacaccgccgtgtactactgtgctaaggacgagggctactcctccggccactactacggaatggatgtgtggggccagggcaccacagtgacagtctcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 168-200 - skipped (33 entries)

SEQ ID NO: 201 - protein, 5 aa, synthetic construct
SFAMS

SEQ ID NO: 202 - protein, 17 aa, synthetic construct
AISGSGGGTYYADSVKG

SEQ ID NO: 203 - protein, 13 aa, synthetic construct
DKILWFGEPVFDY

SEQ ID NO: 204 - protein, 11 aa, synthetic construct
RASQSVSSYLA

SEQ ID NO: 205 - protein, 7 aa, synthetic construct
DASNRAT

SEQ ID NO: 206 - protein, 9 aa, synthetic construct
QQRSNWPPT

SEQ ID NO: 207 - protein, 122 aa, synthetic construct [sequence omitted]

SEQ ID NO: 208 - protein, 107 aa, synthetic construct [sequence omitted]

SEQ ID NO: 209 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 443 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 213 - protein, 447 aa, synthetic construct [sequence omitted]

SEQ ID NO: 214 - protein, 446 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 215 - protein, 445 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 216 - protein, 444 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ